(19) United States
(12) Patent Application Publication
(10) Pub. No.: US 2023/0131943 A1
(43) Pub. Date: Apr. 27, 2023

(54) ANTI-INFECTIVE AND ANTI-VIRAL COMPOUNDS AND COMPOSITIONS

(71) Applicant: Micheal W. Burnet, Tuebingen (DE)

(72) Inventor: Michael W. Burnet, Tuebingen (DE)

(21) Appl. No.: 17/914,301

(22) PCT Filed: Mar. 23, 2021
(86) PCT No.: PCT/US2021/023749

§ 371 Date: Sep. 23, 2022

Related U.S. Application Data
(60) Provisional application No. 62/994,182, filed on Mar. 24, 2020.

(51) Int. Cl. A61K 31/7052; A61K 31/4706; A61P 31/04; A61K 31/222; A61K 31/138; A61K 31/4439; A61K 31/415; A61K 31/7135; A61K 33/30; A61K 31/133; A61K 31/5377; A61K 31/24; A61P 31/14

(52) U.S. Cl. CPC A61K 31/7052; A61K 31/4706; A61P 31/04; A61K 31/222; A61K 31/138; A61K 31/4439; A61K 31/415; A61K 31/7135; A61K 33/30; A61K 31/133; A61K 31/5377; A61K 31/24; A61P 31/14

(57) ABSTRACT

Lysosomally accumulated substances that release a nitroxy group, or a short chain fatty acid or a product of anaerobic metabolism or a thiol or a sulfide often from an ester or similar labile linkage have anti-inflammatory, anti-cancer and anti-bacterial activity. They are useful in treating infectious, inflammatory and malignant disease and are immune stimulatory, promote zinc uptake, disable endosomal reactions and synergise anti-viral action of protease inhibitors. The compounds are useful for the treatment of bacterial, viral and mixed pneumonias.

### BACKGROUND

The human body senses bacteria, fungi or other parasites by both recognition of their surface patterns and via reception of the metabolic products of those organisms. In particular, the immune system is sensitive to the presence of anaerobic organisms which, by their nature are able to infect poorly perfused tissue, or emanate from the anaerobic lumen of the gut. Anaerobes seem to be a particular source of danger signals for the immune system because they often produce toxins and are able to proliferate in the absence of oxygen, where immune cells are less active and less able to use oxidative burst to kill the bacteria they ingest.

Certain virus types are adapted to the gut and thus to low oxygen tensions. Corona virus SARS-CoV2 is one such example in that it is a gut commensal of some wild species, induces glycolysis, and makes intensive use of cysteine, an amino acid sensitive to oxidation. The induction of glycolysis is a form of anaerobic signal in infected cells and may be one reason that hyper immune responses are associated with some forms of the infection.

Amongst the signals that human cells respond to are the products of fermentation such as the short chain fatty acids (SCFAs) acetate, propionate and butyrate. Less characterised mediators with similar function include: lactic acid, H₂S, HS—, nitrite, polyamines and similar decarboxylated amino acids such as 3-Indolepropionic acid (IPA), deoxybile acids and polyphenol metabolites like phenylpropionic acid. In a most general sense, these metabolites are variously sensed by a range of receptors that include SCFA receptors (e.g. FFA2), the pregnane X receptor or the arylhydrocarbon receptor. For convenience, we will describe here these materials as Products of Anaerobic Metabolism (PAM or PAMS for the plural). Another type of signal is that of bacterial cell wall materials or bacterial nucleic acids. These materials are often ligands for the Toll-like-receptor (TLR) family. These are referred to as Pathogen-associated molecular patterns or PAMPs. For simplicity here, we will refer to all signal types as PAM or PAM s in the plural.

Although signaling of this type is commonly associated with the gut epithelium, it is also a potential signal in the interaction between the immune system surveilling the gut or the periphery. In particular, the lysosomes of the gut or the immune system cells are exposed to bacterial metabolites because these materials are released as bacteria are lysed following phagocytosis.

It is observed that delivering donors of SCFAs, TLR ligands, and other bacterial metabolites to the phagosome of immune cells results in immune stimulation such that phagocytosed bacteria are more rapidly killed. This effect can be augmented by the presentation of additional signals in parallel, for example nitric oxide (NO), delivered from a nitro ester.

Nitric oxide functions as a neurotransmitter, autacoid constitutive mediator, inducible mediator, cytoprotective molecule, and cytotoxic molecule. Since NO plays multiple physiological roles in the regulation of numerous and diverse organ functions, defects in the NO pathway lead to a variety of pathophysiological states. Possible disorders are: arteriosclerosis, hypertension, coronary artery disease, cardiac failure, pulmonary hypertension, stroke, impotence, gastrointestinal ulcers, asthma, and other CNS and systemic disorders^{[1]}.

Synthetic chemical reagents that release NO continuously over a period of time, under physiological conditions, have been in use for a long time in treatment of cardiovascular diseases^{[2]}. Most widely used are organic nitrates (e. g. glyceryl trinitrate): These NO donors need thiols as a cofactor for generating NO. They can use endogenous sources of thiols.

Other important series of NO donors are the so-called NOC, NOR, and NONOate compounds which were reviewed by Wang et al^{[3]}.

Furthermore a class of activators of soluble guanylyl cyclase (e. g. YC-1) is known as NO-sensitizer, which may potentiate the effect of minimal NO concentrations^{[4]}.

One major drawback of all those NO donors consists in the fact that they exert their action largely in the extracellular environment. For example, nitroglycerine leads to the release of NO in the plasma which stimulates vasodilation by its action on smooth muscle surrounding vessels.

A similar drawback for the use of short chain fatty acids or hydrogen sulfides as pharmaceutical agents is that they are potent odorants and required in relatively large amounts (the SCFA receptors have affinities in the milli-molar range). Thus they require specific delivery if they are to be effective.

Another difficulty in the use of NO donors is the synthetic methods. The preparation of nitro and nitrooxy compounds belongs to the most widespread examples for electrophilic substitution reactions. In general all methods for nitration lead to the formation of nitronium cations as electrophiles, in most cases generated in situ^{[5]}. Few examples are published employing NO species as a salt, e.g. nitronium tetrafluoroborate that can be employed for highly regioselective aromatic nitration^{[6]}. Obstacles common to all methods are, besides desired selectivity, relatively harsh reaction conditions: acidity, oxidizing reactants, and temperature. Thus, classical procedures are limited to stable systems that withstand these reaction conditions. Unfortunately this excludes many substance classes like drugs, organic molecules, reducing sugars or other natural products. Transformation of these compounds to corresponding nitrates would result in valuable compounds. Especially the case of O-nitration synthesis faces several problems: Starting materials or intermediates in many cases do not withstand conventional reaction conditions like HNO₃—H₂SO₄-mixtures. Thus the strategy of synthesis has to be changed, utilizing mild nitrating agents like acetyl nitrate^{[7,8,9]} or benzoyl nitrate^{[9,10,11,12]}, herein referred to as acyl nitrates. Chemically theses nitrating agents are mixed anhydrides from nitric acid and corresponding carboxylic acids, mainly generated in situ by reaction of a carboxylic anhydride or halides with nitric acid.

NO has many biological functions and as such can serve as a molecular warhead if appropriately delivered by a carrier molecule. In this regard it shares properties in common with compounds like CO and H₂S. Another class of small effect molecule of natural origin are the short chain fatty acids (SCFAs) alluded to above. These compounds are products of fermentation and in the gut serve as signals of microbial metabolism which are received by the gut epithelium and in turn used to coordinate anti-microbial homeostasis and epithelial microbial modulation. Similarly, TLR ligands are regulators of the immune response.

One action of NO is the nitrosylation of proteins. In particular tyrosine and cysteine side chains can be nitrosylated. Cysteine nitrosylation is often assisted by the donation of an NO group by nitrosylated glutathione. Glutathione nitrosylation is itself aided by molecular iron as a catalyst. The nitrosylation of cysteine in proteins like cysteine proteases renders them non-functional. Similarly, the nitrosylation of palmitoylation sites (cysteine clusters) in proteins like the Spike of SARS-CoV-2 also renders them non-functional because there cannot be a transfer from the thiol palmitoyl Co-A to the protein if the recipient cysteine is already blocked by a nitro group.

While all of these small natural modulators are known as extracellular signals, their use as intracellular modulators is not described. Here we report compounds that are designed to release these molecules in the cytoplasm and more importantly, acidic organelles such as the phagosome or lysosome. Release of these molecules in the lysosome serves to inform the cell that it has digested a bacterium and thereby induces an anti-bacterial program that in turn enables a more robust response to intracellular organisms that may otherwise suppress bacteriolysis.

In particular we describe molecules which are acid trapped and able to donate a compound that is the product of anaerobic metabolism. Acid trapped compounds are often amine containing compounds that are amphiphilic. They partition into the cell and concentrate in acidic compartments due to their conversion to an ionized form at pH 5-6 which is common in such organelles. Such acid-trapped molecules can be prepared with suitable linking groups such as hydroxyl groups. Multiple hydroxyl groups may be used to anchor one or more active molecules. There are many such acid-trapped compounds including common drugs such as propranolol, amodiaquine, dextromethorphan, Dextrorphan, paroxetine, fluoxetine, astemizole or imipramine. Another example is the macrolide class including compounds such as azithromycin, erythromycin or clarithromycin which are “acid trapped” in lysosomes by virtue of their 2′ amine groups and amphiphilic properties. Azithromycin has two amine groups and is particularly strongly trapped. These acid-trapped molecules can be derivatized, that is, decorated with signaling molecules related to anaerobic metabolism such as SCFAs, NO, or HS— donors to form compounds of the invention. Using multiple signaling molecules, or combinations thereof in multiple positions allows for a flexible means to tune the properties of the molecules. For example, we described different effects for a compound carrying 3 SCFA esters versus one carrying a NO ester and a SCFA or a thiol donor and an SCFA. In particular, there is a hierarchy of effect with longer fatty acids promoting differing immune responses. For example, propionate differs from acetate in the degree of effect in this setting.

Molecules of this type are able to influence the physiology of the lysosome and in particular its pH. In particular, acid trapped entities can raise endosomal and lysosomal pH such that they can impact processing of pathogens such as virus. The entities reported here may be used in combination with such pH modifying compounds at various ratios to increase their effects. They may also be combined with anti-viral compounds such as protease inhibitors and nucleoside analogs to provide combinations that both favourably impact the immune response to a virus while also inhibiting either viral or host processes related to viral proliferation.

### SUMMARY

The invention relates to compounds and combinations of compounds useful in treating or preventing infection, including disease or disorders associated with viral infection. The invention comprises compounds (e.g., derivative compounds of Amphiphilic Lysosomally trapped Compounds (ALC), such ALC compounds including those of the formulae and in any tables herein), which are useful alone or in combination with one more therapeutically useful agents in treating or preventing viral infection and disease or disorders associated with viral infection. The compounds and therapeutically useful agents can be used in pharmaceutical compositions and in methods of treating or preventing infection in a subject, including disease or disorders associated with viral infection in a subject.

The invention also relates to compounds useful in modulating immune cell activity or the barrier function of epithelial cells. The invention comprises compounds (e.g., derivative compounds of Amphiphilic Lysosomally trapped Compounds (ALC), such ALC compounds including those of the formulae in any tables herein), which are subject to lysosomal trapping and which bear moieties that are able to release TLR ligands, products of anaerobic metabolism, specifically SCFAs, sulfides, lactates, or NO, bile acids, polyamines, decarboxylated amino acids and polyphenol metabolites like phenylpropionic acid.

The invention also provides a method of identifying a compound useful for modulating immune cell activity against bacteria: incubating such a compound with blood cells, preferably leukocytes, providing those cells with bacteria, incubating the cells with bacteria, washing the cells and treating them with a non-permeable antibiotic to reduce extracellular bacteria, then counting intracellular bacteria to observe which compounds reduce the number of intracellular bacteria surviving.

Optionally, it is advantageous to determine the ratio of the concentration of the compound in the immune cells to non-immune cells such as erythrocytic cells as a measure of its lysosomal partition. Preferred are compounds that are preferentially taken up by immune cells.

In some embodiments the carrier compounds are macrolides with at least one ONO₂-, SNO₂- or NNO₂-moiety. In other embodiments the carrier compounds are macrolides with at least one SCFA-moiety. In other embodiments, the carrier molecule is amphiphilic with at least one protonatable amine. The term “macrolide” refers to any macrocyclic lactone with 10 or more atoms connected within the ring system. Reference to an atom includes all isotopes of that atom. For example, structures drawn with carbon or hydrogen include isotopes such as ¹³C or ²H.

An anti-microbial compound is a compound that inhibits the growth or division or replication of an organism such as a virus, bacteria, fungus, parasite, mycoplasma or other pathogen.

One embodiment is a compound comprising an Amphiphilic Lysosomally trapped Compound (ALC) conjugated via an ester, thioester or nitroester to a product of Anaerobic Metabolism (PAM) or one or more PAMs of the same or different types. In a further embodiment, the compound is one in which the PAM is selected from one or more of Short Chain Fatty Acid (SCFA), NO, H₂S, mercaptans, polyamines, decarboxylated amino acids or polyphenol metabolites like phenylpropionic acid. In a further embodiment, the compound is one in which the ALC is selected from a macrolide, polyamine, propranolol analog, chloroquine analog, amodiaquine, dextromethorphan, dextrorphan, paroxetine, fluoxetine, astemizole or imipramine analog.

Another embodiment is a macrolide comprising at least one ONO₂—, SNO₂— or NNO₂ moiety.

In some embodiments, the compound has the following formula (including any possible salts thereof, except for nitrates, and any structures with exchanged isotopes, as possible by state of the art):

### Formula 1

ALC conjugated or esterified with 1 or more of any of: X₍₁₋₅₎, Y₍₀₋₅₎, Z₍₀₋₃₎;

Where ALC=Amphiphilic Lysosomally trapped Compound;

X is a SCFA esterified to ALC and 0-5 indicates the number of moieties conjugated;

Y is an NO donating group or an H₂S donating group esterified to ALC and 0-5 indicates the number of moieties conjugated;

Z is a group donating sulfides, polyamines, decarboxylated amino acids or polyphenol metabolites like phenylpropionic acid.

### Formula 2: Compounds of Formula 1, Wherein ALC is a Macrolide of the Formula

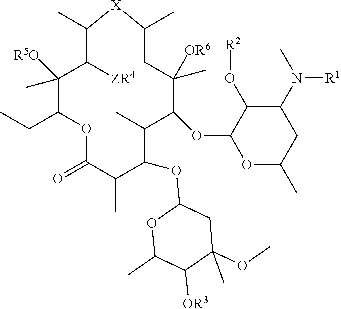

Wherein,

X═—N(CH₃)—CH₂—;
—CH₂—N(CH₃)—;
—C(═O)—;
—C(═NOR⁸)—;
—C(═NR¹²)—;

R¹ can be, but is not limited to
—(C₁-C₁₀)alkyl;
—(C₁-C₁₀)alkyliden-OH;
—(C₁-C₁₀)alkyliden-ONO₂;

R² can be, but is not limited to:
—H;
—NO_{(y)} with y=1 or 2;
—C(═O)OR⁷, —C(═S)OR⁷, —C(═O)R⁷, —C(═S)R⁷, —C(═O)(NH)R⁷, —C(═S)(NH)R⁷;

R³ can be, but is not limited to:
—H;
—NO_{(y)} with y=1 or 2;
—C(═O)OR⁷, —C(═S)OR⁷, —C(═O)R⁷, —C(═S)R⁷, —C(═O)(NH)R⁷, —C(═S)(NH)R⁷;

If Z═O, R⁴ can be, but is not limited to:
—H;
—NO_{(y)} with y=1 or 2;
—C(═O)OR⁷, —C(═S)OR⁷, —C(═O)R⁷, —C(═S)R⁷, —C(═O)(NH)R⁷, —C(═S)(NH)R⁷;

R₅ can be, but is not limited to:
—H;
—NO_{(y)} with y=1 or 2;
—C(═O)OR⁷, —C(═S)OR⁷, —C(═O)R⁷, —C(═S)R⁷, —C(═O)(NH)R⁷, —C(═S)(NH)R⁷;

or Z═O or NR⁹ and the R⁴ and R⁵ bearing atoms are connected via
—C(═O)— (If Z═O: carbonate linkage, if Z═NR⁹: carbamate linkage) or the R⁴ and R⁵ bearing atoms are connected via W;
   W may be but is not limited to
   —(−)CH—(C₁-C₁₂)alkyl;
   —(−)CH—(C₃-C₁₂)alkenyl;
   —(−)CH—(C₃-C₁₂)alkynyl;
   —(−)CH—(C₁-C₈)[(C₁-C₄)alkoxy]alkyl;
   —(−)CH—(C₁-C₈)[(C₁-C₄)alkoxy]alkenyl;
wherein alkyl, alkenyl, alkynyl are optionally substituted by one to five substituents selected independently from halogen (as can be F, Cl, Br, I), (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, —NR¹⁴R¹⁵, R¹⁴C(═O)—, R¹⁴C(═O)O—, R¹⁴OC(═O)O—, R¹⁴NHC(═O)—, R¹⁴C(═O)NH—, R¹⁴R¹⁵NC(═O)—, R¹⁴OC(═O)—, and -xNO₂ with x=O; S; N;

R⁶ can be, but is not limited to:
—H;
—NO_{(y)} with y=1 or 2;
—C(═O)OR⁷, —C(═S)OR⁷, —C(═O)R⁷, —C(═S)R⁷, —C(═O)(NH)R⁷, —C(═S)(NH)R⁷;

R⁷ can be independently chosen from:
—H;
—ferrocene;
—C₁-C₁₀ alkyl, alkenyl, alkynyl, aryl, heteroaryl, alkylaryl, alkylheteroaryl wherein alkyl, alkenyl, alkynyl, aryl, heteroaryl, alkylaryl and alkylheteroaryl groups are optionally substituted by one to five substituents selected independently from: ferrocene, halogen (as can be F, Cl, Br, I), (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxyl (—OH), nitro (—NO₂), cyano (—CN), azido (—N₃), mercapto (—SH), —NR¹⁴R¹⁵, R¹⁴C(═O)—, R¹⁴C(═O)O—, R⁴OC(═O)O—, R¹⁴NHC(═O)—, R¹⁴C(═O)NH—, R¹⁴R¹⁵NC(═O)—, R¹⁴OC(═O)—, and —XNO_{(y)} with X═O; S; N and y=1 or 2;

R⁸ can be, but is not limited to:
—H;
—NO_{(y)} with y=1 or 2;
—C(═O)—R⁷;
—(C₁-C₁₂)alkyl;
—(C₁-C₁₂)alkenyl;
—(C₁-C₁₂)alkynyl;
—(C₁-C₈)[(C₁-C₄)alkoxy]alkyl;
—(C₁-C₈)[(C₁-C₄)alkoxy]alkenyl;
—(C₆-C₁₀)aryl-(C₁-C₅)alkyl;
—(C₂-C₉)heteroaryl-(C₁-C₅)alkyl;

R⁹ can be, but is not limited to
—H;
—NO_{(y)} with y=1 or 2;
—C(═O)—R⁷;
—(C₁-C₁₂)alkyl;
—(C₁-C₁₂)alkenyl;
—(C₁-C₁₂)alkynyl;
—(C₁-C₈)[(C₁-C₄)alkoxy]alkyl;
—(C₁-C₈)[(C₁-C₄)alkoxy]alkenyl;
—(C₆-C₁₀)aryl-(C₁-C₅)alkyl;
—(C₂-C₉)heteroaryl-(C₁-C₅)alkyl;

R¹² can be, but is not limited to:
—H;
—NO_{(y)} with y=1 or 2;
—C(═O)—R⁷;
—(C₁-C₁₂)alkyl;
—(C₁-C₁₂)alkenyl;
—(C₁-C₁₂)alkynyl;
—(C₁-C₈)[(C₁-C₄)alkoxy]alkyl;
—(C₁-C₈)[(C₁-C₄)alkoxy]alkenyl;
—(C₆-C₁₀)aryl-(C₁-C₅)alkyl;
—(C₂-C₉)heteroaryl-(C₁-C₅)alkyl;

R¹⁴, R¹⁵ can independently be, but are not limited to:
—H;
—(C₁-C₁₂)alkyl;
—(C₁-C₁₂)alkenyl;
—(C₁-C₁₂)alkynyl;
—(C₁-C₈)[(C₁-C₄)alkoxy]alkyl;
—(C₁-C₈)[(C₁-C₄)alkoxy]alkenyl;
—(C₆-C₁₀)aryl-(C₁-C₅)alkyl;
—(C₂-C₉)heteroaryl-(C₁-C₅)alkyl
wherein alkyl, alkenyl, alkynyl, aryl and heteroaryl are optionally substituted by one to five substituents selected independently from halogen (as can be F, Cl, Br, I), (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxyl (—OH), nitro (—NO₂), cyano (—CN), azido (—N3), mercapto (—SH), and —XNO_{y} with X═O; S; N and y=1 or 2; or N(R¹⁴R¹⁵) is an aziridine, azetidine, pyrrolidine, piperidine, azepane or azocane, 1-substituted piperazine, or morpholine moiety.

In some embodiments, the compound has the following formula (including any possible salts thereof, except for nitrates, and any structures with exchanged isotopes, as possible by state of the art):

### Formula 1

ALC conjugated or esterified with 1 or more of any of: X₍₁₋₅₎, Y₍₀₋₅₎, Z₍₀₋₃₎;

Where ALC=Amphiphilic Lysosomally trapped Compound;

X is a SCFA esterified to ALC and 0-5 indicates the number of moieties conjugated;

Y is an NO donating group or an H₂S donating group esterified to ALC and 0-5 indicates the number of moieties conjugated;

Z is a group donating sulfides, polyamines, decarboxylated amino acids or polyphenol metabolites like phenylpropionic acid.

### Formula 2: Compounds of Formula 1, Wherein ALC is a Macrolide of the Formula

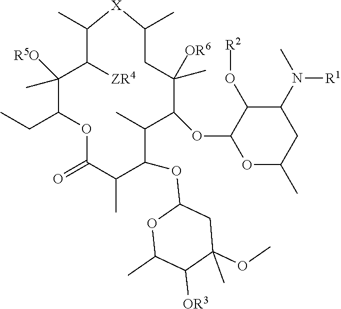

Wherein,

X═—N(CH₃)—CH₂—;
—CH₂—N[(CH₂)ₙCH₃]—; wherein n is 0-4;
—C(═O)—;
—C(═NOR⁸)—;
—C(═NR¹²)—;

R¹ can be, but is not limited to
—(C₁-C₁₀)alkyl;
—(C₁-C₁₀)alkyliden-OH;
—(C₁-C₁₀)alkyliden-ONO₂;

R² can be, but is not limited to:
—H;
—NO_{(y)} with y=1 or 2;
—C(═O)OR⁷, —C(═S)OR⁷, —C(═O)R⁷, —C(═S)R⁷, —C(═O)(NH)R⁷, —C(═S)(NH)R⁷;

R³ can be, but is not limited to:
—H;
—NO_{(y)} with y=1 or 2;
—C(═O)OR⁷, —C(═S)OR⁷, —C(═O)R⁷, —C(═S)R⁷, —C(═O)(NH)R⁷, —C(═S)(NH)R⁷;

If Z═O, R⁴ can be, but is not limited to:
—H;
—NO_{(y)} with y=1 or 2;
—C(═O)OR⁷, —C(═S)OR⁷, —C(═O)R⁷, —C(═S)R⁷, —C(═O)(NH)R⁷, —C(═S)(NH)R⁷;

R⁵ can be, but is not limited to:
—H;
—NO_{(y)} with y=1 or 2;
—C(═O)OR⁷, —C(═S)OR⁷, —C(═O)R⁷, —C(═S)R⁷, —C(═O)(NH)R⁷, —C(═S)(NH)R⁷;

or Z═O or NR⁹ and the R⁴ and R⁵ bearing atoms are connected via
—C(═O)— (If Z═O: carbonate linkage, if Z═NR⁹: carbamate linkage) or the R⁴ and R⁵ bearing atoms are connected via W;
   W may be but is not limited to:
   —(−)CH—(C₁-C₁₂)alkyl;
   —(−)CH—(C₃-C₁₂)alkenyl;
   —(−)CH—(C₃-C₁₂)alkynyl;
   —(−)CH—(C₁-C₈)[(C₁-C₄)alkoxy]alkyl;
   —(−)CH—(C₁-C₈)[(C₁-C₄)alkoxy]alkenyl;
wherein alkyl, alkenyl, alkynyl are optionally substituted by one to five substituents selected independently from halogen (as can be F, Cl, Br, I), (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, —NR¹⁴R¹⁵, R¹⁴C(═O)—, R¹⁴C(═O)O—, R¹⁴OC(═O)O—, R¹⁴NHC(═O)—, R¹⁴C(═O)NH—, R¹⁴R¹⁵NC(═O)—, R¹⁴OC(═O)—, and -xNO₂ with x=O; S; N;

R⁶ can be, but is not limited to:
—H;
—NO_{(y)} with y=1 or 2;
—C(═O)OR⁷, —C(═S)OR⁷, —C(═O)R⁷, —C(═S)R⁷, —C(═O)(NH)R⁷, —C(═S)(NH)R⁷;

R⁷ can be independently chosen from:
—H;
—ferrocene;
—C₁-C₁₀ alkyl, alkenyl, alkynyl, aryl, heteroaryl, alkylaryl, alkylheteroaryl wherein alkyl, alkenyl, alkynyl, aryl, heteroaryl, alkylaryl and alkylheteroaryl groups are optionally substituted by one to five substituents selected independently from: ferrocene, halogen (as can be F, Cl, Br, I), (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxyl (—OH), nitro (—NO₂), cyano (—CN), azido (—N3), mercapto (—SH), —NR¹⁴R¹⁵, R¹⁴C(═O)—, R¹⁴C(═O)O—, R¹⁴OC(═O)O—, R¹⁴NHC(═O)—, R¹⁴C(═O)NH—, R¹⁴R¹⁵NC(═O)—, R¹⁴OC(═O)—, and —XNO_{(y)} with X═O; S; N and y=1 or 2;

R⁸ can be, but is not limited to:
—H;
—NO_{(y)} with y=1 or 2;
—C(═O)—R⁷;
—(C₁-C₁₂)alkyl;
—(C₁-C₁₂)alkenyl;
—(C₁-C₁₂)alkynyl;
—(C₁-C₈)[(C₁-C₄)alkoxy]alkyl;
—(C₁-C₈)[(C₁-C₄)alkoxy]alkenyl;
—(C₆-C₁₀)aryl-(C₁-C₅)alkyl;
—(C₂-C₉)heteroaryl-(C₁-C₅)alkyl;

R⁹ can be, but is not limited to
—H;
—NO_{(y)} with y=1 or 2;
—C(═O)—R⁷;
—(C₁-C₁₂)alkyl;
—(C₁-C₁₂)alkenyl;
—(C₁-C₁₂)alkynyl;
—(C₁-C₈)[(C₁-C₄)alkoxy]alkyl;
—(C₁-C₈)[(C₁-C₄)alkoxy]alkenyl;
—(C₆-C₁₀)aryl-(C₁-C₅)alkyl;
—(C₂-C₉)heteroaryl-(C₁-C₅)alkyl;

R¹² can be, but is not limited to:
—H;
—NO_{(y)} with y=1 or 2;
—C(═O)—R⁷;
—(C₁-C₁₂)alkyl;
—(C₁-C₁₂)alkenyl;
—(C₁-C₁₂)alkynyl;
—(C₁-C₈)[(C₁-C₄)alkoxy]alkyl;
—(C₁-C₈)[(C₁-C₄)alkoxy]alkenyl;
—(C₆-C₁₀)aryl-(C₁-C₅)alkyl;
—(C₂-C₉)heteroaryl-(C₁-C₅)alkyl;

R¹⁴, R¹⁵ can independently be, but are not limited to:
—H;
—(C₁-C₁₂)alkyl;
—(C₁-C₁₂)alkenyl;
—(C₁-C₁₂)alkynyl;
—(C₁-C₈)[(C₁-C₄)alkoxy]alkyl;
—(C₁-C₈)[(C₁-C₄)alkoxy]alkenyl;
—(C₆-C₁₀)aryl-(C₁-C₅)alkyl;
—(C₂-C₉)heteroaryl-(C₁-C₅)alkyl;
wherein alkyl, alkenyl, alkynyl, aryl and heteroaryl are optionally substituted by one to five substituents selected independently from halogen (as can be F, Cl, Br, I), (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxyl (—OH), nitro (—NO₂), cyano (—CN), azido (—N3), mercapto (—SH), and —XNO_{y} with X═O; S; N and y=1 or 2 or N(R¹⁴R¹⁵) is an aziridine, azetidine, pyrrolidine, piperidine, azepane or azocane, 1-substituted piperazine, or morpholine moiety.

In another aspect, the compounds are of Formula 2 above, wherein

R¹ is:
—H
—(C₁-C₁₀)alkyl;
—(C₁-C₁₀)alkyliden-OH; or
—(C₁-C₁₀)alkyliden-ONO₂; and

the remaining variables in Formula 2 are as described above.

In some other embodiments, the compound has the following formula (including any possible salts thereof, except for nitrates, and any structures with exchanged isotopes, as possible by state of the art):

### Formula 3′: Compounds of Formula 1, Wherein ALC is a Macrolide of the Formula

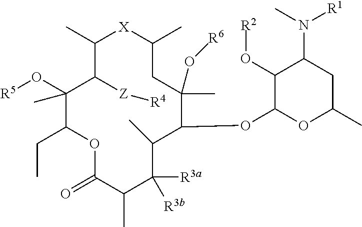

Wherein R¹, R², R⁴, R⁵, R⁶, X, and Z are defined as in formula 2;
R^{3a}, R^{3b}=both —H;
or in the case R^{3a} is —H, R^{3b} can be:
—OH;
—OR¹⁴;
—NR¹⁴R¹⁵;
⁻C(═O)—R⁷;
   or R^{3a}=R^{3b}=(═O);
=any possible cyclic or non-cyclic acetal;
(═NR¹²);
=any possible cyclic or non-cyclic aminal;
—OC(═O)R⁷;
OR¹⁴;
   and R⁷, R¹⁴, and R¹⁵ are defined as in formula 2.

### Formula 3: Compounds of Formula 1, Wherein ALC is a Macrolide of the Formula

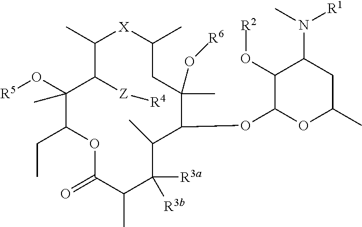

Wherein R¹, R², R⁴, R⁵, R⁶, X, and Z are defined as in formula 2;
R^{3a}, R^{3b}=both —H;
or in the case R^{3a} is —H, R^{3b} can be:
—OH;
—OR¹⁴;
—O-glycoside
—NR¹⁴R¹⁵;
⁻C(═O)—R⁷;
   wherein glycoside can be a glycosidic bound monosaccaride, for example, but not being limited to mannose, fucose, arabinose, xylose, the monosaccharides being free or with hydroxy groups protected as acetate or propionate;
   or R^{3a}=R^{3b}=(═O);
=any possible cyclic or non-cyclic acetal;
(═NR¹²);
=any possible cyclic or non-cyclic aminal;
—OC(═O)R⁷;
OR¹⁴;
   and R⁷, R¹⁴, and R¹⁵ are defined as in formula 2.

### Formula 4: Compounds of Formula 1, Wherein ALC is a Propranolol of the Formula

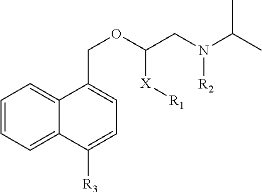

Where X can be O or S;

When X═O, R₁ may be but not limited to —(C═O)CH₃, —(C═O)CH₂CH₃, —(C═O)CH₂CH₂CH₃, —(C═O)CH₂CH₂COOH, —(C═O)(C═O)CH₃, —(C═O)CHCHCOOH, —(C═O)CH(OH)CH₃, —(C═O)C(CH₃)₂, —(C═O)CH₂CH₂CH₂CH₃, —(C═O)CH₂C(CH₃)₂, —(C═O)CH₂CH₂CH₂CH₂Y, or —(C═O)CH(ONO₂)CH₃;

R₂═R₃═H;

Y=can be a 5-membered saturated ring containing a disulfide bond;

When X═O, R₁ may be but not limited to —(C═O)CH₃, —(C═O)CH₂CH₃, —(C═O)CH₂CH₂CH₃, —(C═O)CH₂CH₂COOH, —(C═O)(C═O)CH₃, —(C═O)CHCHCOOH, —(C═O)CH(OH)CH₃, —(C═O)C(CH₃)₂, —(C═O)CH₂CH₂CH₂CH₃, —(C═O)CH₂C(CH₃)₂, —(C═O)CH₂CH₂CH₂CH₂Y, or —(C═O)CH(ONO₂)CH₃;

R₂═CH₃; R₃═H; or

When X═O, R₁═NO₂; R² consists of linker —CH₂CH₂OR₄, where R⁴ may be but not limited to —(C═O)CH₃, —(C═O)CH₂CH₃, —(C═O)CH₂CH₂CH₃, —(C═O)CH₂CH₂COOH, —(C═O)(C═O)CH₃, —(C═O)CHCHCOOH, —(C═O)CH(OH)CH₃, —(C═O)C(CH₃)₂, —(C═O)CH₂CH₂CH₂CH₃, —(C═O)CH₂C(CH₃)₂, or —(C═O)CH₂CH₂CH₂CH₂Y; R3═H;

Y=can be a 5-membered saturated ring containing a disulfide bond; or

When X═O, R₁ may be but not limited to —(C═O)CH₃, —(C═O)CH₂CH₃, —(C═O)CH₂CH₂CH₃, —(C═O)CH₂CH₂COOH, —(C═O)(C═O)CH₃, —(C═O)CHCHCOOH, —(C═O)CH(OH)CH₃, —(C═O)C(CH₃)₂, —(C═O)CH₂CH₂CH₂CH₃, —(C═O)CH₂C(CH₃)₂, —(C═O)CH₂CH₂CH₂CH₂Y, or —(C═O)CH(ONO₂)CH₃;

Y=can be a 5-membered saturated ring containing a disulfide bond;

R² consists of linker —CH₂CH₂OR₄, where R₄═NO₂; R₃═H; or

When X═O, R₁ is NO₂, R₂═H or CH₃, R₃═OR₅, where R₅ may be but not limited to —(C═O)CH₃, —(C═O)CH₂CH₃, —(C═O)CH₂CH₂CH₃, —(C═O)CH₂CH₂COOH, —(C═O)(C═O)CH₃, —(C═O)CHCHCOOH, —(C═O)CH(OH)CH₃, —(C═O)C(CH₃)₂, —(C═O)CH₂CH₂CH₂CH₃, —(C═O)CH₂C(CH₃)₂, —(C═O)CH₂CH₂CH₂CH₂Y, or —(C═O)CH(ONO₂)CH₃;

Y=can be a 5-membered saturated ring containing a disulfide bond; or

When X═S, R¹ may be but not limited to —(C═O)CH₃, a metal salt, or forms a disulfide bridge with itself, R²═R³═H.

Definition of substituents on ALC formulae (e.g., Macrolides, hydroxychloroquine, Propranolol, etc.)

| | |
|---|---|
| —NO₂ | Nitro |
| —(C═O)—C₆H₄—NO₂ | p-nitrobenzoyl |
| —(C═O)C₆H₅ | benzoyl |
| —(C═O)CH₂CH₂COOH | succinyl |
| —(C═O)CH₂CH₃ | propionyl |
| —(C═O)CH₂CH₂CH₃ | butyryl |
| —(C═O)CH₃ | acetyl |
| —(C═O)(C═O)CH₃ | Pyruvyl |
| —(C═O)CHCHCOOH | Maleyl |
| —(C═O)CH(OH)CH₃ | Lactyl |
| —(C═O)CH(ONO₂)CH₃ | 2-O-Nitrolactyl |
| —(C═O)C(CH₃)₂ | Isobutyryl |
| —(C═O)CH₂CH₂CH₂CH₃ | Valeryl |
| —(C═O)CH₂C(CH₃)₂ | Isovalericyl |
| —(C═O)CH(CH₃)O(C═O)CH₃ | Acetoxypropionyl |
| —(C═O)CH₂CH₂(C═O)—Z | Succinyl-dithiole-3-thione |
| —(C═O)OCH₂CH₂S(S)ₙH | Polysulfide ethyl carbonate |
| —(C═O)OCH₂CH₂SNO | NO-thioethylcarbonate |
| —(C═S)OC₆H₅ | O-Phenylchlorothiono carbonate |
| —(C═O)CH₂CH₂CH₂CH₂CH₃ | hexanoyl |
| —CH₂CH₂Br | bromoethyl |
| —(C═O)OCH₂CHCH₂ | Vinyl carbonate |
| —(C═O)CH₂CH₂CH₂CH₂Y | Lipoyl |

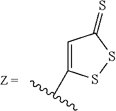 [SMILES: *c1cc(=S)ss1]

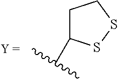 [SMILES: *C1CCSS1]

Formula 5: Compounds with the Structure

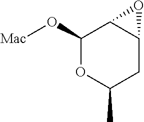

Wherein

Mac=a macrolide ring or macrolide ring system, for example, but not limited to azithromycin or gamithromycin, each without the desosamin residue.

Compounds with the Structure

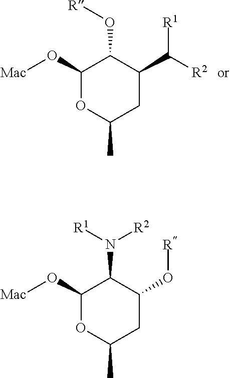

Wherein

Mac=a macrolide ring or macrolide ring system, for example, but not limited to azithromycin or gamithromycin, each without the desosamin residue;
R″=independently of each other

—H;

—NO_{(y)} with y=1 or 2;

—C(═O)OR³, —C(═S)OR³, —C(═O)R³, —C(═S)R³, —C(═O)(NH)R³, —C(═S)(NH)R³;

R¹, R²=independently of each other H, OH, OR⁴, —C₁-C₁₀ alkyl, alkenyl, alkynyl, aryl, heteroaryl, alkylaryl, alkylheteroaryl;

wherein alkyl, alkenyl, alkynyl, aryl, heteroaryl, alkylaryl and alkylheteroaryl groups are optionally substituted by one to five substituents selected independently from: fluorine, (C₁-C₄)alkyl, (C₁-C₄)alkenyl, (C₁-C₄)alkynyl, (C₃-C₇)cycloalkyl, (C₁-C₉)heterocycloalkyl, (C₆-C₁₀)aryl, (C₁-C₉)heteroaryl, (C₁-C₄)alkoxy, hydroxyl (—OH), nitro (—NO₂), cyano (—CN), azido (—N₃), mercapto (—SH), (C₁-C₄)alkthio, —NR⁴R⁵, R⁴C(═O)—, R⁴C(═O)O—, R⁴OC(═O)O—, R⁴NHC(═O)—, R⁴C(═O)NH—, R⁴R⁵NC(═O)—, R⁴OC(═O)—, and —XNO_{(y)} with X═O; S; N and y=1 or 2;

or N(R¹R²) is an aziridine, azetidine, pyrrolidine, piperidine, azepane or azocane, 1-substituted piperazine, or morpholine moiety;

R³═—C₁-C₁₀ alkyl, alkenyl, alkynyl, aryl, heteroaryl, alkylaryl, alkylheteroaryl wherein alkyl, alkenyl, alkynyl, aryl, heteroaryl, alkylaryl and alkylheteroaryl groups are optionally substituted by one to five substituents selected independently from: halogen, (C₁-C₄)alkyl, (C₁-C₄)alkenyl, (C₁-C₄)alkynyl, (C₃-C₇)cycloalkyl, (C₁-C₉)heterocycloalkyl, (C₆-C₁₀)aryl, (C₁-C₉)heteroaryl, (C₁-C₄)alkoxy, hydroxyl (—OH), nitro (—NO₂), cyano (—CN), azido (—N₃), mercapto (—SH), (C₁-C₄)alkthio, —NR⁶R⁷, R⁶C(═O)—, R⁶C(═O)O—, R⁶OC(═O)O—, R⁶NHC(═O)—, R⁶C(═O)NH—, R⁶R⁷NC(═O)—, R⁶OC(═O)—, and —XNO_{(y)} with X═O; S; N and y=1 or 2;

R⁴, R⁵, R⁶ and R⁷ can independently be, but are not limited to:
—H;
—(C₁-C₁₂)alkyl;
—(C₁-C₁₂)alkenyl;
—(C₁-C₁₂)alkynyl;
—(C₁-C₈)[(C₁-C₄)alkoxy]alkyl;
—(C₁-C₈)[(C₁-C₄)alkoxy]alkenyl;
—(C₆-C₁₀)aryl-(C₁-C₅)alkyl;
—(C₂-C₉)heteroaryl-(C₁-C₅)alkyl;
   wherein alkyl, alkenyl, alkynyl, aryl and heteroaryl are optionally substituted by one to five substituents selected independently from ferrocene, halogen (as can be F, Cl, Br, I), (C₁-C₄)alkyl, (C₁-C₄)alkenyl, (C₁-C₄)alkynyl, (C₃-C₇)cycloalkyl, (C₁-C₉)heterocycloalkyl, (C₆-C₁₀)aryl, (C₁-C₉)heteroaryl, (C₁-C₄)alkoxy, hydroxyl (—OH), (C₁-C₆)acyloxy, nitro (—NO₂), cyano (—CN), azido (—N₃), mercapto (—SH), and —XNO_{y} with X═O; S; N and y=1 or 2.

Compounds with the Structure

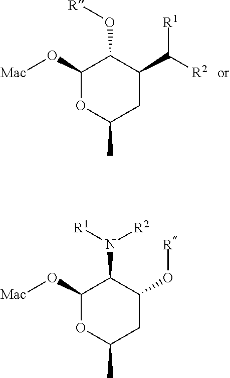

Wherein

Mac=a macrolide ring or macrolide ring system, for example, but not limited to azithromycin or gamithromycin, each with the desosamin residue being substituted by above structures;
R″=independently of each other

—H;

—NO_{(y)} with y=1 or 2;

—C(═O)OR³, —C(═S)OR³, —C(═O)R³, —C(═S)R³, —C(═O)(NH)R³, —C(═S)(NH)R³;

R¹, R²=independently of each other H, OH, OR⁴, —C₁-C₁₀ alkyl, alkenyl, alkynyl, aryl, heteroaryl, alkylaryl, alkylheteroaryl;

wherein alkyl, alkenyl, alkynyl, aryl, heteroaryl, alkylaryl and alkylheteroaryl groups are optionally substituted by one to five substituents selected independently from: fluorine, (C₁-C₄)alkyl, (C₁-C₄)alkenyl, (C₁-C₄)alkynyl, (C₃-C₇)cycloalkyl, (C₁-C₉)heterocycloalkyl, (C₆-C₁₀)aryl, (C₁-C₉)heteroaryl, (C₁-C₄)alkoxy, hydroxyl (—OH), nitro (—NO₂), cyano (—CN), azido (—N₃), mercapto (—SH), (C₁-C₄)alkthio, —NR⁴R⁵, R⁴C(═O)—, R⁴C(═O)O—, R⁴OC(═O)O—, R⁴NHC(═O)—, R⁴C(═O)NH—, R⁴R⁵NC(═O)—, R⁴OC(═O)—, and —XNO_{(y)} with X═O; S; N and y=1 or 2;

or N(R¹R²) is an aziridine, azetidine, pyrrolidine, piperidine, azepane or azocane, 1-substituted piperazine, or morpholine moiety;

R³═—C₁-C₁₀ alkyl, alkenyl, alkynyl, aryl, heteroaryl, alkylaryl, alkylheteroaryl wherein alkyl, alkenyl, alkynyl, aryl, heteroaryl, alkylaryl and alkylheteroaryl groups are optionally substituted by one to five substituents selected independently from: halogen, (C₁-C₄)alkyl, (C₁-C₄)alkenyl, (C₁-C₄)alkynyl, (C₃-C₇)cycloalkyl, (C₁-C₉)heterocycloalkyl, (C₆-C₁₀)aryl, (C₁-C₉)heteroaryl, (C₁-C₄)alkoxy, hydroxyl (—OH), nitro (—NO₂), cyano (—CN), azido (—N₃), mercapto (—SH), (C₁-C₄)alkthio, —NR⁶R⁷, R⁶C(═O)—, R⁶C(═O)O—, R⁶OC(═O)O—, R⁶NHC(═O)—, R⁶C(═O)NH—, R⁶R⁷NC(═O)—, R⁶OC(═O)—, and —XNO_{(y)} with X═O; S; N and y=1 or 2;

R⁴, R⁵, R⁶ and R⁷ can independently be, but are not limited to:
—H;
—(C₁-C₁₂)alkyl;
—(C₁-C₁₂)alkenyl;
—(C₁-C₁₂)alkynyl;
—(C₁-C₈)[(C₁-C₄)alkoxy]alkyl;
—(C₁-C₈)[(C₁-C₄)alkoxy]alkenyl;
—(C₆-C₁₀)aryl-(C₁-C₅)alkyl;
—(C₂-C₉)heteroaryl-(C₁-C₅)alkyl;
   wherein alkyl, alkenyl, alkynyl, aryl and heteroaryl are optionally substituted by one to five substituents selected independently from ferrocene, halogen (as can be F, Cl, Br, I), (C₁-C₄)alkyl, (C₁-C₄)alkenyl, (C₁-C₄)alkynyl, (C₃-C₇)cycloalkyl, (C₁-C₉)heterocycloalkyl, (C₆-C₁₀)aryl, (C₁-C₉)heteroaryl, (C₁-C₄)alkoxy, hydroxyl (—OH), (C₁-C₆)acyloxy, nitro (—NO₂), cyano (—CN), azido (—N₃), mercapto (—SH), and —XNO_{y} with X═O; S; N and y=1 or 2.

Unless, otherwise stated, the word “macrolide” herein refers to the family of well-known macrocyclic lactone antibiotics and also the various macrocyclic lactone ALCs described herein.

In one aspect, the invention provides a composition comprising a compound of any of the formulae herein (e.g., any of the formulae, any formula in the tables herein), or salt, solvate, hydrate or prodrug thereof, and a pharmaceutically acceptable carrier. In a further aspect, the composition can further comprise an additional therapeutic agent.

In one aspect, the invention provides a method of treating a subject suffering from or susceptible to a disease, disorder, or symptom thereof. The method includes administering to a subject in need thereof a therapeutically effective amount of a compound of any of the formulae herein (e.g., any of the formulae, any formula in the tables herein), or salt, solvate, hydrate or prodrug thereof. The disease, disorder, or symptom thereof can be, for example, an infectious disease, an inflammatory disease, a malignant disease, a bacterial infection, an inflammatory reaction to a bacterial translocation event, an inflammation of the GI tract including intestines, colon, liver and pancreas, an inflammation of the airways, a systemic inflammatory disease or a malignant or neoplastic disease.

In one aspect, the invention provides a method of stimulating immune or epithelial cells to form an anti-infective barrier or anti-infective response comprising contacting the cells with a compound of any of the formulae herein (e.g., any of the formulae, any formula in the tables herein), or salt, solvate, hydrate or prodrug thereof. A further aspect of the method is wherein the contacting results in the intracellular release of a PAM comprising one or more of a molecule type selected from SCFA, NO, H₂S, sulfides, polyamines, decarboxylated amino acids or polyphenol metabolites like phenylpropionic acid from the compound of any of the formulae herein (e.g., any of the formulae, any formula in the tables herein), or salt, solvate, hydrate or prodrug thereof. A further aspect of the method is that comprising the intracellular release of one or more types of a short chain fatty acid moiety from the compound of any of the formulae herein (e.g., any of the formulae, any formula in the tables herein), or salt, solvate, hydrate or prodrug thereof. A further aspect of the method is that comprising the intracellular release of a short chain fatty acid moiety containing 2 or more carbons from an appropriate carrier molecule.

In one embodiment, an ALC compound is a compound such as azithromycin or hydroxychloroquine or similar anti-infective compound. The ALC compound in its unconjugated state is physically mixed with the compounds of Formulas 1, 2 or 3 at ratios from 1:1 to 1000:1 (ALC:derivative in Formula 1, 2 or 3). The ALC compound can also be mixed with an anti-viral compound such as serine protease inhibitors, (e.g., camostat), protease inhibitors or nucleoside analog. In certain embodiments, the mixture is further mixed with zinc orotate or taken simultaneously with zinc orotate at a dose between 5 and 40 mg of elemental zinc equivalent. These embodiments may be useful in treating viral diseases, especially those causing viral pneumonia.

In one aspect, such mixtures can be used to treat pneumonias associated with influenza, coronaviridae including MERS-CoV, SARS-CoV and SARS-CoV-2, respiratory syncytial virus (RSV), human parainfluenza viruses, adenoviruses, metapneumovirus, or hantaviruses. In another aspect, such mixtures can be used to treat viral pneumonias complicated by bacterial infections.

In one aspect, such mixtures can be used to treat infections associated with flavivirus types, dengue, Zika, HIV, herpes, EBV, rotavirus, Hepatitis A, B, C, E, influenza, coronaviridae including MERS-CoV, SARS-CoV and SARS-CoV-2, respiratory syncytial virus (RSV), human parainfluenza viruses, adenoviruses, metapneumovirus, or hantaviruses. In another aspect, such mixtures can be used to treat viral pneumonias complicated by bacterial infections. In another aspect, such mixtures can be used to treat viral infections complicated by bacterial infections.

In an embodiment, the compound of the invention is administered to the subject using a pharmaceutically-acceptable formulation, e.g., a pharmaceutically-acceptable formulation that provides sustained delivery of the compound of the invention to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compounds of the present inventions, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, mannitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present. Solubilizing agents, including for example, cremaphore and beta-cyclodextrins can also be used in the pharmaceutical compositions herein.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, (e.g., 0.01 to 1 mg/kg effective dose) but can be higher or lower, depending upon, among other factors, the activity of the compound, its bioavailability, the mode of administration, and various factors discussed above. Dosage amount and interval can be adjusted individually to provide plasma levels of the compound(s) which are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation

The compound(s) can be administered once per day, a few or several times per day, or even multiple times per day, depending upon, among other things, the indication being treated and the judgment of the prescribing physician.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

Another object of the present invention is the use of a compound as described herein (e.g., of any formulae herein) in the manufacture of a medicament for use in the treatment of a disorder or disease herein. Another object of the present invention is the use of a compound as described herein (e.g., of any formulae herein) for use in the treatment of a disorder or disease herein.

Many compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms.

Further, the aforementioned compounds also include their N-oxides. The term “N-oxides” refers to one or more nitrogen atoms, when present in a compound, are in N-oxide form, i.e., N→O.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., treating a disease).

In another aspect the present invention relates to a mild and highly selective process for the in situ introduction of the O-, S- and N-nitrate group into compounds of any other formulae herein.

### DETAILED DESCRIPTION OF THE INVENTION

### Technical Problem

NO, SCFA or PAM are difficult to use directly in Pharmaceutical compositions.

The use of NO donors is well known in medicine as a means to modulate blood pressure and inflammation^{[13]}. Such NO donors exert their action, as aforementioned, largely in the extracellular environment. In the case of blood pressure regulators, release of NO in the blood results in effects on the endothelium which are desirable. In the case of modulators of inflammation, general release of NO may result in side effects such as loss of blood pressure which are undesirable.

Similarly, the SCFAs are volatile, malodourous and unstable. PAMs such as polyamines are similarly unattractive for direct use. While these products such as SCFAs or NO (inhaled gas) have been applied to the body, they are used in amounts that are undesirable. The technical problem to solve, was, therefore, to focus the release of these products (NO, SCFA, PAMs) to cells associated with inflammation, cancer or infection such that lower amounts could be used.

### Solution to Problem

The compounds reported here are mostly located in intracellular compartments and thus may donate NO or SCFA or PAM to an intracellular receptor, preferably an intra-phagosomal or lysosomal receptor. Macrolide anti-bacterial compounds are well known for their ability to be concentrated in acidic compartments, notably the phagosomes of immune cells such as neutrophils and macrophage^{[14]}. Phagosomes are the organs where bacteria and other debris are digested by the phagocytes using oxidative processes and digestive enzymes. Certain bacteria resist this process by reducing the capacity of the cell to produce antibacterial factors (lower pH, proteases, active oxygen species, antibacterial enzymes, NO).

If, however, a compound was also trapped in the phagosome that was capable of donating a stimulatory factor such as SCFA, PAM or NO, then there is the potential to overcome the inhibition due to the bacterium. More importantly, if the Phagocyte absorbs a compound able to donate SCFA, PAM or NO prior to that phagocyte encountering bacteria, it is potentially stimulated to better kill bacteria immediately on contact with them. This is potentially of significance in treating infections by bacteria such as Legionella, Pasteurella, Listeria and Mycobacterium species that are intracellular parasites. It is also potentially significant in the stimulation of barrier cells to resist the effect of bacteria, or to maintain physical barriers toward bacteria.

In addition to their roles in immunology, SCFA, NO and PAM have a role in homeostasis, acute inflammation and wound healing^{[15]}. Phagocytes like macrophages are involved in many aspects of metabolism and are sensitive to SCFA, NO and PAM. The delivery of these substances preferentially to cells of this type is a means to allow them to respond to the stimulus of SCFA, PAM or NO without using high systemic levels. This is achieved by delivering the substances as conjugates to lysosomally tropic compounds (ALCs).

Thus, the efficacy of molecules described herein in various models of inflammation and resolution of inflammation were examined. In these models, example compounds reported here were able to reduce the effects of inflammation, support body weight maintenance, and reduce disease signs without causing appreciable toxicity.

### Definitions

“Anti-infective barrier” means the ability of epithelium to prevent the penetration of bacteria or other pathogens.

Stimulating the formation of an anti-infective barrier means increasing the ability of epithelium to prevent the penetration of bacteria or other pathogens through the up-regulation of tight junction formation or other barrier functions.

“Anti-infective response” means the ability of immune cells or similar to prevent the growth of bacteria or other pathogens via phagocytosis, oxidative burst or other toxic responses inactivating the pathogen.

Stimulating the formation of an anti-infective response means increasing the ability of immune cells or similar cells to prevent the growth of bacteria or other pathogens via phagocytosis, oxidative burst or other toxic responses inactivating the pathogen.

“ALC” means an Amphiphilic Lysosomally trapped Compound.

“PAM” means Product of Anaerobic Metabolism. PAMs include but are not limited to SCFA, NO, H₂S, mercaptans that eventually generate H₂S/HS⁻ polyamines (e.g., compounds of Table 7), amino acid residues lacking the C-terminus (decarboxlated), bile acids (e.g., steroid acids found in the bile of mammals and other vertebrates, such as chenodeoxycholic acid, cholic acid, deoxycholic acid, lithocholic acid, and the like), or degradation products from polyphenol metabolism such as 3-(3-Hydroxyphenyl)propanoic (hMPP) acid, SCFA means Short Chain Fatty Acid, which is a fatty acid molecule having an aliphatic tail of eight or less carbon atoms.

“NO” means Nitric Oxide.

### Advantageous Effects of Invention

The compounds reported here are useful in many respects. They are anti-microbial, anti-inflammatory, able to accumulate in tumors and donate NO and able to protect against inflammation of the intestine. Selected embodiments are able to modulate inflammation of the liver and protect against accumulation of fat or the resulting fibrosis.

The compounds are readily soluble as salts, may be provided by the oral route, or via other means. They are adequately stable for pharmacological use when stored at the appropriate pH conditions.

### BRIEF DESCRIPTION OF DRAWINGS

FIG. 1: TNFa—Production by LPS treated mice with either Vehicle (1% citric acid in water, 5 mL/kg) or 10 μmol/kg Compound E2 or Compound E3.

FIG. 2: Change in body weight in mice in which arthritis has been induced using bovine collagen. Animals were treated with either Vehicle (1% citric acid in water, 5 mL/kg) or the indicated doses of compound E2 μmol/kg. Data are from 10 animals per group, and data points significant different from Vehicle are marked with *.

FIG. 3: Number of mice maintaining body weight in which intestinal inflammation has been induced using Dextran Sulfate. Mice were treated with Vehicle (1% citric acid in water, 5 mL/kg) or the indicated doses of compound E2 in μmol/kg.

FIG. 4: Killing of phagocytosed Salmonella typhimurium by murine macrophages treated with either the commercial antibiotic azithromycin or Compound E2. Compound E2 stimulates the killing of bacterial cells by macrophages.

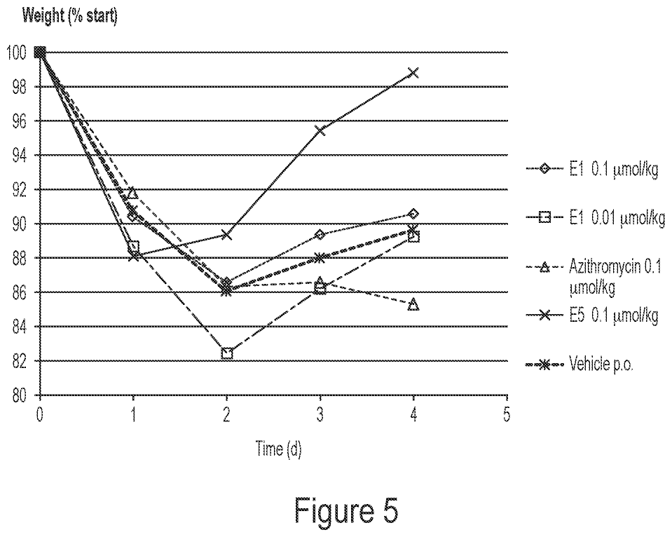

FIG. 5: Effect of substance E5 on the response of mice to an infection by Staphylococcus. Treatment with the substance E5 results in a faster recovery of weight due to faster clearance of bacteria

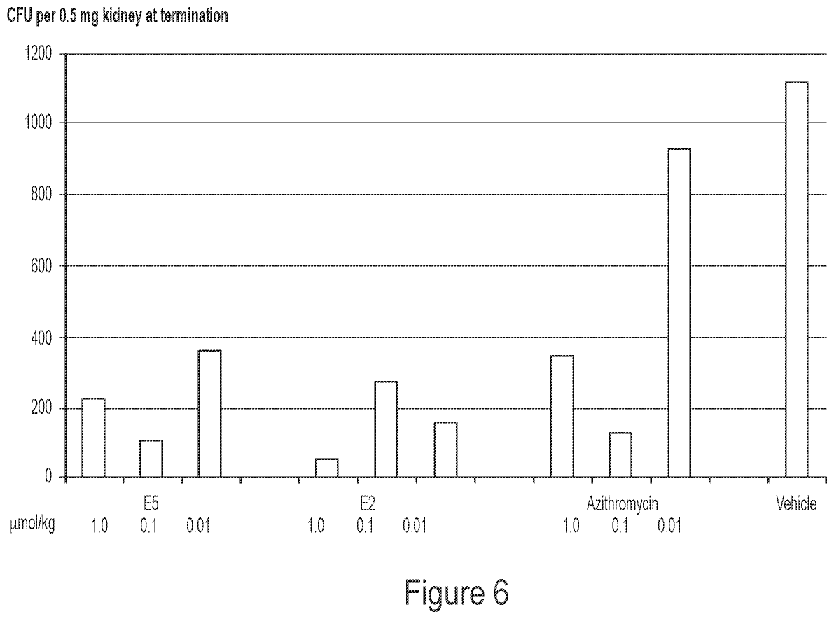

FIG. 6: Effect of substances E2, E5 and Azithromycin on the ability of mice to clear an infection by Staphylococcus aureus Newman. Bacteria are quantified as CFU recovered from a standard sample of kidney. Treatment with substances decreases recovered bacteria in a dose responsive manner.

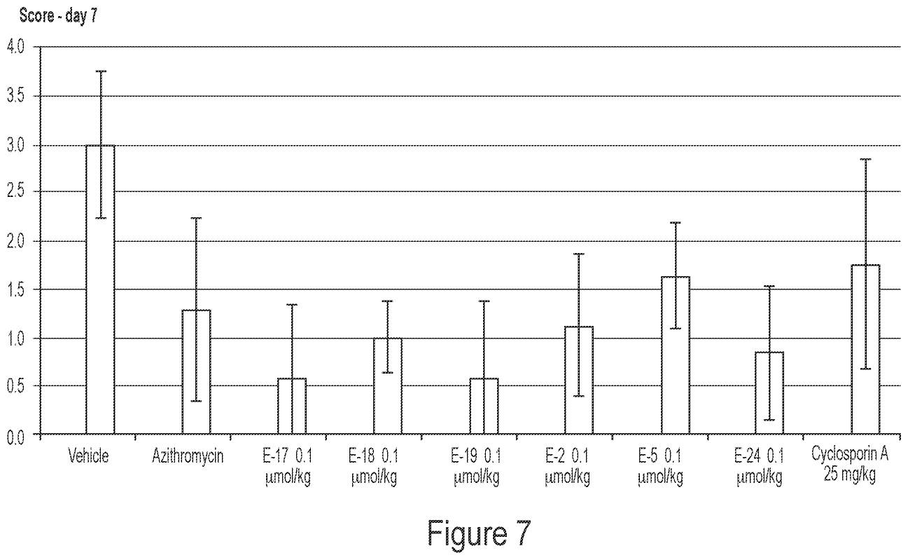

FIG. 7: Effect of substances on the ability of mice to tolerate dextran sulfate colitis. Cyclosporine is provided at a dose of 25 mg/kg, all other substances including azithromycin are provided at a dose of 0.1 μmol/kg.

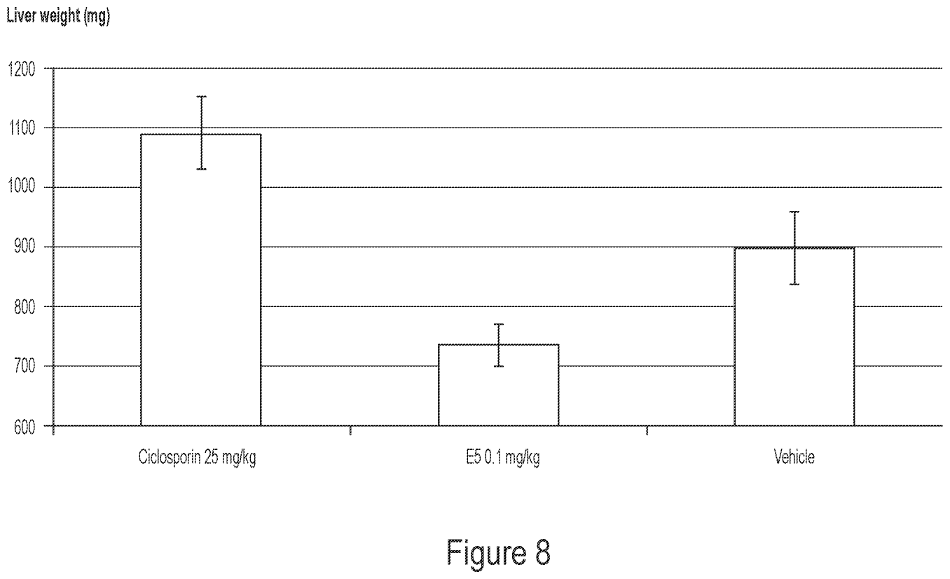

FIG. 8: Effect of substances E5 and Cyclosporin on the liver weight of mice treated with dextran sulfate colitis. Data are the mean of N=8 and are plotted with the 95% confidence interval.

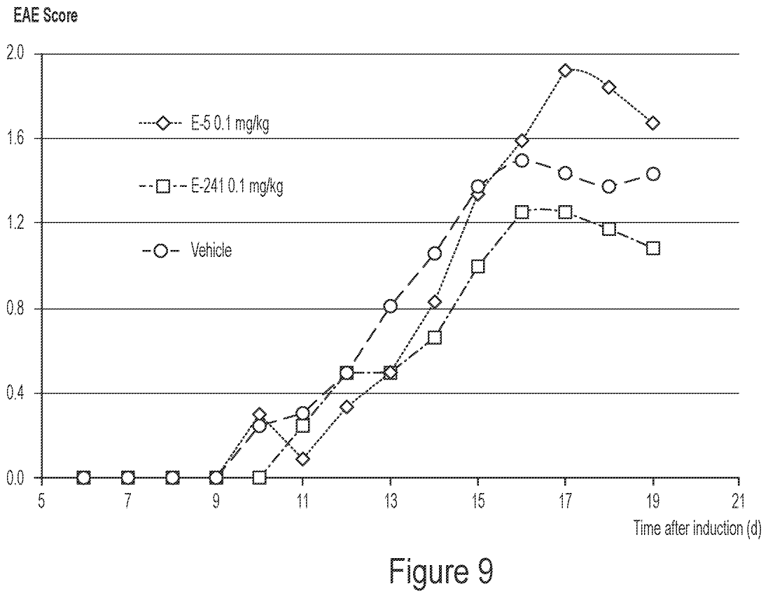

FIG. 9: Effect of substances E-5 and E-241 versus Vehicle on the development of EAE in the C57B6 mouse. Plotted is the clinical score based with antigen injected on day 0 and substance started on day 7. Data are the mean of N=8.

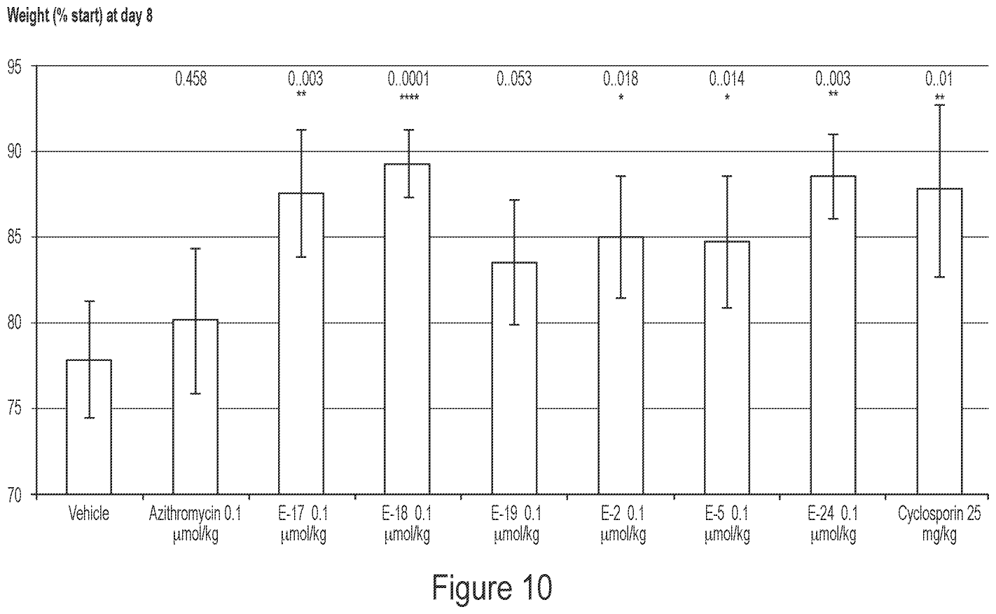

FIG. 10: Effect of substances compared with the positive control Cyclosporin on the body weight of mice treated with dextran sulfate colitis. Data are the mean of N=8 and are plotted with the 95% confidence interval. Above the bars are the p values vs. Vehicle for a T-test.

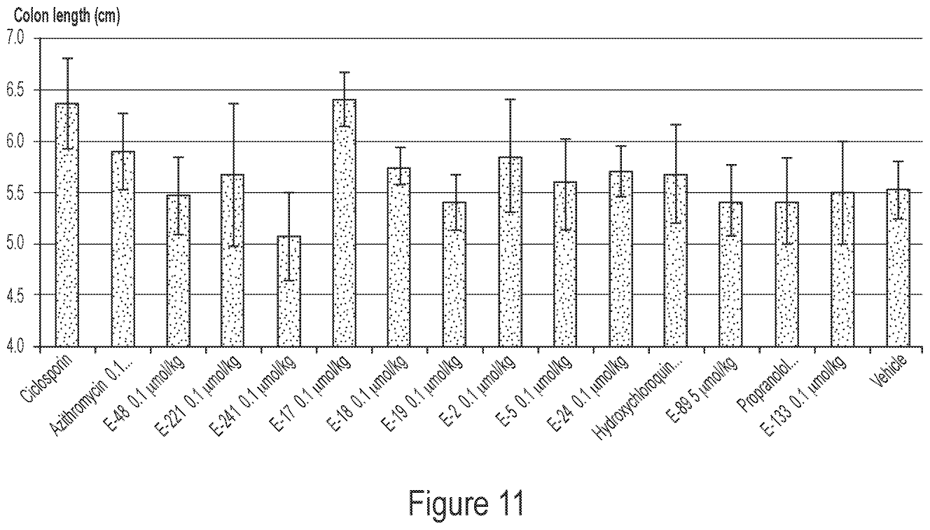

FIG. 11: Effect of substances compared with the positive control Cyclosporin on the colon length of mice treated with dextran sulfate colitis. Data are the mean of N=8 and are plotted with the 95% confidence interval. Above the bars are the p values vs. Vehicle for a T-test.

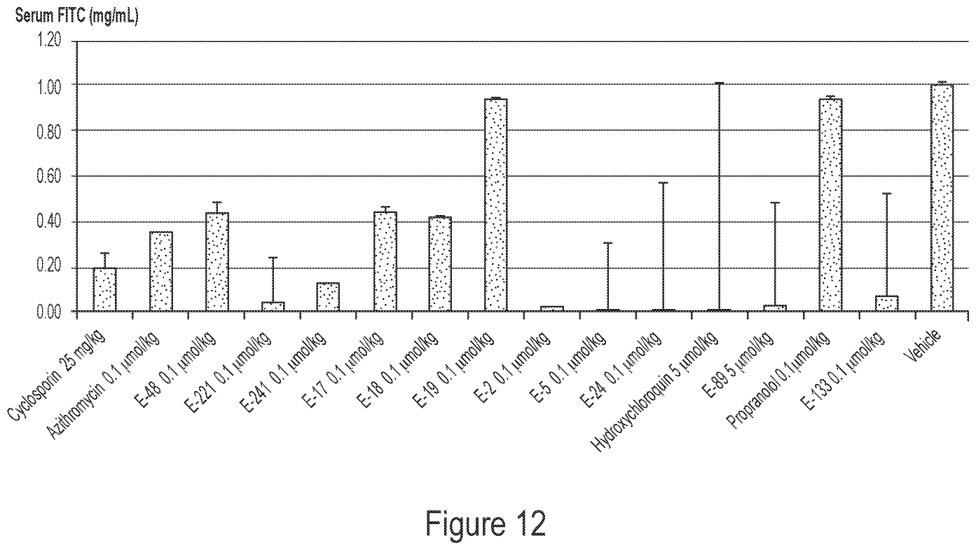

FIG. 12: Effect of substances compared with the positive control Cyclosporin on the amount of fluorescein labelled dextran (FITC) taken up into the serum of mice treated with dextran sulfate colitis. 4 h prior to sampling, mice are treated with an oral suspension of FITC dextran which would normally not enter the blood stream. The effect of DSS is to disrupt the gut epithelium allowing larger molecules to enter the blood stream. Reductions in FITC dextran suggest improved barrier function. Data are the mean of N=8 and are plotted with the 95% confidence interval. Above the bars are the p values vs. Vehicle for a T-test.

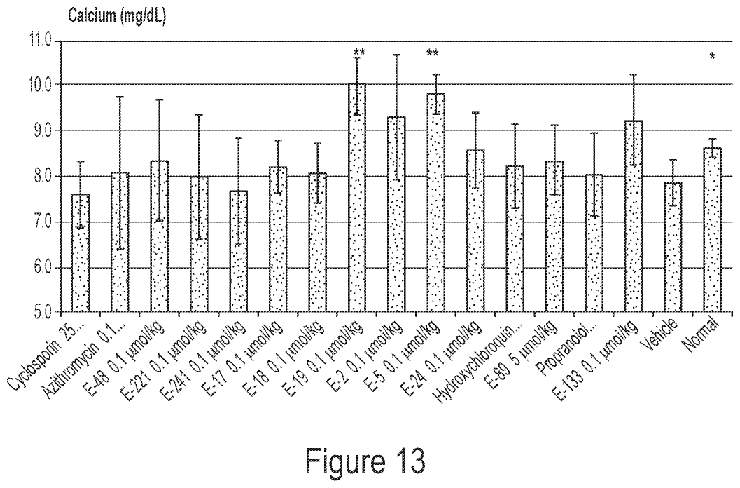

FIG. 13: Effect of substances compared with the positive control Cyclosporin on the serum Calcium of mice treated with dextran sulfate to induce colitis at day 8 after starting DSS. Data are the mean of N=8 and are plotted with the 95% confidence interval. Above the bars are the p values vs. Vehicle for a T-test.

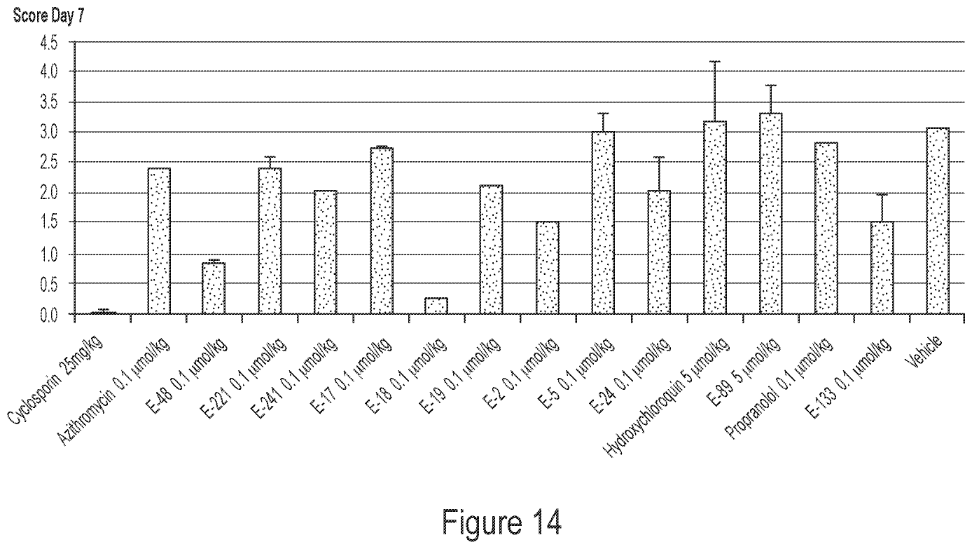

FIG. 14: Effect of substances compared with the positive control Cyclosporin on the clinical score of mice treated with dextran sulfate to induce colitis at day 8 after starting DSS. Data are the mean of N=8 and are plotted with the 95% confidence interval. Above the bars are the p values vs. Vehicle for a T-test.

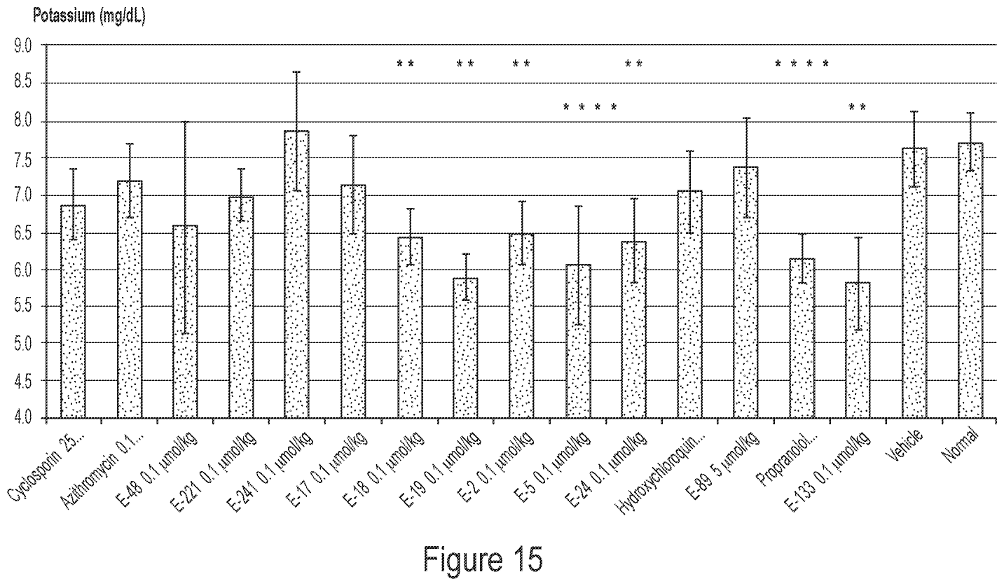

FIG. 15: Effect of substances compared with the positive control Cyclosporin on the serum Potassium of mice treated with dextran sulfate to induce colitis at day 8 after starting DSS. Data are the mean of N=8 and are plotted with the 95% confidence interval. Above the bars are the p values vs. Vehicle for a T-test.

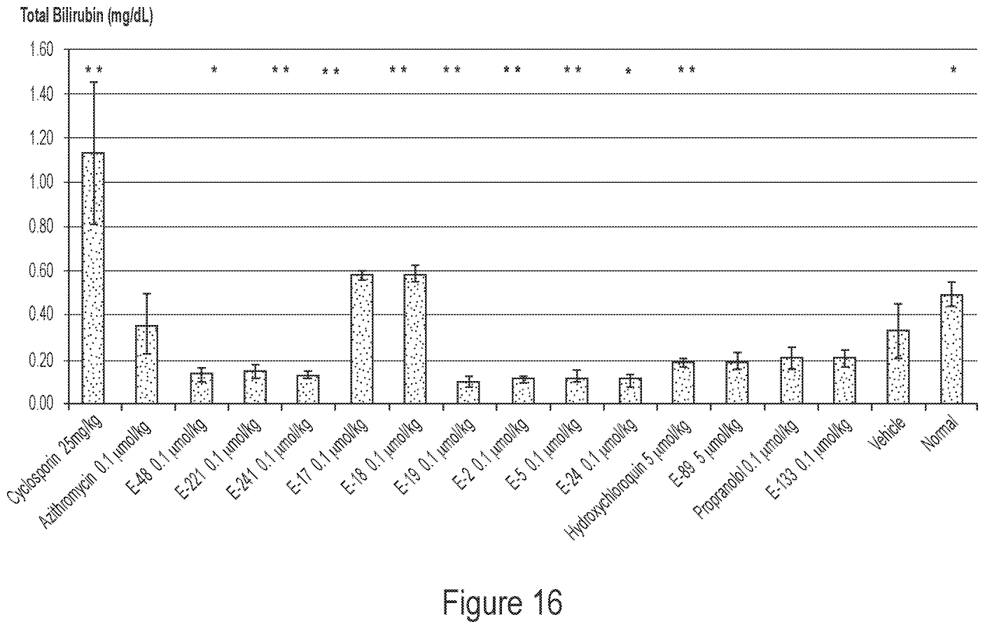

FIG. 16: Effect of substances compared with the positive control Cyclosporin on the serum Total Bilirubin of mice treated with dextran sulfate to induce colitis at day 8 after starting DSS. Data are the mean of N=8 and are plotted with the 95% confidence interval. Above the bars are the p values vs. Vehicle for a T-test.

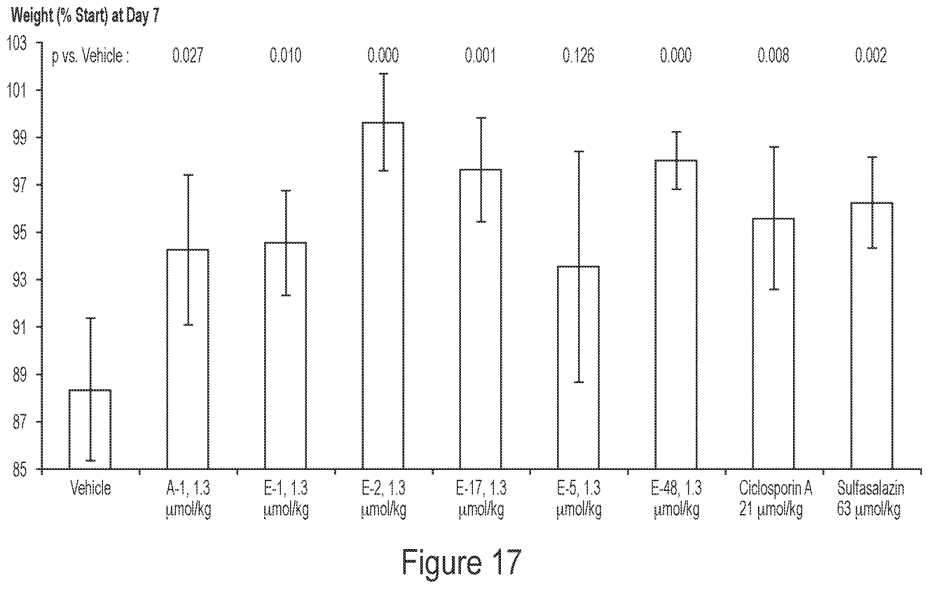

FIG. 17: Body weight of BALBc mice at day 7 after commencing 2.5% DSS in water. DSS causes lesions in the colon that lead to weight loss. Substance E-2, amongst others, protects against weight loss. Data are the mean of N=8 and are plotted with the 95% confidence interval.

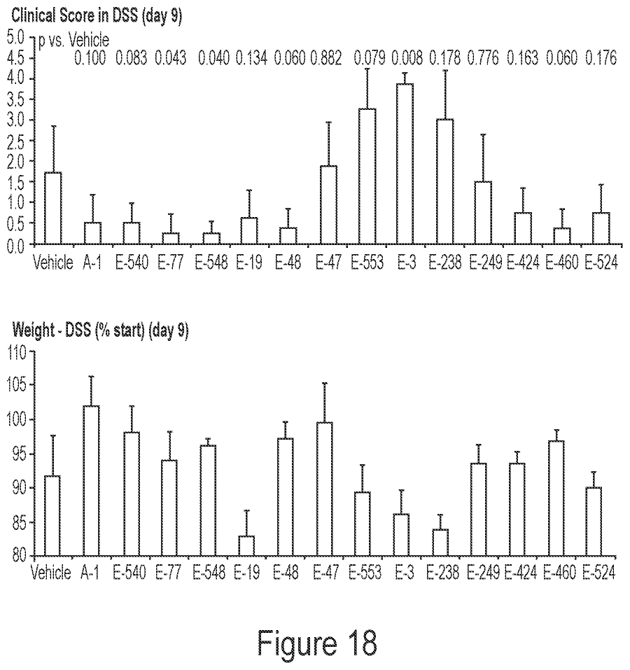

FIG. 18: Body weight and clinical score of BALBc mice at day 9 after commencing 2.5% DSS in water. DSS causes lesions in the colon that lead to weight loss. Substances E-3, E-238 and E-553, amongst others, stimulate inflammation. All doses 1.34 μmol/kg. Data are the mean of N=8 and are plotted with the 95% confidence interval. Above the bars are the p values vs. Vehicle for a T-test.

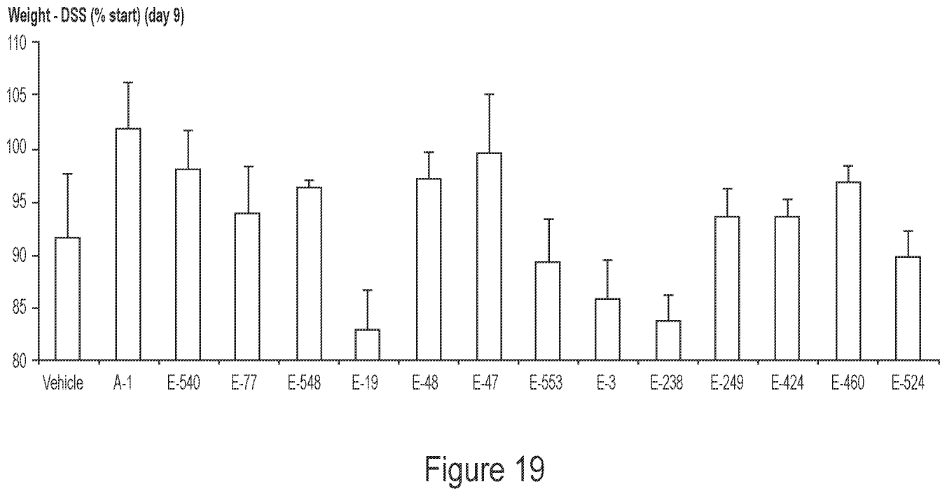

FIG. 19: Body weight and clinical score of BALBc mice at day 7 after commencing 2.5% DSS in water. DSS causes lesions in the colon that lead to weight loss. Substance like E-51 with weight greater than Vehicle protect against inflammation. Doses are as indicated. Data are the mean of N=8 and are plotted with the 95% confidence interval. Above the bars are the p values vs. Vehicle for a T-test.

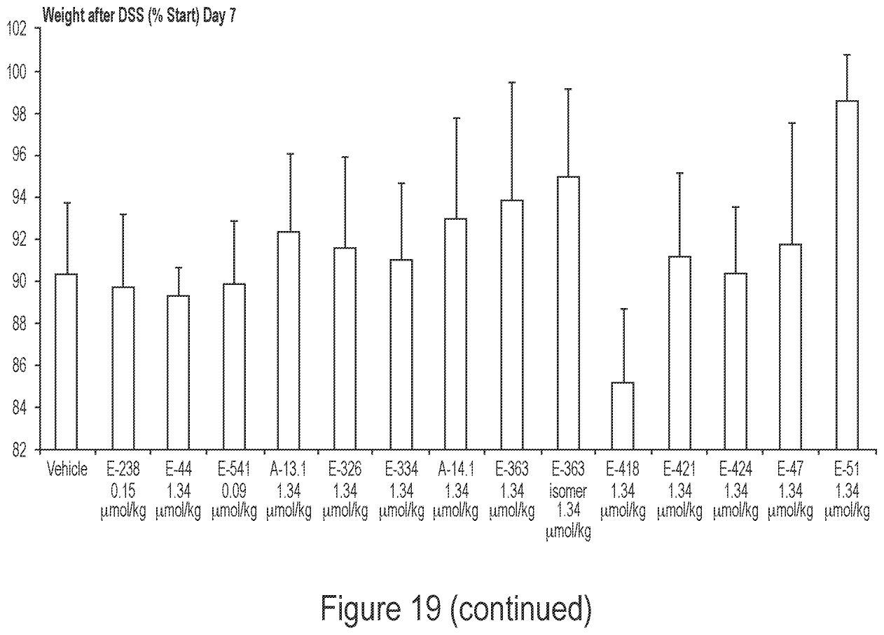

FIG. 20: The compounds containing R¹ nitrate ester have preferential distribution to the lung. Data show the concentration of the substance in the lung and liver at 6 h after administration of a 10 mg/kg dose p.o. in 2% citric acid.

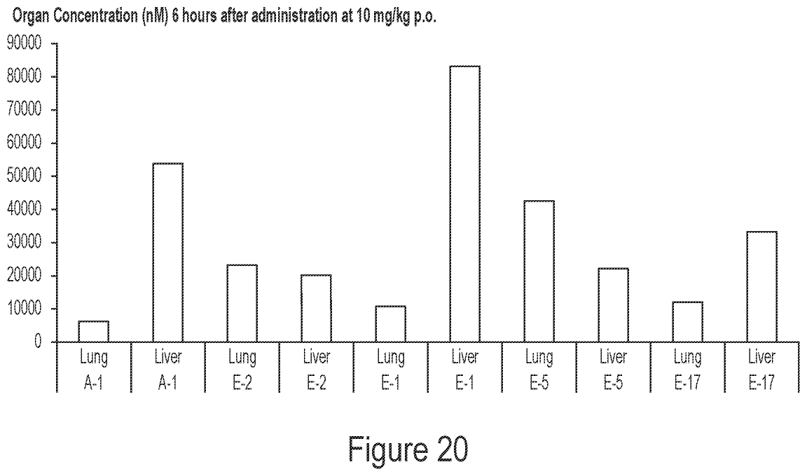

FIG. 21: The effect of various compounds on the rate of killing of Salmonella typhimurium following incubation and phagocytosis by J774 murine cells. The number of surviving bacteria is an indicator of the degree of intracellular killing of the bacteria, All substances are supplied at an initial concentration of 1 μM.

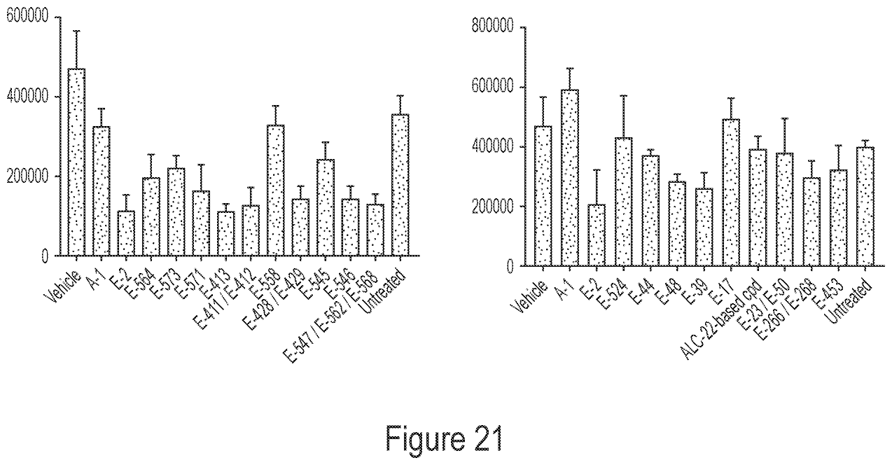

### DESCRIPTION OF EMBODIMENTS

### General Procedure for the Introduction of the Nitrooxide Group

The compound to be nitrated (1 equiv.) (—SH, —OH, —NH) is dissolved or suspended in acetic acid (approximately 6.0 ml per 1 mmol compound to be nitrated) and a solution of nitric acid (10% in acetic anhydride, about 3.25 ml per 1 mmol compound to be nitrated) is slowly added to the system while cooling in an ice bath. When TLC indicated complete consumption of starting materials the mixture is poured onto ice hydrolyzing any remains of acetic anhydride, followed by cautious neutralization of acid species with sodium bicarbonate. Extraction of the aqueous system with dichloromethane (3×), drying of combined organic phases over sodium sulfate and subsequent purification of crude products by column chromatography (acetone-cyclohexane 1:3→1:1) yields products as amorphous white foams.

The invention will be further described in the following intermediates and examples. It should be understood that these examples are for illustrative purposes only and are not to be construed as limiting this invention in any manner.

### EXAMPLES

Unless otherwise specified, all commercially available reagents and solvents were used without prior purification. All chemical structures and names are generated from ChemDraw Ultra (Cambridge).

TABLE 1
Examples of ALC Core

| Entry | ALC Core | Structure |
|---|---|---|
| A-1 | Azithromycin (R₁ to R₅ = H, R₆ = CH₃) | 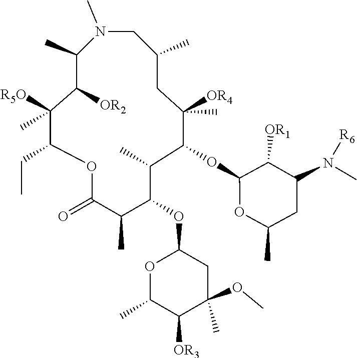 |
| A-2 | Erythromycin (X = OH) (R₁ to R₅ = H, R₆ = CH3) or Erythromycin N-Oxime (X = N—OH) | 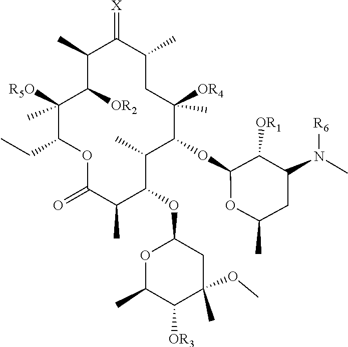 |
| A-3 | Hydroxychloroquine (R₁ = H, R₂ = CH₃) | 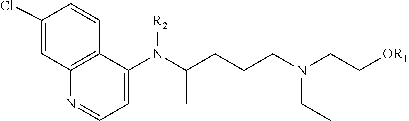 |
| A-4 | N-ethanol HCQ (R₁ = H, R₂ = H) | 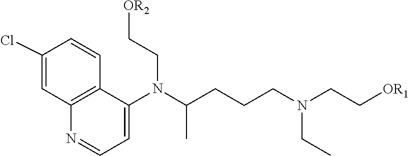 |
| A-5 | Propranolol (R₁ = H, R₂ = CH₃) | 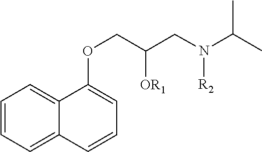 |
| A-6 | N-ethanol-propranolol (R₁ = H, R₂ = H) | 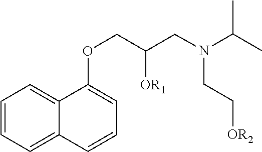 |
| A-7 | 4-hydroxypropranolol (R₁ = H, R₂ = H, R₃ = H) | 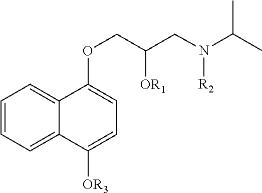 |
| A-8 | 2-(4-fluorophenyl)-3-amino-4-(3-[2,3- di{butyroyloxy}propyloxy]phenyl) carbonylpyrazole (R₁ = H, R₂ = H) | 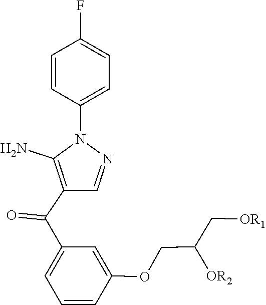 |
| A-9 | 2-(4-pyridyl)-3-amino-4-(3-[2,3- di{butyroyloxy}propyloxy]- phenyl)carbonylpyrazole (R₁ = H, R₂ = H) | 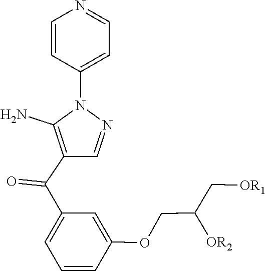 |
| A-10 | CSY0073 (R₁ to R₅ = H, R₆ = CH₃) or 14-(4-Dimethylamino-3-hydroxy-6- methyl-tetrahydro-pyran-2-yloxy)- 5-ethyl-1,6,7-trihydroxy- 2,6,8,9,ll,13,15-heptamethyl-4,16- dioxa-9-aza- bicyclo[11.2.1]hexadecan-3-one | 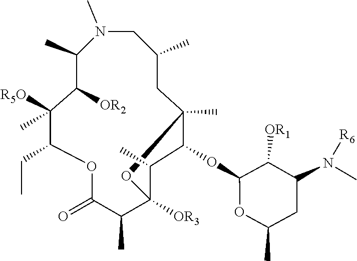 |
| A-11/E-16 | CSY0041 (R₁ to R₆ = H) or 2-Ethyl-3,4,10-trihydroxy-13-(5- hydroxy-4-methoxy-4,6-dimethyl- tetrahydro-pyran-2-yloxy)-11-(3- hydroxy-6-methyl-4-methylamino- tetrahydro-pyran-2-yloxy)- 3,5,6,8,10,12,14-heptamethyl-1- oxa-6-aza-cyclopentadecan-15-one | 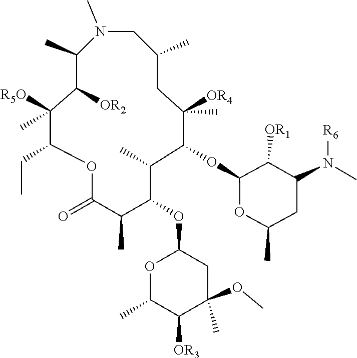 |
| A-12 | CSY1239 (R₁ to R₅ = H) or 11-(4-Dimethylamino-3-hydroxy-6- methyl-tetrahydro-pyran-2-yloxy)- 2-ethyl-3,4,10,13-tetrahydroxy-3,5, 6,8,10,12,14-heptamethyl-1-oxa-6- aza-cyclopentadecan-15-one | 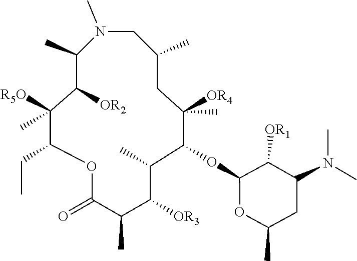 |
| A-13.1 | CSY1130 (R₁ to R₆ = H) (preparation see Example or 11-{4-[Bis-(2-hydroxy-ethyl)- amino]-3-hydroxy-6-methyl- tetrahydro-pyran-2-yloxy}-2-ethyl- 3,4,10-trihydroxy-13-(5-hydroxy-4- methoxy-4,6-dimethyl-tetrahydro- pyran-2-yloxy)-3,5,6,8,10,12,14- heptamethyl-1-oxa-6- aza-cyclopentadecan-15-one | 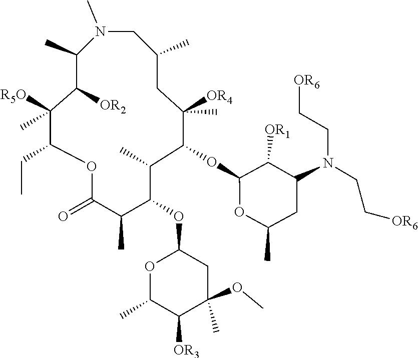 |
| A-13.2 | CSY5632 (R₁ to R₆ = H) or (2R,3S,4R,5R,8R,10R,11R,12S,13S, 14R)-11-(((2S,3S,6R)-3-(bis(2- hydroxyethyl)amino)-4-hydroxy-6- methyltetrahydro-2H-pyran-2- yl)oxy)-2-ethyl-3,4,10-trihydroxy- 13-(((2R,4R,5S,6S)-5-hydroxy-4- methoxy-4,6-dimethyltetrahydro- 2H-pyran-2-yl)oxy)- 3,5,6,8,10,12,14-heptamethyl-1- oxa-6-azacyclopentadecan-15-one | 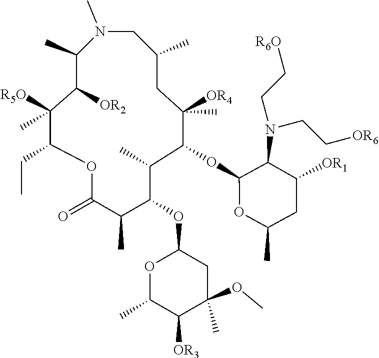 |
| A-14.1 | CSY2219 (R₁ to R₅ = H) or 2-Ethyl-3,4,10-trihydroxy-13-(5- hydroxy-4-methoxy-4,6-dimethyl- tetrahydro-pyran-2-yloxy)-11-(3- hydroxy-6-methyl-4-morpholin-4- yl-tetrahydro-pyran-2-yloxy)- 3,5,6,8,10,12,14-heptamethyl-1- oxa-6-aza-cyclopentadecan-15-one | 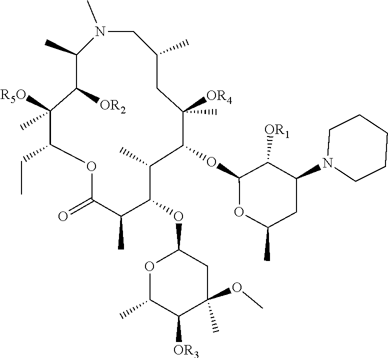 |
| A-14.2 | CSY5602 (R₁ to R₅ = H) or (2R,3S,4R,5R,8R,10R,11R,12S,13S, 14R)-2-ethyl-3,4,10-trihydroxy-13- (((2R,4R,5S,6S)-5-hydroxy-4- methoxy-4,6-dimethyltetrahydro- 2H-pyran-2-yl)oxy)-11-(((2S,3S,6R)- 4-hydroxy-6-methyl-3- morpholinotetrahydro-2H-pyran-2- yl)oxy)-3,5,6,8,10,12,14- heptamethyl-1-oxa-6- azacyclopentadecan-15-one | 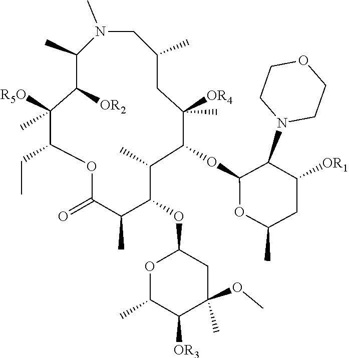 |
| A-15/E-1 | CSY5667 (R₁, R₂, R₄, R₅ = H) or (2S,3S,4R,6R)-6- (((2R,3S,4R,5R,8R,10R,11R,12S,13S, 14R)-11-(((2S,3R,4S,6R)-4- (dimethylamino)-3-hydroxy-6- methyltetrahydro-2H-pyran-2- yl)oxy)-2-ethyl-3,4,10-trihydroxy- 3,5,6,8,10,12,14-heptamethyl-15- oxo-1-oxa-6-azacyclopentadecan- 13-yl)oxy)-4-methoxy-2,4- dimethyltetrahydro-2H-pyran-3-yl nitrate | 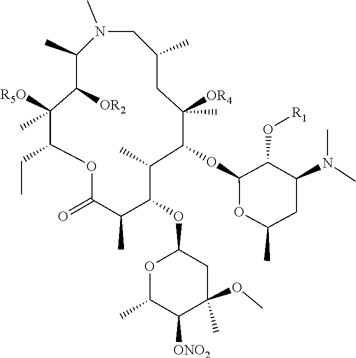 |
| A-16 | Tildipirosin (R₁ to R₃ = H) | 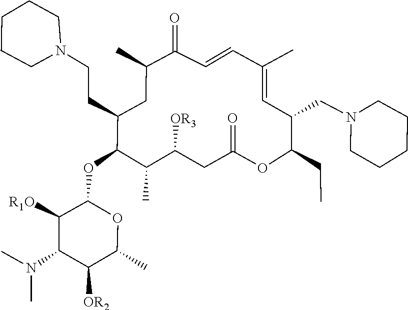 |
| A-17 | Gamithromycin (R₁ to R₅ = H) | 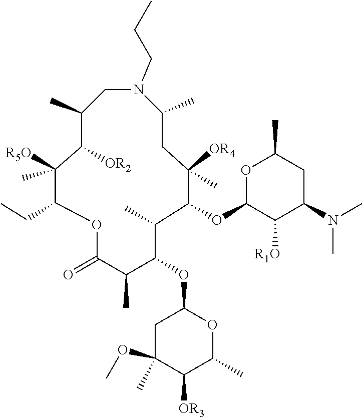 |
| A-18 | Tylosin (R₁ to R₅ = H) | 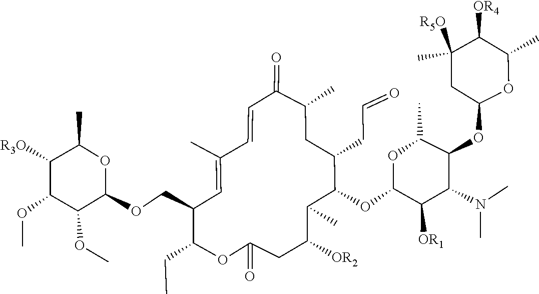 |
| A-19.1 | Polyamines (R₁ to R₂ = H) | Typical example but not limiting: 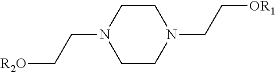 |
| A-19.2 | Polyamines (R₁ to R₄ = H) | Typical example but not limiting: 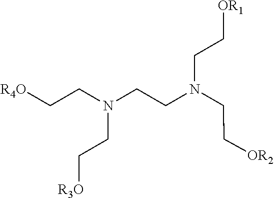 |
| A-19.3 | Polyamines (R₁ = R₂ = H, R₃ = alkyl, usually ethyl) | Typical example but not limiting: 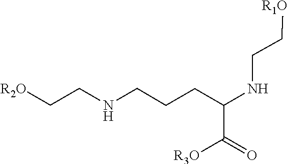 |
| A-20.1 | Tris(hydroxymethyl)nitromethane (R₁to R₃ = H) | 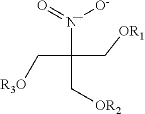 |
| A-20.2 | Sodium Tris(hydroxymethyl)aminopropylsulfonate (R₁ to R₃ = H) | 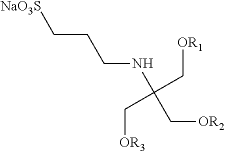 |
| A-21 | Clarithromycin (R₁ to R₄ = H) | 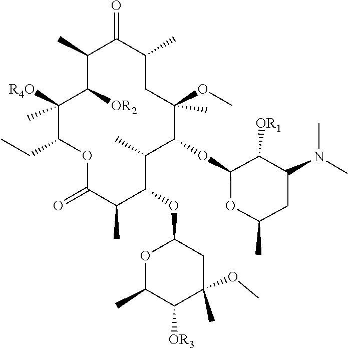 |
| A-22 | Tulathromycin (R₁ to R₅ = 0) | 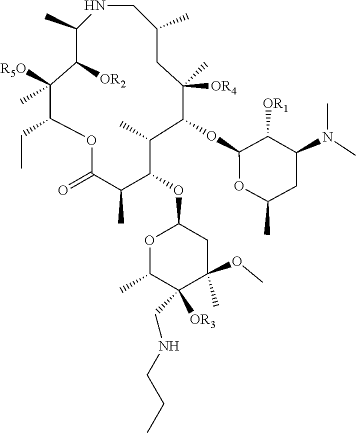 |
| A-23 | 3-descladinosyl-3- triacetylfucosylazithromycin or (3S,4R,5R,6S)-2- (((2R,3S,4R,5R,8R,10R,11R,12S,13S, 14R)-11-(((2S,3R,4S,6R)-4- (dimethylamino)-3-hydroxy-6- methyltetrahydro-2H-pyran-2- yl)oxy)-2-ethyl-3,4,10-trihydroxy- 3,5,6,8,10,12,14-heptamethyl-15- oxo-1-oxa-6-azacyclopentadecan- 13-yl)oxy)-6-methyltetrahydro-2H- pyran-3,4,5-triyltriacetate | 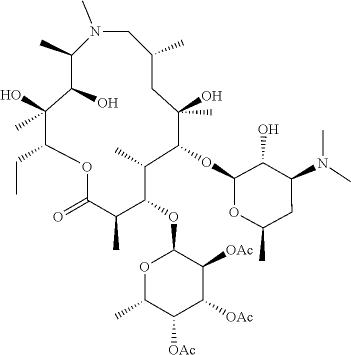 [SMILES: CC[C@H]1OC(=O)[C@H](C)[C@@H](O[C@@H]2O[C@@H](C)[C@@H](OC(C)=O)[C@@H](OC(C)=O)[C@@H]2OC(C)=O)[C@H](C)[C@@H](O[C@@H]2O[C@H](C)C[C@H](N(C)C)[C@H]2O)[C@](C)(O)C[C@@H](C)CN(C)[C@H](C)[C@@H](O)[C@]1(C)O] |
| A-24 | (2R,3S,4R,5R,8R,10R,11R,12S,13S, 14R)-2-ethyl-3,4,10-trihydroxy-11- (((2S,3R,4R,6R)-3-hydroxy-4-((2- hydroxy-3-((2- hydroxyethyl)(propyl)amino)propyl) (methyl)amino)-6- methyltetrahydro-2H-pyran-2- yl)oxy)-13-(((2R,4R,58,6S)-5- hydroxy-4-methoxy-4,6- dimethyltetrahydro-2H-pyran-2- yl)oxy)-3,5,6,8,10,12,14- heptamethyl-1-oxa-6- azacyclopentadecan-15-one | 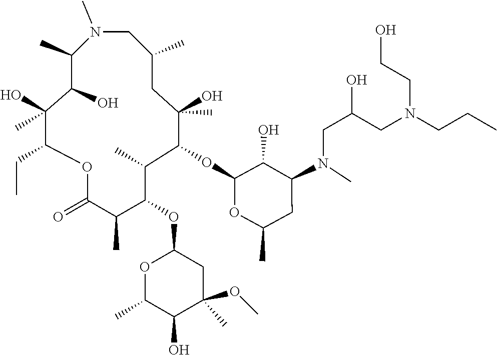 [SMILES: CCCN(CCO)CC(O)CN(C)[C@H]1C[C@@H](C)O[C@@H](O[C@@H]2[C@@H](C)[C@H](O[C@H]3C[C@@](C)(OC)[C@@H](O)[C@H](C)O3)[C@@H](C)C(=O)O[C@H](CC)[C@@](C)(O)[C@H](O)[C@@H](C)N(C)C[C@H](C)C[C@@]2(C)O)[C@@H]1O] |
| A-25 | (2R,3S,4R,5R,8R,10R,11R,12S,13S, 14R)-2-ethyl-3,4,10,13-tetrahydroxy- 11-(((2S,3R,4S,6R)-3-hydroxy-6- methyl-4- (methylamino)tetrahydro-2H- pyran-2-yl)oxy)-3,5,6,8,10,12,14- heptamethyl-1-oxa-6- azacyclopentadecan-15-one | 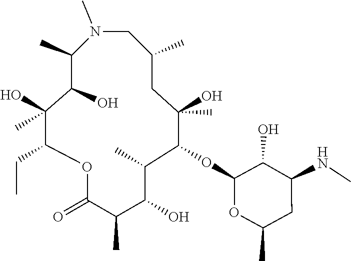 [SMILES: CC[C@H]1OC(=O)[C@H](C)[C@@H](O)[C@H](C)[C@@H](O[C@@H]2O[C@H](C)C[C@H](NC)[C@H]2O)[C@](C)(O)C[C@@H](C)CN(C)[C@H](C)[C@@H](O)[C@]1(C)O] |

TABLE 2
Examples of compounds showing the appropriate substituents.
Compound formulae based on “ALC core” structures as detailed in Table 1 (above).

| Compound | | | | | | | |
|---|---|---|---|---|---|---|---|
| Entry | ALC Core | R₁ | R₂ | R₃ | R₄ | R₅ | R₆ |
| E-1 | Azithromycin | H | H | NO₂ | H | H | CH₃ |
| E-2 | Azithromycin | NO₂ | Ac | NO₂ | H | H | CH₃ |
| E-3 | Azithromycin | NO₂ | H | NO₂ | H | H | C-10 alkyl |
| E-4 | Azithromycin | Ac | H | NO₂ | H | H | C-10 alkyl |
| E-5 | Azithromycin | Propionyl | H | NO₂ | H | H | CH₃ |
| E-6 | Azithromycin | p-Nitrobenzoyl | NO₂ | NO₂ | H | H | CH₃ |
| E-7 | Azithromycin | Benzoyl | NO₂ | H | H | H | CH₃ |
| E-8 | Erythromycin | Methoxyacetyl | H | NO₂ | H | H | CH₃ |
| | oxime | | | | | | |
| E-9 | Azithromycin | Ac | NO₂ | H | H | H | CH₃ |
| E-10 | Azithromycin | H | NO₂ | H | H | H | CH₃ |
| E-11 | Azithromycin | NO₂ | CH₃ | H | H | H | CH₃ |
| E-12 | Azithromycin | H | Tetranitro | H | H | H | CH₃ |
| | | | moiety¹ | | | | |
| E-13 | Azithromycin | Propionyl | Propionyl | NO₂ | H | Propionyl | CH₃ |
| E-14 | Azithromycin | H | H | H | H | H | Propanol- |
| | | | | | | | NO₂ |
| E-15 | Azithromycin | H | H | H | H | H | CH₃ |
| E-16 | Azithromycin | H | H | H | H | H | H |
| E-17/I-7 | Azithromycin | H | Ac | H | H | H | CH₃ |
| E-18 | Azithromycin | H | Propionyl | H | H | H | CH₃ |
| E-19 | Azithromycin | H | Butyryl | H | H | H | CH₃ |
| E-20/I-2 | Azithromycin | H | H | H | H | H | C-10 alkyl |
| E-21 | Azithromycin | NO₂ | NO₂ | H | H | H | CH₃ |
| E-22/I-4 | Azithromycin | Ac | CH₂CCH | H | H | H | CH₃ |
| E-23/I-6 | Azithromycin | Ac | Ac | H | H | H | CH₃ |
| E-24 | Azithromycin | Butyryl | H | NO₂ | H | H | CH₃ |
| E-25//I-3 | Azithromycin | Ac | H | H | H | H | CH₃ |
| E-26/I-8 | Azithromycin | Benzoyl | H | H | H | H | CH₃ |
| E-27 | Azithromycin | Succinyl | H | H | H | H | CH₃ |
| E-28 | Azithromycin | Ac | Propionyl | H | H | H | CH₃ |
| E-29 | Azithromycin | Ac | Butyryl | H | H | H | CH₃ |
| E-30 | Azithromycin | Propionyl | H | H | H | H | CH₃ |
| E-31 | Azithromycin | Propionyl | NO₂ | H | H | H | CH₃ |
| E-32 | Azithromycin | Propionyl | Ac | H | H | H | CH₃ |
| E-33 | Azithromycin | Propionyl | Propionyl | H | H | H | CH₃ |
| E-34 | Azithromycin | Propionyl | Butyryl | H | H | H | CH₃ |
| E-35 | Azithromycin | Butyryl | H | H | H | H | CH₃ |
| E-36 | Azithromycin | Butyryl | NO₂ | H | H | H | CH₃ |
| E-37 | Azithromycin | Butyryl | Ac | H | H | H | CH₃ |
| E-38 | Azithromycin | Butyryl | Propionyl | H | H | H | CH₃ |
| E-39 | Azithromycin | Butyryl | Butyryl | H | H | H | CH₃ |
| E-40 | Azithromycin | H | Succinyl | H | H | H | CH₃ |
| E-41 | Azithromycin | H | Pyruvyl | H | H | H | CH₃ |
| E-42 | Azithromycin | H | Maleyl | H | H | H | CH₃ |
| E-43 | Azithromycin | H | Lactyl | H | H | H | CH₃ |
| E-44 | Azithromycin | H | Isobutyryl | H | H | H | CH₃ |
| E-45 | Azithromycin | H | Valeryl | H | H | H | CH₃ |
| E-46 | Azithromycin | H | Isovaleryl | H | H | H | CH₃ |
| E-47 | Azithromycin | Butyryl | Butyryl | Butyryl | Butyryl | H | CH₃ |
| E-48 | Azithromycin | Butyryl | Butyryl | Butyryl | H | H | CH₃ |
| E-49 | Azithromycin | Ac | Ac | Ac | Ac | H | CH₃ |
| E-50 | Azithromycin | Ac | Ac | Ac | H | H | CH₃ |
| E-51 | Azithromycin | Propionyl | Propionyl | Propionyl | Propionyl | H | CH₃ |
| E-52 | Azithromycin | Propionyl | Propionyl | Propionyl | H | H | CH₃ |
| E-53 | Azithromycin | Succinyl | Succinyl | Succinyl | Succinyl | H | CH₃ |
| E-54 | Azithromycin | Pyruvyl | Pyruvyl | Pyruvyl | Pyruvyl | H | CH₃ |
| E-55 | Azithromycin | Maleyl | Maleyl | Maleyl | Maleyl | H | CH₃ |
| E-56 | Azithromycin | Lactyl | Lactyl | Lactyl | Lactyl | H | CH₃ |
| E-57 | Azithromycin | Isobutyryl | Isobutyryl | Isobutyryl | Isobutyryl | H | CH₃ |
| E-58 | Azithromycin | Valeryl | Valeryl | Valeryl | Valeryl | H | CH₃ |
| E-59 | Azithromycin | Isovaleryl | Isovaleryl | Isovaleryl | Isovaleryl | H | CH₃ |
| E-60 | Azithromycin | Succinyl | Succinyl | Succinyl | H | H | CH₃ |
| E-61 | Azithromycin | Pyruvyl | Pyruvyl | Pyruvyl | H | H | CH₃ |
| E-62 | Azithromycin | Maleyl | Maleyl | Maleyl | H | H | CH₃ |
| E-63 | Azithromycin | Lactyl | Lactyl | Lactyl | H | H | CH₃ |
| E-64 | Azithromycin | Isobutyryl | Isobutyryl | Isobutyryl | H | H | CH₃ |
| E-65 | Azithromycin | Valeryl | Valeryl | Valeryl | H | H | CH₃ |
| E-66 | Azithromycin | Isovaleryl | Isovaleryl | Isovaleryl | H | H | CH₃ |
| E-67 | Azithromycin | Succinyl | Succinyl | H | H | H | CH₃ |
| E-68 | Azithromycin | Pyruvyl | Pyruvyl | H | H | H | CH₃ |
| E-69 | Azithromycin | Maleyl | Maleyl | H | H | H | CH₃ |
| E-70 | Azithromycin | Lactyl | Lactyl | H | H | H | CH₃ |
| E-71 | Azithromycin | Isobutyryl | Isobutyryl | H | H | H | CH₃ |
| E-72 | Azithromycin | Valeryl | Valeryl | H | H | H | CH₃ |
| E-73 | Azithromycin | Isovaleryl | Isovaleryl | H | H | H | CH₃ |
| E-74 | Azithromycin | Acetoxypropionyl | H | H | H | H | CH₃ |
| E-75 | Azithromycin | Lipoyl | Lipoyl | H | H | H | CH₃ |
| E-76 | Azithromycin | H | Lipoyl | H | H | H | CH₃ |
| E-77 | Azithromycin | Lipoyl | H | H | H | H | CH₃ |
| E-78 | Azithromycin | Lipoyl | H | NO₂ | H | H | CH₃ |
| E-79 | Azithromycin | Succinyl - | H | NO₂ | H | H | CH₃ |
| | | dithiole-3-thione | | | | | |
| E-80 | Azithromycin | Succinyl - | H | H | H | H | CH₃ |
| | | dithiole-3-thione | | | | | |
| E-81 | Azithromycin | Polysulfide | H | H | H | H | CH₃ |
| | | ethyl carbonate | | | | | |
| E-82/ | Azithromycin | NO₂ | H | H | H | H | CH₃ |
| CSY1019 | | | | | | | |
| E-83 | Azithromycin | O-Phenyl | H | H | H | H | CH₃ |
| | | chlorothionocarbonate | | | | | |
| E-84 | Azithromycin | n-hexanoyl | H | H | H | H | CH₃ |
| E-85 | Azithromycin | Bromoethylcarbonate | H | H | H | H | CH₃ |
| E-86 | Azithromycin | Vinyl | H | H | H | H | CH₃ |
| | | carbonate | | | | | |
| E-86-d | Azithromycin | H | Propionyl | Propionyl | H | H | CH₃ |
| E-86-e | Azithromycin | Propionyl | H | Propionyl | H | H | CH₃ |
| E-86-f | Azithromycin | Propionyl | H | H | H | H | CH₃ |
| E-86-g | Azithromycin | Valeroyl | H | NO₂ | H | H | CH₃ |
| E-86-h | Azithromycin | Butyroyl | H | Butyroyl | H | H | CH₃ |
| E-86-i | Azithromycin | H | H | Propionyl | H | H | CH₃ |
| E-87 | Hydroxychloroquine | Ac | H | | | | |
| E-88 | Hydroxychloroquine | Propionyl | H | | | | |
| E-89 | Hydroxychloroquine | Butyryl | H | | | | |
| E-90 | Hydroxychloroquine | Succinyl | H | | | | |
| E-91 | Hydroxychloroquine | Pyruvyl | H | | | | |
| E-92 | Hydroxychloroquine | Maleyl | H | | | | |
| E-93 | Hydroxychloroquine | Lactyl | H | | | | |
| E-94 | Hydroxychloroquine | Isobutyryl | H | | | | |
| E-95 | Hydroxychloroquine | Valeryl | H | | | | |
| E-96 | Hydroxychloroquine | Isovaleryl | H | | | | |
| E-97 | Hydroxychloroquine | Lipoyl | H | | | | |
| E-98 | Hydroxychloroquine | Ac | CH₃ | | | | |
| E-99 | Hydroxychloroquine | Propionyl | CH₃ | | | | |
| E-100 | Hydroxychloroquine | Butyryl | CH₃ | | | | |
| E-101 | Hydroxychloroquine | Succinyl | CH₃ | | | | |
| E-102 | Hydroxychloroquine | Pyruvyl | CH₃ | | | | |
| E-103 | Hydroxychloroquine | Maleyl | CH₃ | | | | |
| E-104 | Hydroxychloroquine | Lactyl | CH₃ | | | | |
| E-105 | Hydroxychloroquine | Isobutyryl | CH₃ | | | | |
| E-106 | Hydroxychloroquine | Valeryl | CH₃ | | | | |
| E-107 | Hydroxychloroquine | Isovaleryl | CH₃ | | | | |
| E-108 | Hydroxychloroquine | Lipoyl | CH₃ | | | | |
| E-109 | N-ethanol HCQ | NO₂ | Ac | | | | |
| E-110 | N-ethanol HCQ | NO₂ | Propionyl | | | | |
| E-111 | N-ethanol HCQ | NO₂ | Butyryl | | | | |
| E-112 | N-ethanol HCQ | NO₂ | Succinyl | | | | |
| E-113 | N-ethanol HCQ | NO₂ | Pyruvyl | | | | |
| E-114 | N-ethanol HCQ | NO₂ | Maleyl | | | | |
| E-115 | N-ethanol HCQ | NO₂ | Lactyl | | | | |
| E-116 | N-ethanol HCQ | NO₂ | Isobutyryl | | | | |
| E-117 | N-ethanol HCQ | NO₂ | Valeryl | | | | |
| E-118 | N-ethanol HCQ | NO₂ | Isovaleryl | | | | |
| E-119 | N-ethanol HCQ | NO₂ | Lipoyl | | | | |
| E-120 | N-ethanol HCQ | Ac | NO₂ | | | | |
| E-121 | N-ethanol HCQ | Propionyl | NO₂ | | | | |
| E-122 | N-ethanol HCQ | Butyryl | NO₂ | | | | |
| E-123 | N-ethanol HCQ | Succinyl | NO₂ | | | | |
| E-124 | N-ethanol HCQ | Pyruvyl | NO₂ | | | | |
| E-125 | N-ethanol HCQ | Maleyl | NO₂ | | | | |
| E-126 | N-ethanol HCQ | Lactyl | NO₂ | | | | |
| E-127 | N-ethanol HCQ | Isobutyryl | NO₂ | | | | |
| E-128 | N-ethanol HCQ | Valeryl | NO₂ | | | | |
| E-129 | N-ethanol HCQ | Isovaleryl | NO₂ | | | | |
| E-130 | N-ethanol HCQ | Lipoyl | NO₂ | | | | |
| E-131 | Propranolol | Ac | H | | | | |
| E-132 | Propranolol | Propionyl | H | | | | |
| E-133 | Propranolol | Butyryl | H | | | | |
| E-134 | Propranolol | Succinyl | H | | | | |
| E-135 | Propranolol | Pyruvyl | H | | | | |
| E-136 | Propranolol | Maleyl | H | | | | |
| E-137 | Propranolol | Lactyl | H | | | | |
| E-138 | Propranolol | Isobutyryl | H | | | | |
| E-139 | Propranolol | Valeryl | H | | | | |
| E-140 | Propranolol | Isovaleryl | H | | | | |
| E-141 | Propranolol | Lipoyl | H | | | | |
| E-142 | Propranolol | 2-O-Nitrolactyl | H | | | | |
| E-143 | Propranolol | Ac | CH₃ | | | | |
| E-144 | Propranolol | Propionyl | CH₃ | | | | |
| E-145 | Propranolol | Butyryl | CH₃ | | | | |
| E-146 | Propranolol | Succinyl | CH₃ | | | | |
| E-147 | Propranolol | Pyruvyl | CH₃ | | | | |
| E-148 | Propranolol | Maleyl | CH₃ | | | | |
| E-149 | Propranolol | Lactyl | CH₃ | | | | |
| E-150 | Propranolol | Isobutyryl | CH₃ | | | | |
| E-151 | Propranolol | Valeryl | CH₃ | | | | |
| E-152 | Propranolol | Isovaleryl | CH₃ | | | | |
| E-153 | Propranolol | Lipoyl | CH₃ | | | | |
| E-154 | N-ethanol- | NO₂ | Ac | | | | |
| | propranolol | | | | | | |
| E-155 | N-ethanol- | NO₂ | Propionyl | | | | |
| | propranolol | | | | | | |
| E-156 | N-ethanol- | NO₂ | Butyryl | | | | |
| | propranolol | | | | | | |
| E-157 | N-ethanol- | NO₂ | Succinyl | | | | |
| | propranolol | | | | | | |
| E-158 | N-ethanol- | NO₂ | Pyruvyl | | | | |
| | propranolol | | | | | | |
| E-159 | N-ethanol- | NO₂ | Maleyl | | | | |
| | propranolol | | | | | | |
| E-160 | N-ethanol- | NO₂ | Lactyl | | | | |
| | propranolol | | | | | | |
| E-161 | N-ethanol- | NO₂ | Isobutyryl | | | | |
| | propranolol | | | | | | |
| E-162 | N-ethanol- | NO₂ | Valeryl | | | | |
| | propranolol | | | | | | |
| E-163 | N-ethanol- | NO₂ | Isovaleryl | | | | |
| | propranolol | | | | | | |
| E-164 | N-ethanol- | NO₂ | Lipoyl | | | | |
| | propranolol | | | | | | |
| E-165 | N-ethanol- | Propionyl | NO₂ | | | | |
| | propranolol | | | | | | |
| E-166 | N-ethanol- | Butyryl | NO₂ | | | | |
| | propranolol | | | | | | |
| E-167 | N-ethanol- | Succinyl | NO₂ | | | | |
| | propranolol | | | | | | |
| E-168 | N-ethanol- | Pyruvyl | NO₂ | | | | |
| | propranolol | | | | | | |
| E-169 | N-ethanol- | Maleyl | NO₂ | | | | |
| | propranolol | | | | | | |
| E-170 | N-ethanol- | Lactyl | NO₂ | | | | |
| | propranolol | | | | | | |
| E-171 | N-ethanol- | Isobutyryl | NO₂ | | | | |
| | propranolol | | | | | | |
| E-172 | N-ethanol- | Valeryl | NO₂ | | | | |
| | propranolol | | | | | | |
| E-173 | N-ethanol- | Isovaleryl | NO₂ | | | | |
| | propranolol | | | | | | |
| E-174 | N-ethanol- | Lipoyl | NO₂ | | | | |
| | propranolol | | | | | | |
| E-175 | 4-hydroxy | NO₂ | H | Ac | | | |
| | propranolol | | | | | | |
| E-176 | 4-hydroxy | NO₂ | H | Propionyl | | | |
| | propranolol | | | | | | |
| E-177 | 4-hydroxy | NO₂ | H | Butyryl | | | |
| | propranolol | | | | | | |
| E-178 | 4-hydroxy | NO₂ | H | Succinyl | | | |
| | propranolol | | | | | | |
| E-179 | 4-hydroxy | NO₂ | H | Pyruvyl | | | |
| | propranolol | | | | | | |
| E-180 | 4-hydroxy | NO₂ | H | Maleyl | | | |
| | propranolol | | | | | | |
| E-181 | 4-hydroxy | NO₂ | H | Lactyl | | | |
| | propranolol | | | | | | |
| E-182 | 4-hydroxy | NO₂ | H | Isobutyryl | | | |
| | propranolol | | | | | | |
| E-183 | 4-hydroxy | NO₂ | H | Valeryl | | | |
| | propranolol | | | | | | |
| E-184 | 4-hydroxy | NO₂ | H | Isovaleryl | | | |
| | propranolol | | | | | | |
| E-185 | 4-hydroxy | NO₂ | H | Lipoyl | | | |
| | propranolol | | | | | | |
| E-186 | 4-hydroxy | NO₂ | CH₃ | Ac | | | |
| | propranolol | | | | | | |
| E-187 | 4-hydroxy | NO₂ | CH₃ | Propionyl | | | |
| | propranolol | | | | | | |
| E-188 | 4-hydroxy | NO₂ | CH₃ | Butyryl | | | |
| | propranolol | | | | | | |
| E-189 | 4-hydroxy | NO₂ | CH₃ | Succinyl | | | |
| | propranolol | | | | | | |
| E-190 | 4-hydroxy | NO₂ | CH₃ | Pyruvyl | | | |
| | propranolol | | | | | | |
| E-191 | 4-hydroxy | NO₂ | CH₃ | Maleyl | | | |
| | propranolol | | | | | | |
| E-192 | 4-hydroxy | NO₂ | CH₃ | Lactyl | | | |
| | propranolol | | | | | | |
| E-193 | 4-hydroxy | NO₂ | CH₃ | Isobutyryl | | | |
| | propranolol | | | | | | |
| E-194 | 4-hydroxy | NO₂ | CH₃ | Valeryl | | | |
| | propranolol | | | | | | |
| E-195 | 4-hydroxy | NO₂ | CH₃ | Isovaleryl | | | |
| | propranolol | | | | | | |
| E-196 | 4-hydroxy | NO₂ | CH₃ | Lipoyl | | | |
| | propranolol | | | | | | |
| E-197 | A-8 | Ac | Ac | | | | |
| E-198 | A-8 | Propionyl | Propionyl | | | | |
| E-199 | A-8 | Butyryl | Butyryl | | | | |
| E-200 | A-8 | Succinyl | Succinyl | | | | |
| E-201 | A-8 | Pyruvyl | Pyruvyl | | | | |
| E-202 | A-8 | Maleyl | Maleyl | | | | |
| E-203 | A-8 | Lactyl | Lactyl | | | | |
| E-204 | A-8 | Isobutyryl | Isobutyryl | | | | |
| E-205 | A-8 | Valeryl | Valeryl | | | | |
| E-206 | A-8 | Isovaleryl | Isovaleryl | | | | |
| E-207 | A-8 | Lipoyl | Lipoyl | | | | |
| E-208 | A-9 | Ac | Ac | | | | |
| E-209 | A-9 | Propionyl | Propionyl | | | | |
| E-210 | A-9 | Butyryl | Butyryl | | | | |
| E-211 | A-9 | Succinyl | Succinyl | | | | |
| E-212 | A-9 | Pyruvyl | Pyruvyl | | | | |
| E-213 | A-9 | Maleyl | Maleyl | | | | |
| E-214 | A-9 | Lactyl | Lactyl | | | | |
| E-215 | A-9 | Isobutyryl | Isobutyryl | | | | |
| E-216 | A-9 | Valeryl | Valeryl | | | | |
| E-217 | A-9 | Isovaleryl | Isovaleryl | | | | |
| E-218 | A-9 | Lipoyl | Lipoyl | | | | |
| E-219 | A-10 | H | Tetranitro | H | H | H | CH₃ |
| | | | moiety1 | | | | |
| E-220 | A-10 | H | H | H | H | H | Propanol- |
| | | | | | | | NO2 |
| E-221 | A-10 | H | H | H | H | H | CH₃ |
| E-222 | A-10 | H | H | H | H | H | H |
| E-223 | A-10 | H | Ac | H | H | H | CH₃ |
| E-224 | A-10 | H | Propionyl | H | H | H | CH₃ |
| E-225 | A-10 | H | Butyryl | H | H | H | CH₃ |
| E-226 | A-10 | H | H | H | H | H | C-10 alkyl |
| E-227 | A-10 | Ac | CH₂CCH | H | H | H | CH₃ |
| E-228 | A-10 | Ac | Ac | H | H | H | CH₃ |
| E-229 | A-10 | Ac | H | H | H | H | CH₃ |
| E-230 | A-10 | Benzoyl | H | H | H | H | CH₃ |
| E-231 | A-10 | Succinyl | H | H | H | H | CH₃ |
| E-232 | A-10 | Ac | Propionyl | H | H | H | CH₃ |
| E-233 | A-10 | Ac | Butyryl | H | H | H | CH₃ |
| E-234 | A-10 | Propionyl | H | H | H | H | CH₃ |
| E-235 | A-10 | Propionyl | Ac | H | H | H | CH₃ |
| E-236 | A-10 | Propionyl | Propionyl | H | H | H | CH₃ |
| E-237 | A-10 | Propionyl | Butyryl | H | H | H | CH₃ |
| E-238 | A-10 | Butyryl | H | H | H | H | CH₃ |
| E-239 | A-10 | Butyryl | Ac | H | H | H | CH₃ |
| E-240 | A-10 | Butyryl | Propionyl | H | H | H | CH₃ |
| E-241 | A-10 | Butyryl | Butyryl | H | H | H | CH₃ |
| E-242 | A-10 | H | Succinyl | H | H | H | CH₃ |
| E-243 | A-10 | H | Pyruvyl | H | H | H | CH₃ |
| E-244 | A-10 | H | Maleyl | H | H | H | CH₃ |
| E-245 | A-10 | H | Lactyl | H | H | H | CH₃ |
| E-246 | A-10 | H | Isobutyryl | H | H | H | CH₃ |
| E-247 | A-10 | H | Valeryl | H | H | H | CH₃ |
| E-248 | A-10 | H | Isovaleryl | H | H | H | CH₃ |
| E-249 | A-10 | Butyryl | Butyryl | H | Butyryl | H | CH₃ |
| E-250 | A-10 | Butyryl | Butyryl | H | H | H | CH₃ |
| E-251 | A-10 | Ac | Ac | H | Ac | H | CH₃ |
| E-252 | A-10 | Ac | Ac | H | H | H | CH₃ |
| E-253 | A-10 | Propionyl | Propionyl | H | Propionyl | H | CH₃ |
| E-254 | A-10 | Propionyl | Propionyl | H | H | H | CH₃ |
| E-255 | A-11/E-16 | Butyric | H | H | H | H | Butyric |
| E-256 | A-11/E-16 | Butyric | Butyric | H | H | H | Butyric |
| E-257 | A-11/E-16 | Butyric | Butyric | Butyric | H | H | Butyric |
| E-258 | A-11/E-16 | H | H | H | H | H | Mannose |
| E-259 | A-11/E-16 | Propionic | H | H | H | H | Propionic |
| E-260 | A-11/E-16 | Propionic | Propionic | H | H | H | Propionic |
| E-261 | A-11/E-16 | Propionic | Propionic | Propionic | H | H | Propionic |
| E-262 | A-11/E-16 | Ac | H | H | H | H | Ac |
| E-263 | A-11/E-16 | Ac | Ac | H | H | H | Ac |
| E-264 | A-11/E-16 | Ac | Ac | Ac | H | H | Ac |
| E-265 | A-12 | NO₂ | H | H | H | H | CH₃ |
| E-266 | A-12 | Ac | Ac | H | H | H | CH₃ |
| E-267 | A-12 | Ac | Ac | Ac | H | H | CH₃ |
| E-268 | A-12 | Ac | Ac | Ac | Ac | H | CH₃ |
| E-269 | A-12 | Propionic | Propionic | H | H | H | CH₃ |
| E-270 | A-12 | Propionic | Propionic | Propionic | H | H | CH₃ |
| E-271 | A-12 | Propionic | Propionic | Propionic | Propionic | H | CH₃ |
| E-272 | A-12 | Butyric | Butyric | H | H | H | CH₃ |
| E-273 | A-12 | Butyric | Butyric | Butyric | H | H | CH₃ |
| E-274 | A-12 | Butyric | Butyric | Butyric | Butyric | H | CH₃ |
| E-275 | A-12 | Succinic | Succinic | H | H | H | CH₃ |
| E-276 | A-12 | Pyruvic | Pyruvic | H | H | H | CH₃ |
| E-277 | A-12 | Maleic | Maleic | H | H | H | CH₃ |
| E-278 | A-12 | Lactic | Lactic | H | H | H | CH₃ |
| E-279 | A-12 | Isobutyric | Isobutyric | H | H | H | CH₃ |
| E-280 | A-12 | Isobutyric | Isobutyric | Isobutyric | H | H | CH₃ |
| E-281 | A-12 | Isobutyric | Isobutyric | Isobutyric | Isobutyric | H | CH₃ |
| E-282 | A-12 | Valeric | Valeric | H | H | H | CH₃ |
| E-283 | A-12 | Valeric | Valeric | Valeric | H | H | CH₃ |
| E-284 | A-12 | Valeric | Valeric | Valeric | Valeric | H | CH₃ |
| E-285 | A-12 | Isovaleric | Isovaleric | H | H | H | CH₃ |
| E-286 | A-12 | Isovaleric | Isovaleric | Isovaleric | H | H | CH₃ |
| E-287 | A-12 | Isovaleric | Isovaleric | Isovaleric | Isovaleric | H | CH₃ |
| E-288 | A-12 | Lipoic | Lipoic | H | H | H | CH₃ |
| E-289 | A-12 | Lipoic | Lipoic | Lipoic | H | H | CH₃ |
| E-290 | A-12 | Lipoic | Lipoic | Lipoic | Lipoic | H | CH₃ |
| E-291 | A-13.1 | H | H | H | H | H | Ac |
| E-292 | A-13.1 | Ac | H | H | H | H | Ac |
| E-293 | A-13.1 | Ac | Ac | H | H | H | Ac |
| E-294 | A-13.1 | Ac | Ac | Ac | H | H | Ac |
| E-295 | A-13.1 | H | H | H | H | H | Propionic |
| E-296 | A-13.1 | Propionic | H | H | H | H | Propionic |
| E-297 | A-13.1 | Propionic | Propionic | H | H | H | Propionic |
| E-298 | A-13.1 | Propionic | Propionic | Propionic | H | H | Propionic |
| E-298-a | A-13.1 | Propionic | H | NO₂ | H | H | Propionic |
| E-299 | A-13.1 | H | H | H | H | H | Butyric |
| E-300 | A-13.1 | Butyric | H | H | H | H | Butyric |
| E-301 | A-13.1 | Butyric | Butyric | H | H | H | Butyric |
| E-302 | A-13.1 | Butyric | Butyric | Butyric | H | H | Butyric |
| E-303 | A-13.1 | H | H | H | H | H | Succinic |
| E-304 | A-13.1 | H | H | H | H | H | Pyruvic |
| E-305 | A-13.1 | H | H | H | H | H | Maleic |
| E-306 | A-13.1 | H | H | H | H | H | Lactic |
| E-307 | A-13.1 | H | H | H | H | H | Isobutyric |
| E-308 | A-13.1 | Isobutyric | H | H | H | H | Isobutyric |
| E-309 | A-13.1 | Isobutyric | Isobutyric | H | H | H | Isobutyric |
| E-310 | A-13.1 | Isobutyric | Isobutyric | Isobutyric | H | H | Isobutyric |
| E-311 | A-13.1 | H | H | H | H | H | Valeric |
| E-312 | A-13.1 | Valeric | H | H | H | H | Valeric |
| E-313 | A-13.1 | Valeric | Valeric | H | H | H | Valeric |
| E-314 | A-13.1 | Valeric | Valeric | Valeric | H | H | Valeric |
| E-315 | A-13.1 | H | H | H | H | H | Isovaleric |
| E-316 | A-13.1 | Isovaleric | H | H | H | H | Isovaleric |
| E-317 | A-13.1 | Isovaleric | Isovaleric | H | H | H | Isovaleric |
| E-318 | A-13.1 | Isovaleric | Isovaleric | Isovaleric | H | H | Isovaleric |
| E-319 | A-13.1 | H | H | H | H | H | Lipoic |
| E-320 | A-13.1 | Lipoic | H | H | H | H | Lipoic |
| E-321 | A-13.1 | Lipoic | Lipoic | H | H | H | Lipoic |
| E-322 | A-13.1 | Lipoic | Lipoic | Lipoic | H | H | Lipoic |
| E-322-c | A-13.1 | H | H | H | H | H | NO₂ |
| E-323 | A-13.2 | H | H | H | H | H | Ac |
| E-324 | A-13.2 | Ac | H | H | H | H | Ac |
| E-325 | A-13.2 | Ac | Ac | H | H | H | Ac |
| E-326 | A-13.2 | Ac | Ac | Ac | H | H | Ac |
| E-327 | A-13.2 | H | H | H | H | H | Propionic |
| E-328 | A-13.2 | Propionic | H | H | H | H | Propionic |
| E-329 | A-13.2 | Propionic | Propionic | H | H | H | Propionic |
| E-330 | A-13.2 | Propionic | Propionic | Propionic | H | H | Propionic |
| E-330-a | A-13.2 | Propionic | H | NO₂ | H | H | Propionic |
| E-331 | A-13.2 | H | H | H | H | H | Butyric |
| E-332 | A-13.2 | Butyric | H | H | H | H | Butyric |
| E-333 | A-13.2 | Butyric | Butyric | H | H | H | Butyric |
| E-334 | A-13.2 | Butyric | Butyric | Butyric | H | H | Butyric |
| E-335 | A-13.2 | H | H | H | H | H | Succinic |
| E-336 | A-13.2 | H | H | H | H | H | Pyruvic |
| E-337 | A-13.2 | H | H | H | H | H | Maleic |
| E-338 | A-13.2 | H | H | H | H | H | Lactic |
| E-339 | A-13.2 | H | H | H | H | H | Isobutyric |
| E-340 | A-13.2 | Isobutyric | H | H | H | H | Isobutyric |
| E-341 | A-13.2 | Isobutyric | Isobutyric | H | H | H | Isobutyric |
| E-342 | A-13.2 | Isobutyric | Isobutyric | Isobutyric | H | H | Isobutyric |
| E-343 | A-13.2 | H | H | H | H | H | Valeric |
| E-344 | A-13.2 | Valeric | H | H | H | H | Valeric |
| E-345 | A-13.2 | Valeric | Valeric | H | H | H | Valeric |
| E-346 | A-13.2 | Valeric | Valeric | Valeric | H | H | Valeric |
| E-347 | A-13.2 | H | H | H | H | H | Isovaleric |
| E-348 | A-13.2 | Isovaleric | H | H | H | H | Isovaleric |
| E-349 | A-13.2 | Isovaleric | Isovaleric | H | H | H | Isovaleric |
| E-350 | A-13.2 | Isovaleric | Isovaleric | Isovaleric | H | H | Isovaleric |
| E-351 | A-13.2 | H | H | H | H | H | Lipoic |
| E-352 | A-13.2 | Lipoic | H | H | H | H | Lipoic |
| E-353 | A-13.2 | Lipoic | Lipoic | H | H | H | Lipoic |
| E-354 | A-13.2 | Lipoic | Lipoic | Lipoic | H | H | Lipoic |
| E-354-c | A-13.2 | H | H | H | H | H | NO₂ |
| E-355 | A-14.1 | Ac | H | H | H | H | |
| E-356 | A-14.1 | Ac | Ac | H | H | H | |
| E-357 | A-14.1 | Ac | Ac | Ac | H | H | |
| E-358 | A-14.1 | Propionic | H | H | H | H | |
| E-359 | A-14.1 | Propionic | Propionic | H | H | H | |
| E-359-a | A-14.1 | Propionic | H | Propionic | H | H | |
| E-360 | A-14.1 | Propionic | Propionic | Propionic | H | H | |
| E-360-a | A-14.1 | Propionic | H | NO₂ | H | H | |
| E-360-b | A-14.1 | Propionic | Propionic | NO₂ | H | H | |
| E-361 | A-14.1 | Butyric | H | H | H | H | |
| E-362 | A-14.1 | Butyric | Butyric | H | H | H | |
| E-363 | A-14.1 | Butyric | Butyric | Butyric | H | H | |
| E-364 | A-14.1 | Succinic | H | H | H | H | |
| E-365 | A-14.1 | Pyruvic | H | H | H | H | |
| E-366 | A-14.1 | Maleic | H | H | H | H | |
| E-367 | A-14.1 | Lactic | H | H | H | H | |
| E-368 | A-14.1 | Isobutyric | H | H | H | H | |
| E-369 | A-14.1 | Isobutyric | Isobutyric | H | H | H | |
| E-370 | A-14.1 | Isobutyric | Isobutyric | Isobutyric | H | H | |
| E-371 | A-14.1 | Valeric | H | H | H | H | |
| E-372 | A-14.1 | Valeric | Valeric | H | H | H | |
| E-373 | A-14.1 | Valeric | Valeric | Valeric | H | H | |
| E-374 | A-14.1 | Isovaleric | H | H | H | H | |
| E-375 | A-14.1 | Isovaleric | Isovaleric | H | H | H | |
| E-376 | A-14.1 | Isovaleric | Isovaleric | Isovaleric | H | H | |
| E-377 | A-14.1 | Lipoic | H | H | H | H | |
| E-378 | A-14.1 | Lipoic | Lipoic | H | H | H | |
| E-379 | A-14.1 | Lipoic | Lipoic | Lipoic | H | H | |
| E-380 | A-14.2 | Ac | H | H | H | H | |
| E-381 | A-14.2 | Ac | Ac | H | H | H | |
| E-382 | A-14.2 | Ac | Ac | Ac | H | H | |
| E-383 | A-14.2 | Propionic | H | H | H | H | |
| E-384 | A-14.2 | Propionic | Propionic | H | H | H | |
| E-384-a | A-14.2 | Propionic | H | Propionic | H | H | |
| E-385 | A-14.2 | Propionic | Propionic | Propionic | H | H | |
| E-385-a | A-14.2 | Propionic | H | NO₂ | H | H | |
| E-385-b | A-14.2 | Propionic | Pripionic | NO₂ | H | H | |
| E-386 | A-14.2 | Butyric | H | H | H | H | |
| E-387 | A-14.2 | Butyric | Butyric | H | H | H | |
| E-388 | A-14.2 | Butyric | Butyric | Butyric | H | H | |
| E-389 | A-14.2 | Succinic | H | H | H | H | |
| E-390 | A-14.2 | Pyruvic | H | H | H | H | |
| E-391 | A-14.2 | Maleic | H | H | H | H | |
| E-392 | A-14.2 | Lactic | H | H | H | H | |
| E-393 | A-14.2 | Isobutyric | H | H | H | H | |
| E-394 | A-14.2 | Isobutyric | Isobutyric | H | H | H | |
| E-395 | A-14.2 | Isobutyric | Isobutyric | Isobutyric | H | H | |
| E-396 | A-14.2 | Valeric | H | H | H | H | |
| E-397 | A-14.2 | Valeric | Valeric | H | H | H | |
| E-398 | A-14.2 | Valeric | Valeric | Valeric | H | H | |
| E-399 | A-14.2 | Isovaleric | H | H | H | H | |
| E-400 | A-14.2 | Isovaleric | Isovaleric | H | H | H | |
| E-401 | A-14.2 | Isovaleric | Isovaleric | Isovaleric | H | H | |
| E-402 | A-14.2 | Lipoic | H | H | H | H | |
| E-403 | A-14.2 | Lipoic | Lipoic | H | H | H | |
| E-404 | A-14.2 | Lipoic | Lipoic | Lipoic | H | H | |
| E-404-d | A-14.2 | H | H | NO₂ | H | H | |
| E-405 | A-15 | | Succinic | H | H | H | |
| E-406 | A-15 | | Pyruvic | H | H | H | |
| E-407 | A-15 | | Maleic | H | H | H | |
| E-408 | A-15 | | Lactic | H | H | H | |
| E-409 | A-15 | | Isobutyric | H | H | H | |
| E-410 | A-15 | | Isobutyric | Isobutyric | H | H | |
| E-411 | A-15 | | Valeric | H | H | H | |
| E-412 | A-15 | | Valeric | Valeric | H | H | |
| E-413 | A-15 | | Isovaleric | H | H | H | |
| E-414 | A-15 | | Isovaleric | Isovaleric | H | H | |
| E-415 | A-15 | | Lipoic | Lipoic | H | H | |
| E-416 | A-16 | Ac | H | H | | | |
| E-417 | A-16 | Ac | Ac | H | | | |
| E-418 | A-16 | Ac | Ac | Ac | | | |
| E-419 | A-16 | Propionic | H | H | | | |
| E-420 | A-16 | Propionic | Propionic | H | | | |
| E-421 | A-16 | Propionic | Propionic | Propionic | | | |
| E-422 | A-16 | Butyric | H | H | | | |
| E-423 | A-16 | Butyric | Butyric | H | | | |
| E-424 | A-16 | Butyric | Butyric | Butyric | | | |
| E-425 | A-16 | Isobutyric | H | H | | | |
| E-426 | A-16 | Isobutyric | Isobutyric | H | | | |
| E-427 | A-16 | Isobutyric | Isobutyric | Isobutyric | | | |
| E-428 | A-16 | Valeric | H | H | | | |
| E-429 | A-16 | Valeric | Valeric | H | | | |
| E-430 | A-16 | Valeric | Valeric | Valeric | | | |
| E-431 | A-16 | Isovaleric | H | H | | | |
| E-432 | A-16 | Isovaleric | Isovaleric | H | | | |
| E-433 | A-16 | Isovaleric | Isovaleric | Isovaleric | | | |
| E-434 | A-16 | Lipoic | H | H | | | |
| E-435 | A-16 | Lipoic | Lipoic | H | | | |
| E-436 | A-16 | Lipoic | Lipoic | Lipoic | | | |
| E-437 | A-16 | Hexanoic | H | H | | | |
| E-438 | A-16 | Hexanoic | Hexanoic | H | | | |
| E-439 | A-16 | Hexanoic | Hexanoic | Hexanoic | | | |
| E-440 | A-16 | Heptanoic | H | H | | | |
| E-441 | A-16 | Heptanoic | Heptanoic | H | | | |
| E-442 | A-16 | Heptanoic | Heptanoic | Heptanoic | | | |
| E-443 | A-16 | Octanoic | H | H | | | |
| E-444 | A-16 | Octanoic | Octanoic | H | | | |
| E-445 | A-16 | Octanoic | Octanoic | Octanoic | | | |
| E-446 | A-16 | Decanoic | H | H | | | |
| E-447 | A-16 | Decanoic | Decanoic | H | | | |
| E-448 | A-16 | Decanoic | Decanoic | Decanoic | | | |
| E-449 | A-16 | Dodecanoic | H | H | | | |
| E-450 | A-16 | Dodecanoic | Dodecanoic | H | | | |
| E-451 | A-16 | Dodecanoic | Dodecanoic | Dodecanoic | | | |
| E-451-a | A-16 | Propionic | H | NO₂ | | | |
| E-451-b | A-16 | H | Propionic | NO₂ | | | |
| E-451-c | A-16 | Propionic | Propionic | NO₂ | | | |
| E-451-d | A-16 | H | H | NO₂ | | | |
| E-452 | A-17 | Ac | H | H | H | H | |
| E-453 | A-17 | Ac | Ac | H | H | H | |
| E-454 | A-17 | Ac | Ac | Ac | H | H | |
| E-455 | A-17 | Propionic | H | H | H | H | |
| E-456 | A-17 | Propionic | Propionic | H | H | H | |
| E-457 | A-17 | Propionic | Propionic | Propionic | H | H | |
| E-458 | A-17 | Butyric | H | H | H | H | |
| E-459 | A-17 | Butyric | Butyric | H | H | H | |
| E-460 | A-17 | Butyric | Butyric | Butyric | H | H | |
| E-461 | A-17 | Isobutyric | H | H | H | H | |
| E-462 | A-17 | Isobutyric | Isobutyric | H | H | H | |
| E-463 | A-17 | Isobutyric | Isobutyric | Isobutyric | H | H | |
| E-464 | A-17 | Valeric | H | H | H | H | |
| E-465 | A-17 | Valeric | Valeric | H | H | H | |
| E-466 | A-17 | Valeric | Valeric | Valeric | H | H | |
| E-467 | A-17 | Isovaleric | H | H | H | H | |
| E-468 | A-17 | Isovaleric | Isovaleric | H | H | H | |
| E-469 | A-17 | Isovaleric | Isovaleric | Isovaleric | H | H | |
| E-470 | A-17 | Lipoic | H | H | H | H | |
| E-471 | A-17 | Lipoic | Lipoic | H | H | H | |
| E-472 | A-17 | Lipoic | Lipoic | Lipoic | H | H | |
| E-473 | A-17 | Hexanoic | H | H | H | H | |
| E-474 | A-17 | Hexanoic | Hexanoic | H | H | H | |
| E-475 | A-17 | Hexanoic | Hexanoic | Hexanoic | H | H | |
| E-476 | A-17 | Heptanoic | H | H | H | H | |
| E-477 | A-17 | Heptanoic | Heptanoic | H | H | H | |
| E-478 | A-17 | Heptanoic | Heptanoic | Heptanoic | H | H | |
| E-479 | A-17 | Octanoic | H | H | H | H | |
| E-480 | A-17 | Octanoic | Octanoic | H | H | H | |
| E-481 | A-17 | Octanoic | Octanoic | Octanoic | H | H | |
| E-482 | A-17 | Decanoic | H | H | H | H | |
| E-483 | A-17 | Decanoic | Decanoic | H | H | H | |
| E-484 | A-17 | Decanoic | Decanoic | Decanoic | H | H | |
| E-485 | A-17 | Dodecanoic | H | H | H | H | |
| E-486 | A-17 | Dodecanoic | Dodecanoic | H | H | H | |
| E-487 | A-17 | Dodecanoic | Dodecanoic | Dodecanoic | H | H | |
| E-488 | A-18 | Ac | H | H | H | H | |
| E-489 | A-18 | Ac | Ac | H | H | H | |
| E-490 | A-18 | Ac | Ac | Ac | H | H | |
| E-491 | A-18 | Propionic | H | H | H | H | |
| E-492 | A-18 | Propionic | Propionic | H | H | H | |
| E-493 | A-18 | Propionic | Propionic | Propionic | H | H | |
| E-494 | A-18 | Butyric | H | H | H | H | |
| E-495 | A-18 | Butyric | Butyric | H | H | H | |
| E-496 | A-18 | Butyric | Butyric | Butyric | H | H | |
| E-497 | A-18 | Isobutyric | H | H | H | H | |
| E-498 | A-18 | Isobutyric | Isobutyric | H | H | H | |
| E-499 | A-18 | Isobutyric | Isobutyric | Isobutyric | H | H | |
| E-500 | A-18 | Valeric | H | H | H | H | |
| E-501 | A-18 | Valeric | Valeric | H | H | H | |
| E-502 | A-18 | Valeric | Valeric | Valeric | H | H | |
| E-503 | A-18 | Isovaleric | H | H | H | H | |
| E-504 | A-18 | Isovaleric | Isovaleric | H | H | H | |
| E-505 | A-18 | Isovaleric | Isovaleric | Isovaleric | H | H | |
| E-506 | A-18 | Lipoic | H | H | H | H | |
| E-507 | A-18 | Lipoic | Lipoic | H | H | H | |
| E-508 | A-18 | Lipoic | Lipoic | Lipoic | H | H | |
| E-509 | A-18 | Hexanoic | H | H | H | H | |
| E-510 | A-18 | Hexanoic | Hexanoic | H | H | H | |
| E-511 | A-18 | Hexanoic | Hexanoic | Hexanoic | H | H | |
| E-512 | A-18 | Heptanoic | H | H | H | H | |
| E-513 | A-18 | Heptanoic | Heptanoic | H | H | H | |
| E-514 | A-18 | Heptanoic | Heptanoic | Heptanoic | H | H | |
| E-515 | A-18 | Octanoic | H | H | H | H | |
| E-516 | A-18 | Octanoic | Octanoic | H | H | H | |
| E-517 | A-18 | Octanoic | Octanoic | Octanoic | H | H | |
| E-518 | A-18 | Decanoic | H | H | H | H | |
| E-519 | A-18 | Decanoic | Decanoic | H | H | H | |
| E-520 | A-18 | Decanoic | Decanoic | Decanoic | H | H | |
| E-521 | A-18 | Dodecanoic | H | H | H | H | |
| E-522 | A-18 | Dodecanoic | Dodecanoic | H | H | H | |
| E-523 | A-18 | Dodecanoic | Dodecanoic | Dodecanoic | H | H | |
| E-524 | A-19.1 | Ac | Ac | | | | |
| E-525 | A-19.1 | Propionic | Propionic | | | | |
| E-526 | A-19.1 | Butyric | Butyric | | | | |
| E-527 | A-19.1 | Isobutyric | Isobutyric | | | | |
| E-528 | A-19.1 | Valeric | Valeric | | | | |
| E-529 | A-19.1 | Isovaleric | Isovaleric | | | | |
| E-530 | A-19.1 | Adamantylcarboxyl | Adamantylcarboxyl | | | | |
| E-531 | A-19.2 | Ac | Ac | Ac | Ac | | |
| E-532 | A-19.2 | Propionic | Propionic | Propionic | Propionic | | |
| E-533 | A-19.2 | Butyric | Butyric | Butyric | Butyric | | |
| E-534 | A-19.2 | Isobutyric | Isobutyric | Isobutyric | Isobutyric | | |
| E-535 | A-19.2 | Valeric | Valeric | Valeric | Valeric | | |
| E-536 | A-19.2 | Isovaleric | Isovaleric | Isovaleric | Isovaleric | | |
| E-537 | A-20.1 | Butyric | Butyric | Butyric | | | |
| E-538 | A-20.1 | Ac | Ac | Ac | | | |
| E-539 | A-20.1 | Propionic | Propionic | Propionic | | | |
| E-540 | A-1 | N-Phenyl | H | H | H | H | CH3 |
| | | chlorothionoformate | | | | | |
| E-541 | A-1 | Imiquimod- | H | H | H | H | CH3 |
| | | Succinate | | | | | |
| E-542 | A-1 | Resiquimod- | H | H | H | H | CH3 |
| | | Succinate | | | | | |
| E-543 | A-1 | Succinate- | H | H | H | H | CH3 |
| | | ethyl ester | | | | | |
| E-544 | A-1 | Indole-3-propionic | H | H | H | H | CH3 |
| E-545 | A-1 | Cyclopropanecarboxylic | H | H | H | H | CH3 |
| E-546 | A-1 | Cyclobutanecarboxylic | H | H | H | H | CH3 |
| E-547 | A-1 | Nicotinic | H | H | H | H | CH3 |
| E-548 | A-1 | Chenodeoxycholic | H | H | H | H | CH3 |
| E-549 | A-1 | Ferrocenylacetic | H | H | H | H | CH3 |
| E-550 | A-1 | Lipoic-S | H | H | H | H | CH3 |
| | | derivatives | | | | | |
| E-551 | A-1 | Methoxyacetic | H | H | H | H | CH3 |
| E-552 | C2 (see Table 16) | Butyric | Butyric | | | | |
| E-553 | A-1 | H | Butyric | Butyric | H | H | CH3 |
| E-554 | A-1 | H | Ac | Ac | H | H | CH3 |
| E-555 | A-1 | H | Propionic | Propionic | H | H | CH3 |
| E-556 | A-1 | O-Acetyl Lactic | | H | H | H | CH3 |
| E-557 | A-2 (X = O) | Isovaleric | H | H | H | H | CH3 |
| E-558 | A-2 (X = O) | Valeric | H | H | H | H | CH3 |
| E-559 | A-1 | Methoxyacetic | Methoxyacetic | Methoxyacetic | Methoxyacetic | H | CH3 |
| E-560 | A-1 | Cyclobutanecarboxylic | Cyclobutanecarboxylic | H | H | H | CH3 |
| E-561 | A-1 | Cyclobutanecarboxylic | Cyclobutanecarboxylic | Cyclobutanecarboxylic | H | H | CH3 |
| E-562 | A-1 | Nicotinic | Nicotinic | H | H | H | CH3 |
| E-563 | A-1 | Nicotinic | Nicotinic | Nicotinic | H | H | CH3 |
| E-564 | A-2 (X = O) | Ac | H | H | H | H | CH3 |
| E-566 | A-10 | Isobutyric | Isobutyric | Isobutyric | | H | CH3 |
| E-567 | A-10 | Isobutyric | Isobutyric | H | | H | CH3 |
| E-568 | A-10 | Isobutyric | H | H | | H | CH3 |
| E-569 | A-10 | Valeric | Valeric | Valeric | | H | CH3 |
| E-570 | A-10 | Valeric | Valeric | H | | H | CH3 |
| E-571 | A-10 | Valeric | H | H | | H | CH3 |
| E-572 | A-10 | Isovaleric | Isovaleric | Isovaleric | | H | CH3 |
| E-573 | A-10 | Isovaleric | Isovaleric | H | | H | CH3 |
| E-574 | A-10 | Isovaleric | H | H | | H | CH3 |
| E-575 | A-11/E-16 | Butyric | H | H | H | Butyric | Butyric |
| E-576 | A-19.1 | Ac | H | | | | |
| E-577 | A-19.1 | Propionic | H | | | | |
| E-578 | A-19.1 | Butyric | H | | | | |
| E-579 | A-20.2 | Butyric | Butyric | Butyric | | | |
| E-580 | A-20.2 | Ac | Ac | Ac | | | |
| E-581 | A-20.2 | Propionic | Propionic | Propionic | | | |
| E-582 | A-12 | Valeric | H | H | H | H | |
| E-583 | A-12 | Isovaleric | H | H | H | H | |
| E-584 | A-19.3 | Valeric | H | H | | | |
| E-585 | A-19.3 | Valeric | H | Ethyl | | | |
| E-586 | A-19.3 | Valeric | Valeric | H | | | |
| E-587 | A-19.3 | Valeric | Valeric | Ethyl | | | |
| E-588 | A-19.3 | Isovaleric | H | H | | | |
| E-589 | A-19.3 | Isovaleric | H | Ethyl | | | |
| E-590 | A-19.3 | Isovaleric | Isovaleric | H | | | |
| E-591 | A-19.3 | Isovaleric | Isovaleric | Ethyl | | | |
| E-592 | A-19.3 | Butyric | H | H | | | |
| E-593 | A-19.3 | Butyric | H | Ethyl | | | |
| E-594 | A-19.3 | Butyric | Butyric | H | | | |
| E-595 | A-19.3 | Butyric | Butyric | Ethyl | | | |
| E-596 | A-17 | NO₂ | H | H | H | H | |
| E-597 | A-17 | NO₂ | NO₂ | H | H | H | |
| E-598 | A-18 | H | H | NO₂ | H | H | |
| E-599 | A-18 | H | NO₂ | NO₂ | H | H | |
| E-600 | A-11/E-16 | H | H | no cladinose | H | H | Mannose |
| | | | | ring | | | |
| E-601 | A-21 | NO₂ | H | H | H | | |
| E-602 | A-21 | NO₂ | NO₂ | H | H | | |
| E-603 | A-21 | NO₂ | NO₂ | H | NO₂ | | |

### Procedures

### Example 1

### Synthesis of E-1: Typical Nitration Procedure

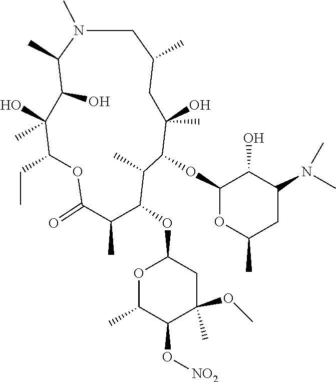

Method 1. Acetic Acid (40 mL) and Azithromycin (5 g, 6.73 mmol) were Charged in a round bottom flask. Initially, the reaction solidified which eventually during stirring, produced a homogenous solution. The resulting solution was cooled in an ice-bath. Acetic anhydride (19.8 mL, 209.5 mmol) was taken up in another reaction flask and cooled in an ice bath. To this was added dropwise, nitric acid (2.2 mL, 46.43 mmol). After complete addition, the mixture was transferred to a dropping funnel and attached to the first reaction vessel containing the macrolide. The HNO₃—Ac₂O mixture was slowly added to the reaction (ca. 1 drop per second). After complete addition, the reaction was allowed to warm to room temperature where it was stirred until reaction completion (3 h). The reaction was poured onto a stirred 200 mL ice-water. Stirring was continued until the ice is completely melted. The resulting aqueous solution was neutralized at first with a saturated solution of NaHCO₃, followed by pure solid NaHCO₃ to pH 8 to 9. The aqueous solution was extracted with DCM (5×). The DCM extracts were dried (Na₂SO₄), evaporated in vacuo. The crude product was purified by column chromatography (3:1 cyclohexane, ethyl acetate, 1% triethylamine) to get compound E-1 as a white foam (30% yield).
¹³C-NMR [ppm]: 178.5, 102.4, 95.0, 87.5, 83.2, 78.6, 74.8, 74.4, 73.6, 73.3, 71.0, 71.0, 70.1, 68.2, 65.6, 62.4, 62.0, 49.5, 44.9, 42.3, 41.2, 40.3, 36.6, 35.6, 28.6, 27.3, 26.9, 26.7, 22.0, 21.6, 21.1, 17.8, 16.2, 15.2, 11.2, 9.2, 7.7

TABLE 3
Nitration Examples

| Compound | Synthesis | | Degree of | | |
|---|---|---|---|---|---|
| Entry | Method | ALC | Substitution | Yield | MS |
| E-596 | 2 | A-17 | Mono nitro | n.d.* | 822, M + H⁺ |
| E-597 | 2 | A-17 | Di nitro | n.d.* | 867, M + H⁺ |
| | 2 | A-18 | Oxidation | n.d.* | 954, M + Na⁺ |
| E-598 | 2 | A-18 | Mono nitro | n.d.* | 999, M + Na⁺ |
| E-599 | 2 | A-18 | Di nitro | n.d.* | 1045, M + Na⁺ |
| E-601 | 2 | A-21 | Mono nitro | n.d.* | 793, M + H⁺ |
| E-602 | 2 | A-21 | Di nitro | n.d.* | 838, M + H⁺ |
| E-603 | 2 | A-21 | Tri nitro | n.d.* | 883, M + H⁺ |

n.d.* not determined

Method 1 (see Example 1) can be applied to other ALCs, and in cases where there is more than one reactive hydroxy species, selective protection is necessary.

Method 2. Hydroxyalkyl species (1 mmol) was suspended in acetonitrile in a round bottom flask while stirring (magnetic stir bar, 300 rpm). Silver nitrate (2 eq. per hydroxyl group) was added and the mixture was cooled in an ice bath. Phosgene (solution in toluene, 1 eq. per hydroxyl group) was carefully added dropwise. Immediate precipitation of silver chloride and formation of carbon dioxide indicated formation of nitro donor (caution: too quick CO₂ formation may result in strong foaming. Do not seal the flask!). After a couple of minutes, a yellow color was obtained and stirring was continued for 15 minutes. When ESI-MS indicated satisfying turn-over rate of starting materials the reaction was quenched by addition of methanol, converting excess nitro donor to volatile methyl nitrate. The system was diluted by addition of DCM and was subject to extraction with saturated sodium bicarbonate solution (3×). Separation of organic phase, drying over sodium sulfate and evaporation of any volatiles in vacuo yielded the product as colorless oil or beige to off-white foam.

### Example 2

### Synthesis of E-2

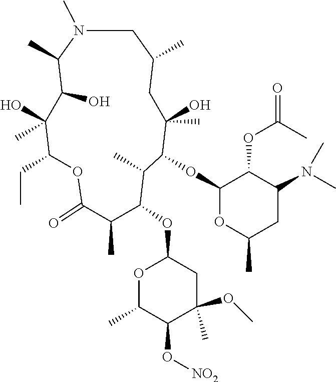

Compound E-1 (791 mg, 1.0 mmol) was taken up in 15 mL dichloromethane. Pyridine (89 mL, 1.1 mmol) was added and the resulting solution was cooled in an ice bath for approximately 10 minutes. At this point, a solution of acetic anhydride (113 ml, 1.2 mmol) in dichloromethane (15 mL) was added dropwise. The reaction was stirred continually at this temperature and then progressively warmed to room temperature where it was stirred overnight. The reaction was washed with a saturated solution of ammonium chloride (3×), water (3×) and dried over anhydrous Na₂SO₄. The solvent was evaporated in vacuo. Co-evaporation with toluene is necessary to remove residual pyridine from the system. This was followed by re-dissolving the residue in DCM and solvent evaporation twice to produce a white foam, which was dried under high-vacuum to produce E-2 (631 mg, 76%).

### Example 3

### Synthesis of E-3

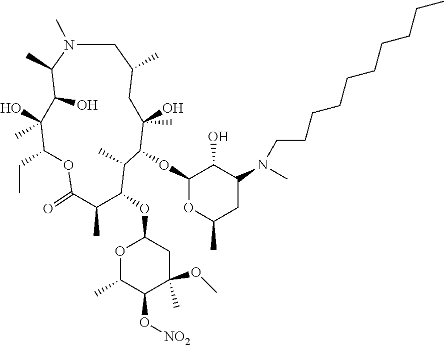

E-3 was synthesized using Method 1 starting from E-20.

### Example 4

### Synthesis of E-4

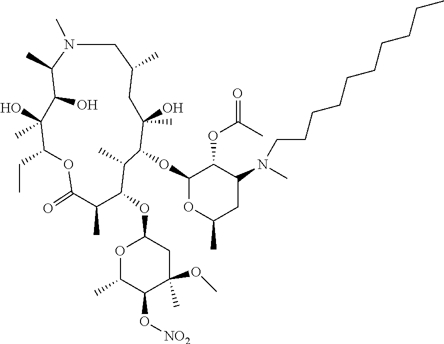

A solution of E-3 (1 mmol) and pyridine (1 mmol) in DCM (10 ml) was treated with acetic anhydride (1.5 mmol) at ambient temperature. Stirring was continued until TLC (acetone-cyclohexane 1:3) indicated complete consumption of the starting materials. The system was extracted with an aqueous solution of NH₄Cl (2×10 ml) and water (3×10 ml). After drying over Na₂SO₄ all volatile components were evaporated in vacuo, traces of pyridine species were removed by co-evaporation with toluene (2×). The product E-4 was a colorless oil (41%).

### Example 5

### Synthesis of E-5

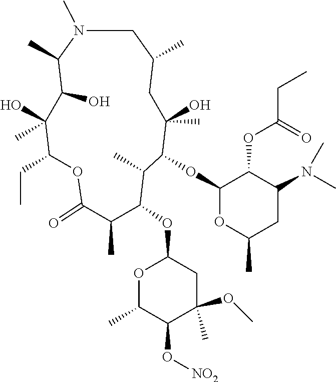

A solution of propionic acid (1.2 mmol) in 1,2-dichloroethane was treated with 1-Ethyl-3-(3-dimethyl-aminopropyl)carbodiimid (1.2 mmol) in the presence of a catalytical amount of DMAP for 30 min. at ambient temperature. Temperature was raised to 55° C., E-1 (1 mmol) was added and stirring was continued until TLC (acetone-cyclohexane 1:3) indicated complete consumption of the starting materials. The system was extracted with water (3×10 ml). After drying over Na₂SO₄ all volatile components were evaporated in vacuo, traces of pyridine species were removed by co-evaporation with toluene (2×). The crude products were purified by column chromatography (acetone-cyclohexane 1:3), yielding the product E-5 as white amorphous foam (39%).

Alternative Synthesis: Compound E-1 (310 mg, 0.39 mmol) was taken up in dichloromethane (15 mL). At which point, pyridine (32 mL, 0.39 mmol) was added. The solution was stirred for 5 minutes, at which time, propionyl anhydride (51 mL, 0.40 mmol) was added. The reaction was allowed to stir at room temperature for 72 h. An additional propionyl anhydride (0.1 eq) was added and the reaction monitored until complete disappearance of starting material was observed (MS, reaction may take up to 1 week). The reaction was washed successively with a saturated aqueous solution of NH₄Cl (3×) and H₂O (3×). The organic phase was dried over anhydrous Na₂SO₄ and evaporated in vacuo. Co-evaporation with toluene is necessary to remove residual pyridine from the system. This was followed by re-dissolving the residue in DCM and solvent evaporation twice to produce E-5 as a white foam (210 mg, 64%).

The following methodologies were used as alternative to the general method to attach propionyl groups:

Method 1: A-1 (300 mg, 0.40 mmol), was dissolved in DCM (10 mL); to this solution was added TEA (279 μL, 5 eq) and propionylchloride (175 μL, 5 eq) subsequently and the mixture was stirred overnight at room temperature. Additional TEA (112 μL, 2 eq) and propionyl chloride (70 μL, 2 eq) were added and again mixture was stirred overnight at room temperature. Once more additional TEA (112 μL, 2 eq) and propionyl chloride (70 μL, 2 eq) were added and stirring at room temperature was continued overnight. TEA (344 μL, 9 eq) and propionyl chloride (314 μL, 9 eq) were added and the mixture was stirred at room temperature for 5 days. The reaction mixture was washed with aqueous Na₂CO₃-solution (3×, 10%) and water (3×), dried, concentrated to dryness, and dried at the oil pump. ESI-MS (positive) showed tri- and tetra-propionylation.

Method 2.

Propionic acid (4 eq) was taken up in 5 mL dichloromethane (DCM). Compound A-16 (0.5 mmol) and 4-dimethylaminopyridine (DMAP) (4.4 eq) were added and the resulting solution was cooled in an ice bath for approximately 10 minutes. At this point, dicyclohexylcarbodiimide (DCC) (4.4 eq) was added slowly. The reaction was stirred continually at this temperature for 5 minutes and then progressively warmed to room temperature where it was stirred overnight. Dicyclohexylurea (DCU) that was formed during the reaction is filtered off and discarded. The filtrate was collected and then washed with a saturated solution of sodium hydrogencarbonate (3×), water (lx) and dried over anhydrous Na₂SO₄. The solvent was evaporated in vacuo. This was followed by re-dissolving the residue in a small volume of methanol. The solution was transported dropwise into ice-cold water (2× volume of methanol) and stored in the freezer overnight. The precipitated product was filtered off and dried under high-vacuum to produce a product.

TABLE 4
Propionylation Examples

| | | | | | | | MS |
|---|---|---|---|---|---|---|---|
| Compound | Synthesis | | Substituent | Reaction Condition | Degree of | | m/z |
| Entry | Method | ALC | equivalent | (i.e. Workup) | Substitution | Yield | ([M + H]⁺) |
| E-5 | 1 | A-1 | 1.1 | as described above | 1 | 64% | 850.3 |
| E-18 | See | A-1 | N/A | | 1 | 80% | 805.5 |
| | example 21 | | | | | | |
| E-419 | 4 | A-16 | 4 | | 1 | 50% (based | 791.3 |
| E-420 | | | | | 2 | on | 847.1 |
| E-421 | | | | | 3 | tri-ester | 903.0 |
| E-52 | 3 | A-1 | 18 | | 3 | Not | 917.9 |
| | | | | | | determined | |
| E-51 | | | | | 4 | | 973.8 |

### Example 6

### Synthesis of E-10

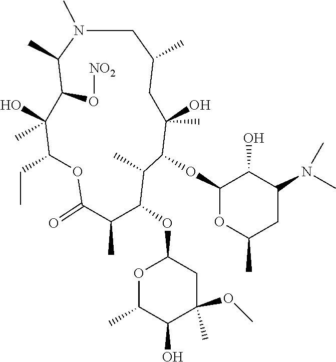

A solution of E-9 (1 mmol) in MeOH (20 ml) was vigorously stirred at 50° C. until TLC (acetone-cyclohexane 1:3) indicated complete deacetylation of the starting materials. Volatile components were removed in vacuo, the product E-10 was obtained as white amorphous foam (81%).

### Example 7

### Synthesis of E-11

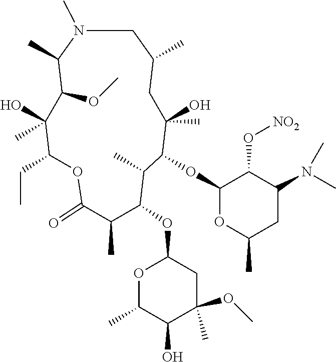

Azithromycin was nitrated as described above (Method 2). After desired mono-nitration MeOH was added to the reaction mixture and stirring was continued for one further hour. Thus in situ generated methyl nitrate acted as methylating agent transferring one methyl group to 11-O-position of the macrolide at ambient temperature. Standard aqueous workup with subsequent purification by column chromatography (acetone-cyclohexane 1:3) delivered methylated macrolide nitrate E-11 as white amorphous solid (63%, two steps).

### Example 8

### Synthesis of E-16

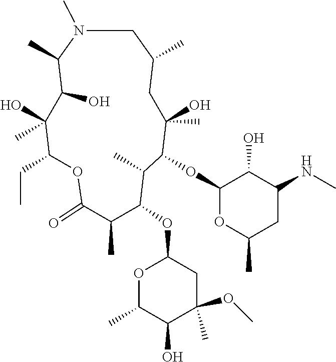

Azithromycin (20.0 g; 26.7 mmol) was dissolved in 120 ml of MeOH. NaHCO₃ (6.0 g; 71.5 mmol) was added, followed by a solution of K₂CO₃ (12.0 g; in water (80 ml; cooled down to RT), and finally iodine (6.3 g; 24.8 mmol). The mixture was stirred vigorously at ambient temperature until the dark color had disappeared. A second batch of iodine (6.3 g; 24.8 mmol) and K2CO3carbonate (4.2 g; 30 mmol) were added. The procedure [addition of iodine 6.3 g and K₂CO₃ (4.2 g; 30 mmol)] was repeated until MS showed (almost) full conversion. Sodium bisulfite was added to remove excess oxidants, and all volatiles were evaporated. The solid residue was finely ground and extensively extracted via Soxhlet extraction with acetonitrile. The extract was concentrated to ca. 75 ml and left standing at ambient temperature at least for 1 day and subsequently for another day in the fridge. All solids were collected and recrystallized from MeOH spiked with ca. 1 to 2 ml of water. Crystallization proceeded for about 3 days in an open vessel to yield 10 g (51%) of 3′-N-demethyl-azithromycin (E-16). A second crop can be obtained from the mother liquors.

### Example 9

### Synthesis of E-20

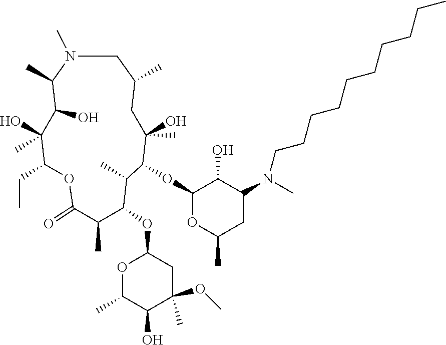

A solution of E-16 (5 mmol) in DMSO (20 ml) was treated with decyl bromide (6 mmol) at ambient temperature. Stirring continued for 12 h until TLC (acetone-cyclohexane 1:3, 1% Et₃N) indicated consumption of starting materials. The system was diluted with EtOAc (50 ml) and extracted with water (3×30 ml). The organic phase was dried over sodium sulfate. Evaporation of the solvent and drying at vacuum yielded E-20 as white amorphous foam (61%).

### Example 10

### Synthesis of E-25

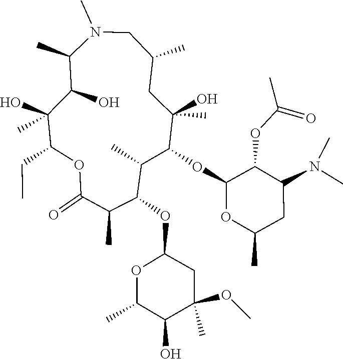

A solution of azithromycin (13 mmol) and pyridine (13 mmol) in DCM (80 ml) was cooled to 0° C. in an ice bath. A solution of acetic anhydride (14 mmol) in DCM (20 ml) was slowly added to the system. Afterwards the reaction mixture was allowed to warm up to ambient temperature and stirring was continued until TLC (acetone-cyclohexane 1:3, 1% Et₃N) indicated complete consumption of the starting materials. The system was extracted with an aqueous solution of NH₄Cl (2×50 ml) and water (3×50 ml). After drying over Na₂SO₄ all volatile components were evaporated in vacuo, traces of pyridine were removed by coevaporation with toluene (2×). The product E-25 was a white amorphous solid (67%).

### Example 11

### Synthesis of E-22

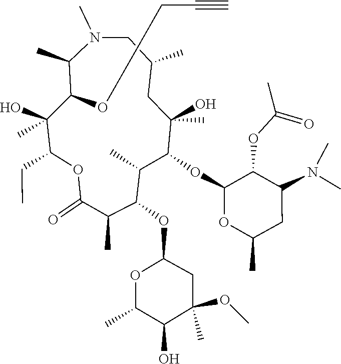

A solution of E-25 (4 mmol) in dry THF (30 ml) was cooled to 0° C. in an ice bath. A solution of propargyl bromide (4.4 mmol, 80% in toluene) was added slowly to the system. Afterwards the reaction mixture was allowed to warm up to ambient temperature and stirring was continued until TLC (acetone-cyclohexane 1:3, 1% Et₃N) indicated complete consumption of the starting materials. The system was diluted with EtOAc (50 ml) and extracted with water (3×50 ml). After drying over Na₂SO₄ volatile components were evaporated in vacuo. Purification by column chromatography (acetone-cyclohexane 1:3, 1% Et₃N) furnished E-22 as white amorphous powder (57%).

### Example 12

### Synthesis of E-12

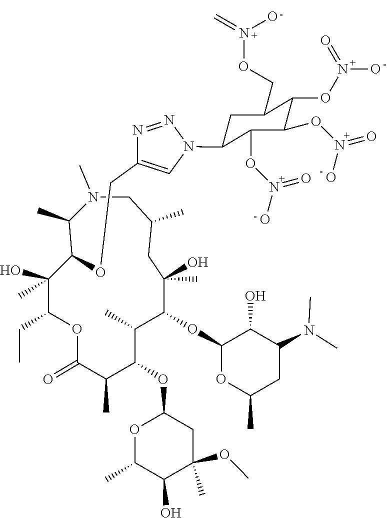

A solution of E-22 (0.5 mmol), I-5 (0.5 mmol) and DIPEA (1 mmol) in toluene (5 ml) was treated with triethylphosphito copper(I) iodide complex (0.05 mmol) at ambient temperature. Stirring was continued until TLC (ethyl acetate-cyclohexane 1:1) indicated complete consumption of the starting materials. The mixture was concentrated in vacuo and purified by column chromatography (acetone-cyclohexane 1:1→acetone). The product E-12 was obtained as colorless foam (32%).

### Example 13

### Synthesis of E-23

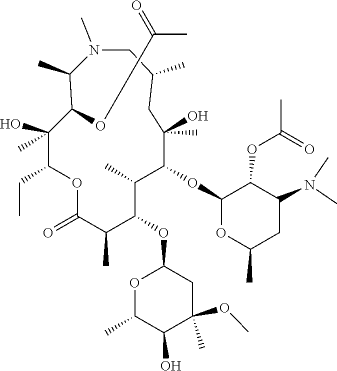

A solution of E-25 (5 mmol) and pyridine (5 mmol) in DCM (40 ml) was treated with acetic anhydride (7 mmol) at ambient temperature. Stirring was continued until TLC (acetone-cyclohexane 1:3, 1% Et₃N) indicated complete consumption of the starting materials. The system was extracted with an aqueous solution of NH₄Cl (2×20 ml) and water (3×30 ml). After drying over Na₂SO₄ all volatile components were evaporated in vacuo, traces of pyridine species were removed by co-evaporation with toluene (2×). The product E-23 was a white amorphous powder (54%).

### Example 14

### Synthesis of E-17

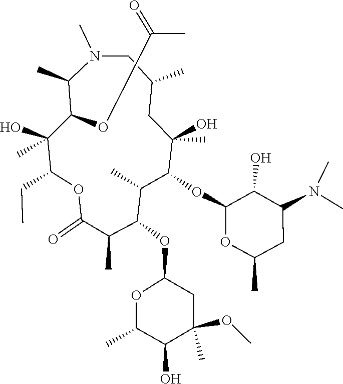

A solution of E-23 (2 mmol) in MeOH (30 ml) was vigorously stirred at 50° C. until TLC (acetone-cyclohexane 1:3, 1% Et₃N) indicated complete deacetylation of the starting materials. Volatile components were removed in vacuo, the product E-17 was obtained as white amorphous foam (73%).

Alternative Synthesis: Compound E-2 (619 mg, 0.74 mmol) was charged in a round bottom flask. To this was added a solution of acetic acid/methanol (2:1, 15 mL). To the stirred solution, was added Zn powder (368 mg, 5.63 mmol, 7.6 eq). The resulting suspension was stirred at room temperature and progressively monitoring the disappearance of the starting material (approx. 3 h). The suspension was filtered and the filtrate evaporated in vacuo. The residue was taken up in dichloromethane (15 mL) producing some white precipitate. The precipitate was filtered off. The dichloromethane filtrate was washed with 10% aqueous Na₂CO₃ solution (2×), water (lx) and dried over anhydrous Na₂SO₄. The solvent was evaporated in vacuo producing E-17 as a white solid (475 mg, 81% yield).

### Example 15

### Synthesis of E-26

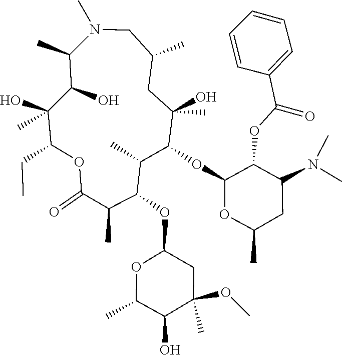

A solution of azithromycin (13 mmol) and pyridine (13 mmol) in DCM (80 ml) was cooled to 0° C. in an ice bath. A solution of benzoyl chloride (14 mmol) in DCM (20 ml) was slowly added to the system. Afterwards the reaction mixture was allowed to warm up to ambient temperature and stirring was continued until TLC (acetone-cyclohexane 1:3, 1% Et₃N) indicated complete consumption of the starting materials. The system was extracted with an aqueous solution of NH₄Cl (2×50 ml) and water (3×50 ml). After drying over Na₂SO₄ all volatile components were evaporated in vacuo, traces of pyridine species were removed by co-evaporation with toluene (2×). Column chromatography (acetone-cyclohexane 1:3, 1% Et₃N) yielded the product E-26 as a white amorphous foam (44%).

### Example 16

### Synthesis of E-13

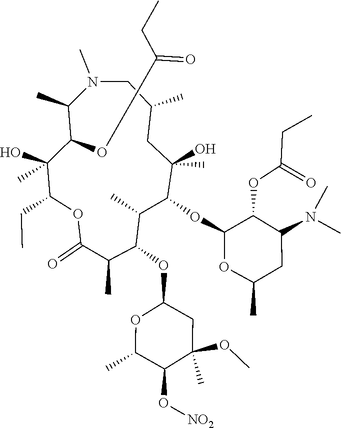

A solution of E-5 (1 mmol) and propionic acid (1.2 mmol) in 1,2-dichloroethane (3 ml) was treated portion wise with EDCI (3×0.8 mmol) and DMAP (1 mmol). The mixture was heated to 55° C. and stirred for 6 days. After TLC (acetone-cyclohexane 1:3) indicated complete consumption of the starting materials the mixture was diluted with EtOAc (30 ml) and extracted with water (3×20 ml). After drying over Na₂SO₄ the organic phase was evaporated and the remains were purified by column chromatography (acetone-cyclohexane 1:3). The product E-13 was obtained as colorless amorphous foam (42%).

### Example 17

### Synthesis of E-14

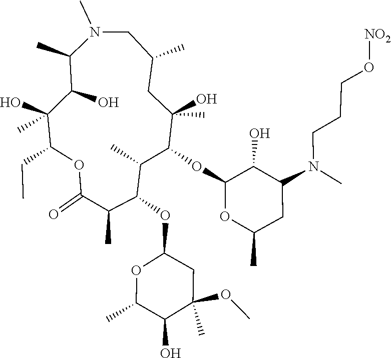

3′-N-desmethyl-azithromycin (E-16/I-1) (300 mg; 0.41 mmol) and 1-bromo-3-nitrooxy-propane^{[17]} (90 mg, 0.49 mmol) were dissolved in dry DMSO (1.2 ml). The mixture was shaken at 23° C. for 3 hours. Afterwards additional 1-bromo-3-nitrooxy-propane (90 mg, 0.49 mmol) was added and shaking was continued for another hour. Once again, 1-bromo-3-nitrooxy-propane (90 mg, 0.49 mmol) was added, the reaction mixture was shaken for additional 90 min., and then kept in the freezer (−16° C.) over night. The next morning additional 1-bromo-3-nitrooxy-propane (90 mg, 0.49 mmol) was added and the mixture was shaken for one hour. Water and DCM were added; after extraction, the organic phase was dried (Na₂SO₄) and concentrated to dryness (without heating). The crude product was purified by column chromatography (eluent:CHCl₃:Isopropanol:NH₃ (7 M in MeOH 30:1:1).

### Example 18

### Synthesis of (I-5)

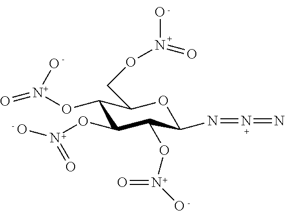

Azido-β-D-Glucopyranoside is synthesized from corresponding sugar acetate as is known to literature^{[16]}. After removal of any protective groups sugar azide is nitrated following above procedure. Due to its high explosive risk the substance is always kept as DCM solution and is stashed in the refrigerator.

### Example 19

### Synthesis of E-24

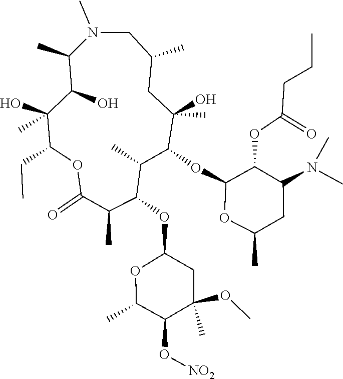

Compound E-1 (405 mg, 0.51 mmol) was taken up in dichloromethane (15 mL). The resulting solution was cooled to 0° C. After 5 minutes, butyryl chloride (60 mL, 61.8 mg, 0.58 mmol, 1.1 eq.) was added. The reaction was stirred for 10 min. at this temperature, at which point, the reaction was allowed to warm to room temperature, where it was stirred until reaction completion (2 h, or upon continuous monitoring). The reaction was washed with a 10% Na₂CO₃ aq. solution (3×) followed by H₂O (3×), dried over anhydrous Na₂SO₄ and evaporated in vacuo to give E-24 as a white foam (332 mg, 75% yield).

### Example 20

### Synthesis of E-30

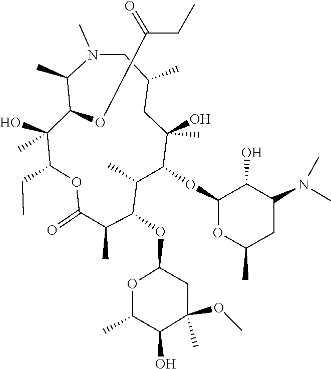

Compound E-5 (CSY 1076) (126 mg, 0.15 mmol) was charged in a round bottom flask. To this was added a solution of acetic acid/methanol (2:1, 12 mL). To the stirred solution, was added Zn powder (74 mg, 1.12 mmol, 7.6 eq). The resulting suspension was stirred at room temperature and progressively monitoring the disappearance of the starting material (approx. 3 h). The suspension was filtered and the filtrate evaporated in vacuo. The residue was taken up in dichloromethane (10 mL) producing some white precipitate. The precipitate was filtered off. The dichloromethane filtrate was washed with 10% aqueous Na₂CO₃ solution (2×), water (1×) and dried over anhydrous Na₂SO₄. The solvent was evaporated in vacuo producing E-18 as a white solid (96 mg, 80% yield).

The reduction conditions for removal of the NO₂ group was applied to the syntheses of E-19 from E-24 providing the desired product in 78% yield.

### Example 21

### Synthesis of E-35

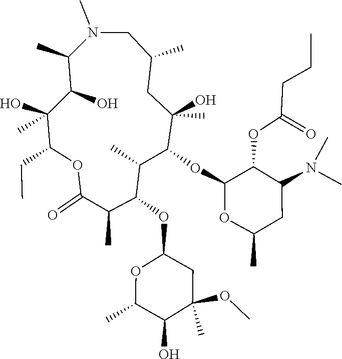

Compound E-24 CSY 4636 (100 mg, 0.12 mmol) was charged in a round bottom flask. To this was added a solution of acetic acid/methanol (2:1, 12 mL). To the stirred solution, was added Zn powder (88 mg, 1.35 mmol, 11.6 eq). The resulting suspension was stirred at room temperature and progressively monitoring the disappearance of the starting material (approx. 3 h). The suspension was filtered and the filtrate evaporated in vacuo. The residue was taken up in dichloromethane (15 mL) producing some white precipitate. The precipitate was filtered off. The dichloromethane filtrate was washed with 10% aqueous Na₂CO₃ solution (2×), water (lx) and dried over anhydrous Na₂SO₄. The solvent was evaporated in vacuo producing E-19 as transparent gel (74 mg, 78% yield).

### Example 22

### Synthesis of E-81

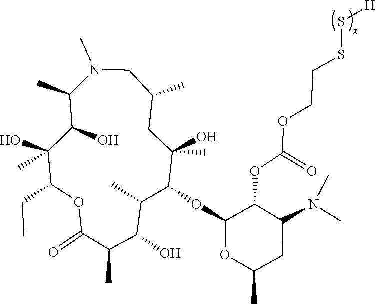

250 mg of 2′-(2-Mercaptoethoxy)carbonyl-3-decladinosylazithromycin and 250 mg of ammonium polysulfide are mixed with 10 ml of degassed and argonized glacial acetic acid and stirred with exclusion of oxygen for 12 h. All volatiles are removed in vacuo, and the residue is extracted with oxygen free saturated aqueous sodium hydrogen carbonate solution 3 times. The residue is washed with water (oxygen free), dried in vacuo and used as such.

### Example 23

### Synthesis of E-74

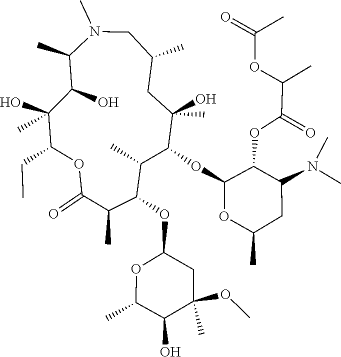

380 mg of Azithromycin are dissolved with 20 ml of DMF and cooled in an ice bath. 41 μl of pyridine and then 85 mg (1.1 eq.) of rac-2-acetoxy propionyl chloride, dissolved with 1 ml of dichloromethane, are added and the mixture is allowed to warm up to room temperature with stirring. When mass spectrometry indicates consumption of the macrolide, the mixture is diluted with 50 ml of ethyl acetate, extracted twice with water, 3 times with saturated aqueous sodium hydrogen carbonate solution, once again with water and brine, each, and dried over sodium sulfate. After evaporation and vacuum drying, the diastereomeric mixture (1:1) of target compound E-74 remains as a slightly yellowish foam.

Yield: 385 mg

MS: m/z=863.5 ([M+H]⁺)

The same procedure can be applied in preparing E-77, E-83, E-84, E-85 and E-86.

TABLE 5
Typical Products from the Acylation Procedures

| Entry | Acylating agent | Product structure | Yield [%] | ([M + H]⁺ |
|---|---|---|---|---|
| E-83 | O-Phenyl chlorothionoformate | 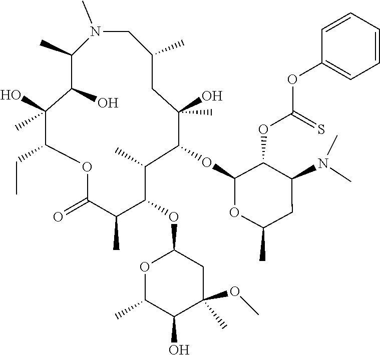 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2OC(=S)Oc2ccccc2)[C@](C)(O)C[C@@H](C)CN(C)[C@H](C)[C@@H](O)[C@]1(C)O] | 66 | 885.7 |
| E-85 | 2-Bromoethylchloroformate | 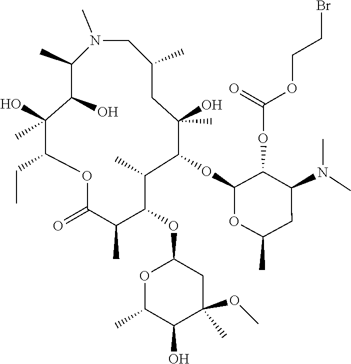 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2OC(=O)OCCBr)[C@](C)(O)C[C@@H](C)CN(C)[C@H](C)[C@@H](O)[C@]1(C)O] | 82 | 899.6 |
| E-84 | Hexanoylchloride | 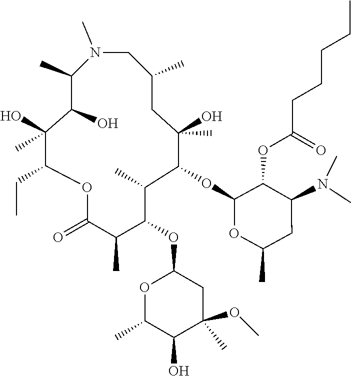 [SMILES: CCCCCC(=O)O[C@H]1[C@H](O[C@@H]2[C@@H](C)[C@H](O[C@H]3C[C@@](C)(OC)[C@@H](O)[C@H](C)O3)[C@@H](C)C(=O)O[C@H](CC)[C@@](C)(O)[C@H](O)[C@@H](C)N(C)C[C@H](C)C[C@@]2(C)O)O[C@H](C)C[C@@H]1N(C)C] | 93 | 847.6 |
| E-77 | Lipoyl chloride | 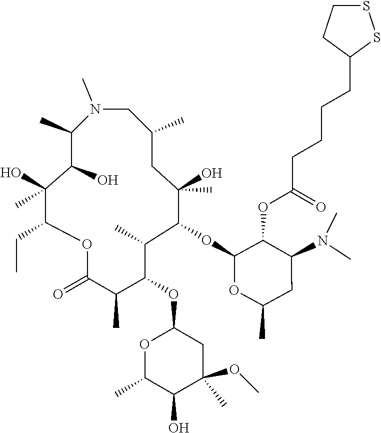 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2OC(=O)CCCCC2CCSS2)[C@](C)(O)C[C@@H](C)CN(C)[C@H](C)[C@@H](O)[C@]1(C)O] | 72 | 936.8 |
| E-86 | Allylchloroformate | 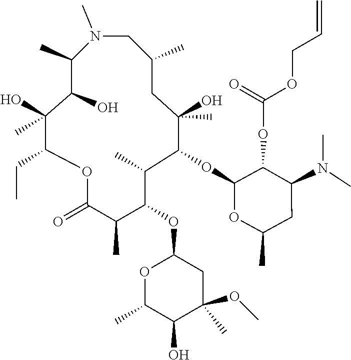 [SMILES: C=CCOC(=O)O[C@H]1[C@H](O[C@@H]2[C@@H](C)[C@H](O[C@H]3C[C@@](C)(OC)[C@@H](O)[C@H](C)O3)[C@@H](C)C(=O)O[C@H](CC)[C@@](C)(O)[C@H](O)[C@@H](C)N(C)C[C@H](C)C[C@@]2(C)O)O[C@H](C)C[C@@H]1N(C)C] | 82 | 833.5 |
| E-540 | O-Phenyl chlorothionoformate | 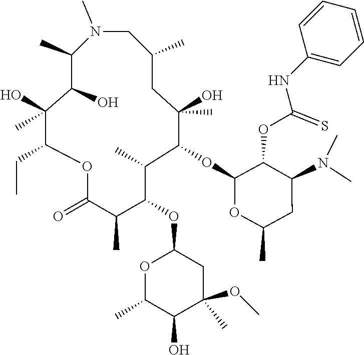 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2OC(=S)Nc2ccccc2)[C@](C)(O)C[C@@H](C)CN(C)[C@H](C)[C@@H](O)[C@]1(C)O] | 23 | 884 |
| E-26 | Benzoyl | 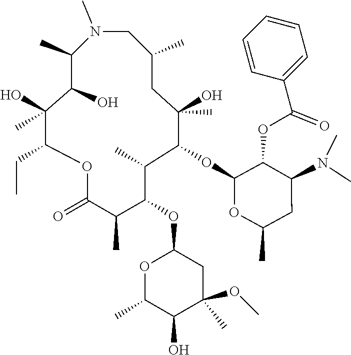 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2OC(=O)c2ccccc2)[C@](C)(O)C[C@@H](C)CN(C)[C@H](C)[C@@H](O)[C@]1(C)O] | 44 | 853 |

### Example 24

General Procedure: Synthesis of n-Butyryl-Propanolol, Compound E-133

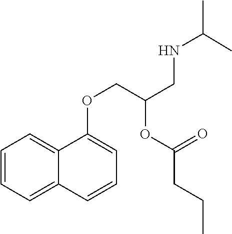 [SMILES: CCCC(=O)OC(CNC(C)C)COc1cccc2ccccc12]

Propranolol.HCl (200 mg, 0.68 mmol) was taken up in dichloromethane (4 mL). To this was added dropwise butyryl chloride (69 μL, 0.71 mmol) and the reaction was stirred at room temperature for 1 hour. To the reaction was added triethylamine (194 μL, 1.4 mmol). After 30 min, additional butyryl chloride (30 μL, 0.3 mmol) was added. Reaction was monitored by the disappearance of starting material. The reaction was stopped after 20 min by the addition of 10 mL 10% aqueous Na₂CO₃ solution. The two phases were stirred for 5 min separated. The organic layer was washed successively with 10% aqueous Na₂CO₃ (1×), H₂O (1×) and saturated aq. NaCl (1×), dried with Na₂SO₄ and evaporated in vacuo to get an oil film. 5 mL HCl in Et₂O (2M) and 1 mL MeOH was added and stirred for 5 min, then evaporated in vacuo and dried with the vacuum pump under nitrogen for 1 hour to get a brown oil (yield 91%).

### Example 25

General Procedure: Synthesis of n-Butyryl-Hydroxychloroquine, Compound E-89

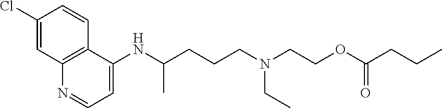 [SMILES: CCCC(=O)OCCN(CC)CCCC(C)Nc1ccnc2cc(Cl)ccc12]

Hydroxychloroquine sulfate (1099 mg, 2.53 mmol) was charged into a round bottom flask. H₂O (10 mL) and dichloromethane (10 mL) were added. Pyridine (412 μL, 5.1 mmol) was added and the reaction stirred vigorously for 5 min. Butyric anhydride (420 μL, 2.65 mmol) was added and the reaction stirred at room temperature for 3 h. The phases were separated and the dichloromethane layer was washed successively with a saturated aqueous NH₄Cl solution (2×15 mL), H₂O (2×10 mL), dried over Na₂SO₄ and evaporated in vacuo. Co-evaporation with toluene is necessary to remove residual pyridine from the system. This was followed by re-dissolving the residue in DCM and solvent evaporation twice to produce a yellow oil (93 mg, 9% yield).

This procedure can be applied for the synthesis of the following compounds E-87 to E-88, E-90 to E-97.

### Example 26

Typical methylation reaction of Hydroxychloroquine or Propranolol was achieved using Eschweiler-Clarke-methylation reaction. Acylation reactions were carried out using typical procedures described above.

### Example 27

Compounds, prepared by reacting a carrier molecule with acylating agents.

These carrier molecules are prepared by reacting symmetric or unsymmetric di- or poly-epoxides with secondary amines, thus containing the common structural element of 2 or more alcohols, vicinally neighbored by a tertiary amine.

Alternatively, carrier molecules can be prepared by reacting epoxides with diethanolamine. The reaction products are containing 2-hydroxy tertiary amines.

TABLE 6
Examples for di- or polyepoxides

| | | | No. of |
|---|---|---|---|
| | | | epoxide |
| Entry | Name | CAS-No. | functions |
| A | 1,3-Butadiene diepoxide | 1464-53-5 | 2 |
| B | 1,4-Butandiole diglycidylether | 2425-79-8 | 2 |
| C | N,N-Diglycidylaniline | 2095-06-9 | 2 |
| D | Resorcinol diglycidylether | 101-90-6 | 2 |
| E | Ethylen glycol diglycidylether | 2224-15-9 | 2 |
| F | 1,7-octadienediepoxide | 2426-07-5 | 2 |
| G | Diglycidyl ether | 2238-07-5 | 2 |
| H | 1,2,4,5,9,10-Triepoxydecane | 52338-90-6 | 3 |
| I | N,N-Diglycidyl-4-glycidyloxyaniline | 5026-74-4 | 3 |
| J | Poly(ethylene glycol) diglycidyl ether | 72207-80-8 | 2 |
| | (average Mₙ 500) | | |
| K | Glycerol diglycidylether | 72207-80-8 | 2 |
| L | 4,4′-Methylenebis(N,N- | 28768-32-3 | 4 |
| | diglycidylaniline) | | |
| M | Bis[4-(glycidyloxy)phenyl]methane | 2095-03-6 | 2 |

TABLE 7
Examples for secondary amines

| Entry | Name | CAS-No. |
|---|---|---|
| 1 | Dimethylamine | |
| 2 | Morpholine | |
| 3 | 4-Methylpiperazine | |
| 4 | Piperidine | |
| 5 | 1,2,3,4-Tetrahydroisoquinoline | 91-21-4 |
| 6 | Diethylamine | |
| 7 | Dioctylamine | |
| 8 | Diethanolamine | |
| 9 | Sarcosine methyl ester hydrochloride | 13515-93-0 |
| 10 | (R)-pyrrolidine-2-carboxylic acid methyl ester | |
| | (Prolin methylester) | |
| 11 | Ethyl 1,4-diazepan-1-ylacetate dihydrochloride | |

TABLE 8
Structural examples for carrier molecules

| Combination of | |
|---|---|
| poly-epoxide | |
| and amine | Structural Example |
| H1 | 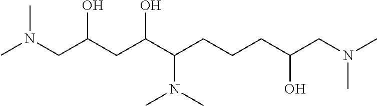 |
| G5 | 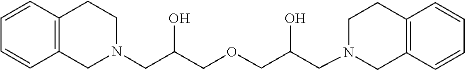 [SMILES: OC(COCC(O)CN1CCc2ccccc2C1)CN1CCc2ccccc2C1] |
| C8 | 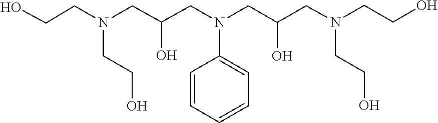 [SMILES: OCCN(CCO)CC(O)CN(CC(O)CN(CCO)CCO)c1ccccc1] |
| A-7-C | 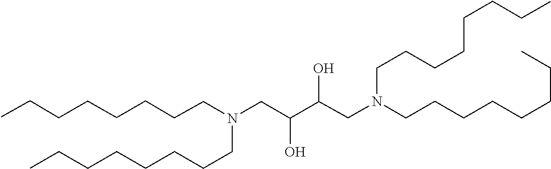 [SMILES: CCCCCCCCN(CCCCCCCC)CC(O)C(O)CN(CCCCCCCC)CCCCCCCC] |
| B2 | 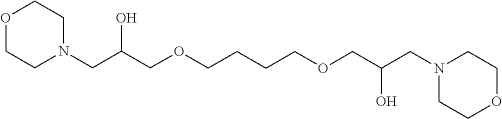 [SMILES: OC(COCCCCOCC(O)CN1CCOCC1)CN1CCOCC1] |

The 2-aminoalcohols of these carriers can be esterified to short chain carboxylic acids or nitric acid. One molecule can contain esters of different of these acids. Examples for short chain carboxylic acids are:

Acetic acid, propionic acid, butyric acid, isobutyric acid, valeric acid, 2-methylbutyric acid, 3-methylbutryric acid, lactic acid, pyruvic acid, 3-phenylpropionic acid, succinic acid, maleic acid, fumaric acid, malic acid, lactic acid butyrate (lactic acid butanoate), 2-acetoxy propionic acid, mandelic acid, benzoic acid.

Structural Examples of Such Esters are:

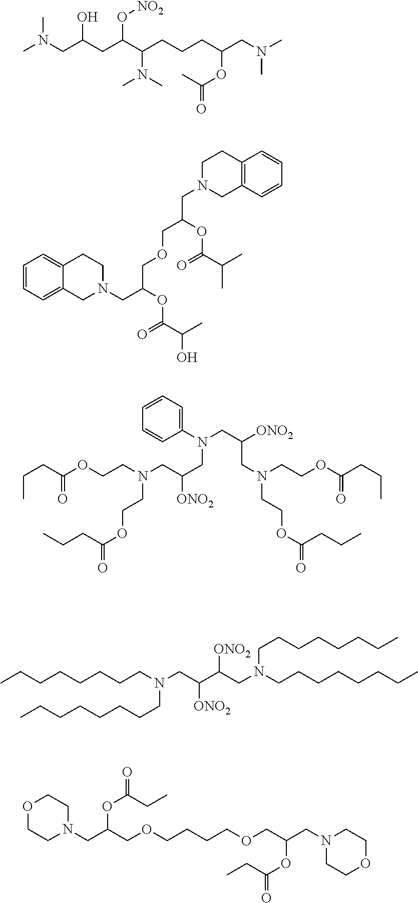

Alternatively these carrier molecules are prepared by reacting symmetric or unsymmetric di- or polyamines with epoxides, thus containing the common structural element of 2 or more alcohols, vicinally neighboured by a tertiary amine.

TABLE 9
Examples for di- or polyamines

| | | | No. of |
|---|---|---|---|
| | | | reactive |
| Entry | Name | CAS. No. | amines |
| N | Spermine | 71-44-3 | 4 |
| O | Spermidine | 124-20-9 | 3 |
| P | Piperazine | 110-85-0 | 2 |
| Q | 1-(2-Aminoethyl)piperazine | 140-31-8 | 2 |
| R | L-Lysine | 56-87-1 | 2 |
| S | Homopiperazine | 505-66-8 | 2 |
| T | 1,3-Diamino-2-propanol | 616-29-5 | 2 |
| U | 1,3,5-Triamino-1,3,5-trideoxy-cis-inositol | 6988-69-8 | 3 |
| | trihydrochloride | | |
| V | 1,2,3,4-Tetrahydroquinoxaline | 3476-89-9 | 2 |
| W | Tetraethylenepentamine | 112-57-2 | 5 |

TABLE 10
Examples for epoxides

| Entry | Name | CAS No. |
|---|---|---|
| 12 | Propylene oxide | 75-56-9 |
| 13 | Cyclohexen oxide | 286-20-4 |
| 14 | Ethyl 2,3-epoxypropionate | 4660-80-4 |
| 15 | Styrene oxide | 96-09-3 |
| 16 | Glycidol | 556-52-5 |

TABLE 11
Structural examples for carrier molecules

| Combination of | |
|---|---|
| poly-epoxide | |
| and amine | Structural Example |
| Q12 | 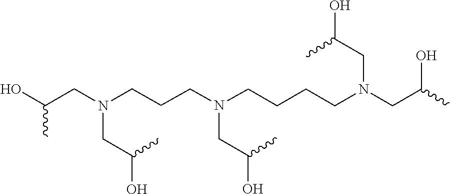 |
| Q13 | 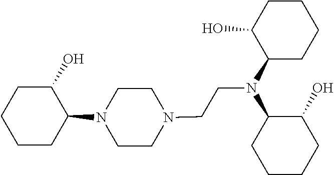 [SMILES: O[C@@H]1CCCC[C@H]1N(CCN1CCN([C@H]2CCCC[C@@H]2O)CC1)[C@@H]1CCCC[C@H]1O] |
| R16 | 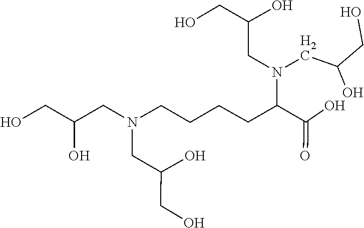 |
| S14 | 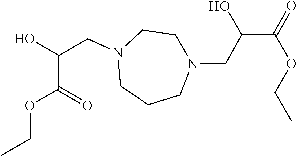 [SMILES: CCOC(=O)C(O)CN1CCCN(CC(O)C(=O)OCC)CC1] |
| T15 | 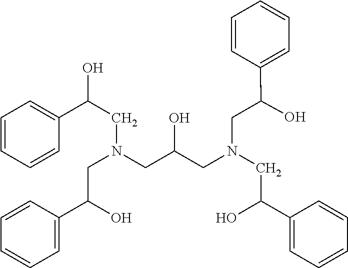 [SMILES: OC(CN(CC(O)c1ccccc1)CC(O)c1ccccc1)CN(CC(O)c1ccccc1)CC(O)c1ccccc1] |

The 2-aminoalcohols of these carriers can be esterified to short chain carboxylic acids or nitric acid. One molecule can contain esters of different of these acids. Examples for short chain carboxylic acids are:

Acetic acid, propionic acid, butyric acid, isobutyric acid, valeric acid, 2-methylbutyric acid, 3-methylbutryric acid, lactic acid, pyruvic acid, 3-phenylpropionic acid, succinic acid, maleic acid, fumaric acid, malic acid, lactic acid butyrate (lactic acid butanoate), 2-acetoxy propionic acid, mandelic acid, benzoic acid.

Structural examples of such esters are:

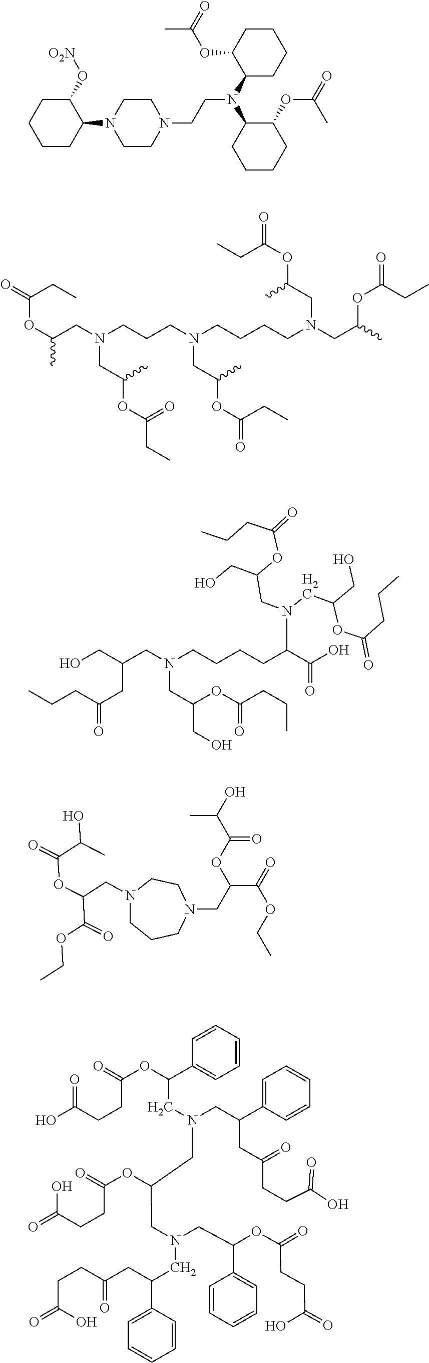

### Example 28

### Synthesis of rac. 2′-Deoxy-2′-S-thioacetyl Propranolol^{[18]}

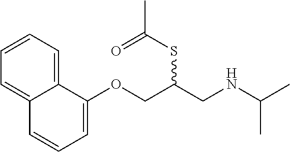 [SMILES: CC(=O)SC(CNC(C)C)COc1cccc2ccccc12]

Diethyl azodicarboxylate (DEAD, 10 mmol) is added to a stirred (magnetic stirrer, 300 rpm) solution of triphenylphosphin (10 mmol) in dry THF (25 mL) at 0° C. and treatment is continued for 30 min. Propranolol (5 mmol) and thioacetic acid (10 mmol) both dissolved in THF (10 mL) are added dropwise and stirring is continued for 1 h at 0° C. and further 2 h at ambient temperature. Any precipitates are filtered off, the remaining solution is concentrated in vacuo and desired product is isolated by column chromatography (silica gel, cyclohexane—ethyl acetate).

### Synthesis of rac. 2′-Deoxy-2′-S-thio Propranolol Sodium Salt

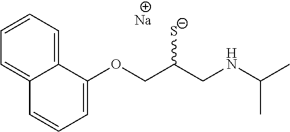 [SMILES: CC(C)NCC([S-])COc1cccc2ccccc12 ; [Na+]]

rac. 2′-Deoxy-2′-S-thioacetyl propranolol (10 mmol) is dissolved in methanol (20 mL) while stirring (magnetic stirrer, 300 rpm) and sodium methoxide (10 mmol) is added at 0° C. The system is allowed to warm up to ambient temperature and treatment is continued until TLC (cyclohexane—ethyl acetate) indicates full conversion of starting materials. Afterwards the reaction mixture is rinsed into ice cold diethyl ether (100 mL). Any precipitates are filtered off and are dried in vacuo to yield a white to slightly yellow product.

### Example 29

### Synthesis of rac. 2-O-Nitrolactic Acid

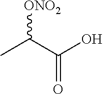

Lactic acid (10 mmol) is suspended in acetonitrile (20 mL) in a 3-necked round bottom flask and is cooled to 0° C. in an ice bath while stirring (300 rpm). Diphosgene is added (5 mmol) followed by careful dropwise addition of silver nitrate solution (20 mmol, dissolved in acetonitrile). The mixture is stirred for 30 min at 0° C., subsequently is allowed to warm up to ambient temperature and stirring is continued for further 30 min. Afterwards any precipitates are filtered off and the mixture is carefully concentrated in vacuo. As crude products are likely to be explosive compounds the system was not fully dried but taken up in THF (10 mL) to be immediately used in the following step.

### Synthesis of rac. 2′-O-(2-O-Nitrolactyl) propranolol—E-142

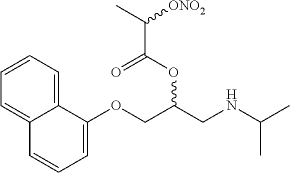

Diethyl azodicarboxylat (DEAD, 10 mmol) is added to a stirred (magnetic stirrer, 300 rpm) solution of triphenylphosphin (10 mmol) in dry THF (25 mL) at 0° C. and treatment is continued for 30 min. Propranolol (5 mmol) and rac. 2-O-nitrolactic acid (10 mmol) both dissolved in THF (10 mL) are added dropwise and stirring is continued for 1 h at 0° C. and further 2 hours at ambient temperature. Any precipitates are filtered off, the remaining solution is concentrated in vacuo and desired product is isolated by column chromatography (silica gel, cyclohexane—ethyl acetate).

### Example 30

### rac. Bis-(2′-Deoxy-2′-S—S-disulfido propranolol) (putative)^{[19]}

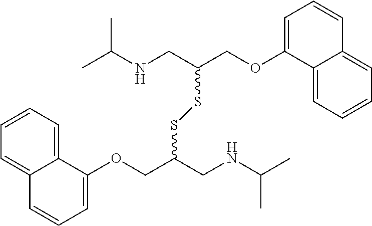 [SMILES: CC(C)NCC(COc1cccc2ccccc12)SSC(CNC(C)C)COc1cccc2ccccc12]

A round bottom flask is charged with ethyl acetate (10 mL), graphite (3 g), iodine (0.5 mmol) and cerium(III) chloride heptahydrate (1 mmol). The mixture is stirred for 10 min at ambient temperature, followed by addition of rac. 2′-deoxy-2′-S-thio propranolol sodium salt (10 mmol). Treatment is continued until TLC (cyclohexane—ethyl acetate) indicates full conversion of starting materials. After completion the system is further diluted with additional ethyl acetate (250 mL) and is washed with a saturated solution of aqueous sodium thiosulfate, water, and is dried over sodium sulfate. Upon filtration any volatiles are removed in vacuo and the residue is subject to column chromatography (silica gel, cyclohexane—ethyl acetate).

Mixed disulfides may also be accessible in this way but require slight alterations.

### Example 31—Typical Example of Synthesizing ALC Cores A-8 and A-9

### Synthesis of 2-(4-pyridyl)-3-amino-4-(3-methoxyphenyl)carbonyl pyrazole

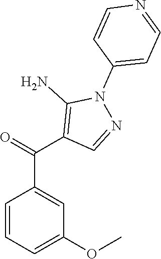 [SMILES: COc1cccc(C(=O)c2cnn(-c3ccncc3)c2N)c1]

10.96 g of 4-(N-phenyl)amino-3-(3-methoxyphenyl)carbonylacrylonitrile and 6.27 g of 4-pyridylhydrazine hydrochloride are combined with 6.2 ml of triethylamine in 140 ml of ethanol, flushed with argon and heated to reflux for 5 h. The mixture is concentrated to 50 ml and diluted with 200 ml of cyclohexane. The precipitate is filtered off and washed with diethyl ether, until no further colour is extracted any more. The remaining solid is dissolved with a mixture of dichloromethane and water (300 ml each). The organic phase is dried with brine and sodium sulfate and concentrated to dryness.

Yield: 7.07 g (61%); MS: m/z=295 ([M+H]+)

### Synthesis of 2-(4-pyridyl)-3-amino-4-(3-hydroxyphenyl)carbonyl pyrazole

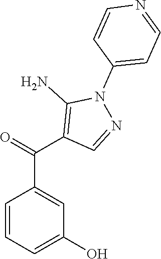 [SMILES: Nc1c(C(=O)c2cccc(O)c2)cnn1-c1ccncc1]

2.6 g of 2-(4-Pyridyl)-3-amino-4-(3-methoxyphenyl)carbonylpyrazole are suspended in 10 ml of a solution of 33% hydrobromic acid in acetic acid. The mixture is heated to 70° C. for 16 h. After cooling, the reaction mix is poured into 150 ml of water. The precipitate is filtered off, washed with saturated aqueous sodium hydrogen carbonate solution (twice) and water, and dissolved in 10 ml of a solution of ammonia in methanol (7M). After 30 min, all volatiles are removed by evaporation, and the residue is dissolved in 50 ml of boiling methanol. The product is precipitated by pouring into 250 ml of water. Filtration and drying yield 2.35 g of an off white powder.

### Synthesis of 2-(4-pyridyl)-3-amino-4-(3-[2,3-dihydroxypropyloxy]phenyl)-carbonyl pyrazole

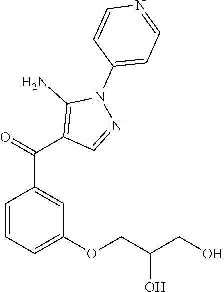 [SMILES: Nc1c(C(=O)c2cccc(OCC(O)CO)c2)cnn1-c1ccncc1]

2.09 g of 2-(4-Pyridyl)-3-amino-4-(3-hydroxyphenyl)carbonyl pyrazole are dissolved with 25 ml of dimethylformamide. 3.5 g of potassium carbonate and 580 mg of glycidol are added. The mixture is kept stirring at 60° C. for 18 h. The reaction mixture is partitioned between water and ethyl acetate. The organic phase is washed with water, dried with brine and sodium sulfate, and concentrated i.v. The residue is subjected to preparative HPLC to yield 1.4 g of the product.

### Synthesis of 2-(4-pyridyl)-3-amino-4-(3-[2,3-di{butyroyloxy}propyloxy]phenyl)carbonyl pyrazole E-199

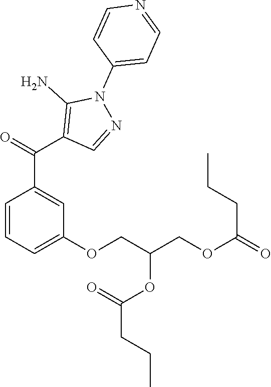 [SMILES: CCCC(=O)OCC(COc1cccc(C(=O)c2cnn(-c3ccncc3)c2N)c1)OC(=O)CCC]

500 mg of 2-(4-pyridyl)-3-amino-4-(3-[2,3-dihydroxypropyloxy]phenyl)carbonylpyrazole are dissolved with 5 ml of pyridine. 500 μl of butyric acid anhydride are added, and the mixture is stirred at 50° C. over night. 1 ml of methanol is added, and the mixture is stirred for further 30 min. The reaction is allowed to reach room temperature and partitioned between water and ethyl acetate. The organic phase is extracted with water 5 times, then with brine and dried over sodium sulfate. After evaporation of all volatiles, the product is purified by chromatography over silica gel.

Yield: 290 mg

The same conditions can be applied to synthesis of 2-(4-fluorophenyl)-3-amino-4-(3-[2,3-di{butyroyloxy}propyloxy]phenyl)carbonylpyrazole E-210

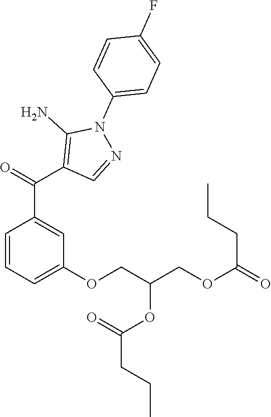 [SMILES: CCCC(=O)OCC(COc1cccc(C(=O)c2cnn(-c3ccc(F)cc3)c2N)c1)OC(=O)CCC]

### Example 32

### Synthesis of Chenodeoxycholic Acid Azithromycin-2′-ester

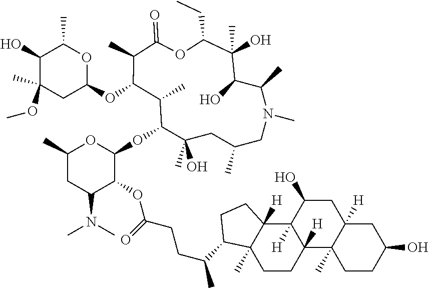

1 g of Chenodeoxy cholic acid is dissolved with 50 ml of dry dichloromethane and cooled in an ice bath. 500 mg of carbonyl diimidazole are added, and the mixture is stirred for 2 h, while reaching room temperature. 2 g of Azithromycin are added, and the mixture is stirred for 72 h. The mixture is extracted with water (3×) and then with 5% citric acid. The citric acid phase is washed with dichloromethane (2×). It is then vigorously stirred with ethyl acetate, while portions of sodium hydrogencarbonate are added, so that gas evolution is under control. When no gas is developed any more, the organic phase is isolated, washed with brine and dried over sodium sulfate. Concentration and chromatography with a gradient starting at 10% of acetone in cyclohexane (always containing 0.2% of triethylamine) yields 350 mg of the desired product.

### Example 33

### Synthesis of 2′-(succinyl-1-hydroxymethylferrocene)-11-nitro-Azithromycin

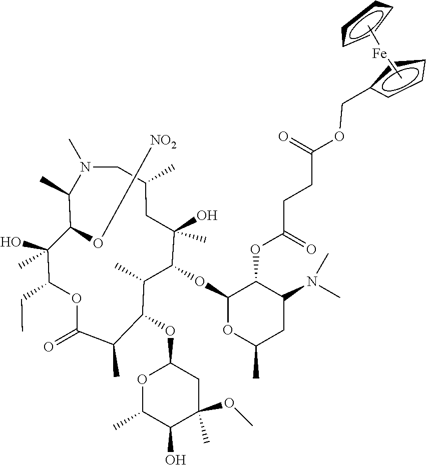

Compound E-10 (200 mg, 0.25 mmol) was dissolved in dry dichloromethane (5 mL). To this was added subsequently, 4-dimethylaminopyridine (4-DMAP, 3 mg, 0.25 mmol. 0.1 eq.) and succinic anhydride (28 mg, 0.28 mmol). The reaction was stirred overnight at room temperature. The solvent was removed in vacuo and the resulting white amorphous foam was used directly for the next step.

Fresh dry dichloromethane (5 mL) was added to the resulting foam, followed by 1-Hydroxy-methylferrocene (60 mg, 0.28 mmol, 1.1 eq.). The reaction was cooled to 0° C. and to this was added EDCI (96 mg, 0.5 mmol, 2 eq.). The reaction was allowed to progressively warm to room temperature where it was stirred overnight. Additional dichloromethane (20 mL) was added and washed several times with saturated aqueous ammonium chloride, brine (2×), dried under anhydrous Na₂SO₄ and the solvent removed in vacuo. The resulting crude product was purified by chromatography with a gradient starting at 10% of acetone in cyclohexane (0.2% Et₃N) yields 150 mg of the desired product (53%).

Similarly, the following compound may be obtained using the procedure above starting from compound E-19.

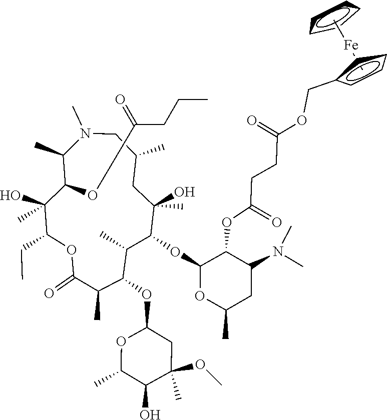

### Example 34

Activity of substances in the inhibition of growth of bacteria. Bacteria including the species Escherichia coli, Bacillus pumilus, Salmonella sp., Micrococcus luteus and Staphylococcus carnosus are cultured in appropriate media (Luria broth for all except S. canosus). Overnight cultures are mixed with fresh medium to reach an optical density at 600 nM of ca. 0.1 AU. These cultures are mixed with solutions of substances to be tested at concentrations ranging from 100 μM to 0.05 μM in a microtitre plate. The growth of the culture is monitored by measuring the optical density at various times after the addition of the inhibitor. Reduction in the rate of increase in optical density corresponds to an inhibition of bacterial growth. In the following tables, the activity of various of the test substances may be observed by reductions in optical density relative to untreated control cultures. The data are summarized in Table 3 and Table 4.

TABLE 12
Inhibition of growth of Staphylococcus carnosus
by compounds after 9-20 h:

| Conc. | Absorbance of culture medium at 600 nm | | | | | | | |
|---|---|---|---|---|---|---|---|---|
| (μM): | 100 | 50 | 25 | 13 | 6 | 3 | 2 | 0.8 |
| E-2 | 0.395 | 0.425 | 0.456 | 0.542 | 0.626 | 0815 | 0.766 | 0.760 |
| E-5 | 0.302 | 0.347 | 0.403 | 0.452 | 0.525 | 0.805 | 0.726 | 0.819 |
| E-13 | 0.421 | 0.437 | 0.282 | 0.488 | 0.530 | 0.668 | 0.755 | 0.765 |
| E-1 | 0.157 | 0.096 | 0.078 | 0.068 | 0.625 | 0.864 | 0.856 | 0.902 |
| E-12 | 0.494 | 0.523 | 0.574 | 0.548 | 0.591 | 0.577 | 0.688 | 0.783 |
| E-9 | 0.545 | 0.522 | 0.426 | 0.677 | 0.752 | 0.830 | 0.737 | 0.765 |
| E-10 | 0.576 | 0.433 | 0.595 | 0.702 | 0.699 | 0.768 | 0.826 | 0.862 |
| E-11 | 0.641 | 0.574 | 0.819 | 0.822 | 0.887 | 0.890 | 0.918 | 0.941 |

| | |
|---|---|
| No | 0.887 |
| compound | |

TABLE 13
Inhibition of growth of Salmonella typhimurium
by compounds after 9-20 h:

| Conc. | Absorbance of culture medium at 600 nm | | | | | | | |
|---|---|---|---|---|---|---|---|---|
| (μM): | 100 | 50 | 25 | 13 | 6 | 3 | 2 | 0.8 |
| E-2 | 0.251 | 0.168 | 0.314 | 0.621 | 0.382 | 0.410 | 0.441 | 0.452 |
| E-5 | 0.390 | 0.395 | 0.396 | 0.437 | 0.478 | 0.511 | 0.568 | 0.574 |
| E-13 | 0.398 | 0.407 | 0.427 | 0.459 | 0.505 | 0.543 | 0.605 | 0.634 |
| E-1 | 0.557 | 0.484 | 0.736 | 0.722 | 0.741 | 0.711 | 0.761 | 0.722 |
| E-12 | 0.332 | 0.270 | 0.326 | 0.343 | 0.400 | 0.354 | 0.457 | 0.455 |
| E-9 | 0.221 | 0.319 | 0.395 | 0.382 | 0.348 | 0.327 | 0.272 | 0.300 |
| E-10 | 0.280 | 0.315 | 0.390 | 0.399 | 0.382 | 0.402 | 0.361 | 0.334 |
| E-11 | 0.562 | 0.650 | 0.697 | 0.633 | 0.627 | 0.623 | 0.587 | 0.555 |

| | |
|---|---|
| No | 0.943 |
| compound | |

### Example 35

Substances may act directly on bacteria, or they may act to promote the killing of the bacteria by phagocytes. To measure this effect, cultures murine macrophages are incubated with a test bacteria and the number of bacteria surviving are counted in terms of the viable colony forming units (CFU). The method for determining the rate of phagocytosis is as follows:

Intracellular killing of S. Typhimurium by mouse macrophage cell line J 774 A.1
seed a monolayer of cells in 200 μl Media into the wells of a 96 well plates
incubate O/N 37° C.
remove medium and add fresh medium
add Bacteria (Salmonella typhimurium) e.g. 5 μl of 1:100 diluted O/N culture (MOI=10) (=108 cfu/ml)
centrifuge 10 min 800 g (—2000 rpm)
incubate 20-30 minutes at 37° C. (phagocytosis)
remove media
wash 1-2× with PBS
add medium with 100 μg/ml Gentamicin, stock: 10 mg/ml (=1:100)
incubate 45′ at 37° C.
wash 2× with PBS
add fresh medium (200 μl/well)
add compounds to test
incubate 2-3 hours at 37° C.
remove medium
lyse cells with water: add 200 μl H20 incubate 10′, push a few times through 27 gauge needle using a 1 ml syringe
plate 100 μlf 1:10 dilution onto LB-agar plates (=1:100 dil)

Monolayer of J 774 A.1 in 96 well plate=˜1-5×104 cells

Overnight culture of Salmonella thyph.=˜1×1010 cfu/ml

Overnight culture of Staph. carnosus=˜5×109 cfu/ml MOI=50 (=5 μl of 1:10 dil. O/N culture)

MOI=Multiplicity of Infection

Medium: DMEM/RPMI 7.5% FCS

### Example 36

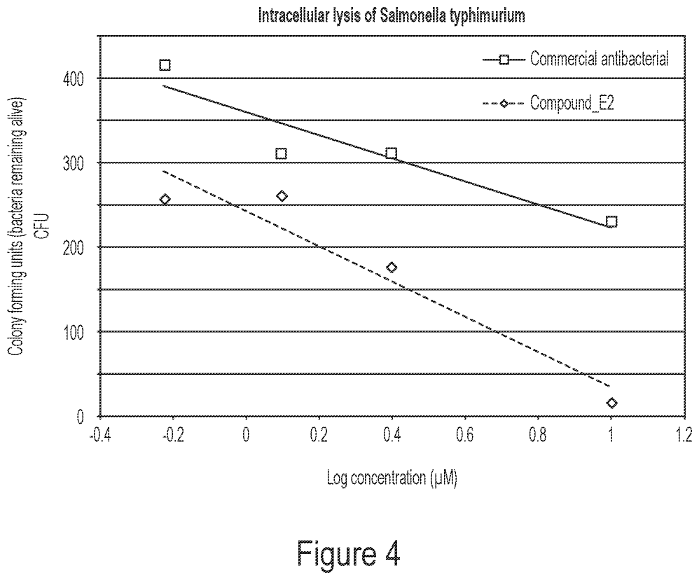

The potential efficacy of a Compound for Inflammatory bowel disease may be modeled as follows. C57 BLK6 or BALBc mice are provided with drinking water containing 2.5% or 2.8% dextran sulfate. Animals are weighed and observed for signs of intestinal disturbance daily. Signs include diarrhea or occult blood. Compound is formulated by mixing with a solution of 0.1 up to 1% citric acid depending on concentration. Compound is provided by oral gavage daily. Example data for the efficacy of compounds cited here is provided in FIG. 3, 7, 8 or 10-19.

### Example 37

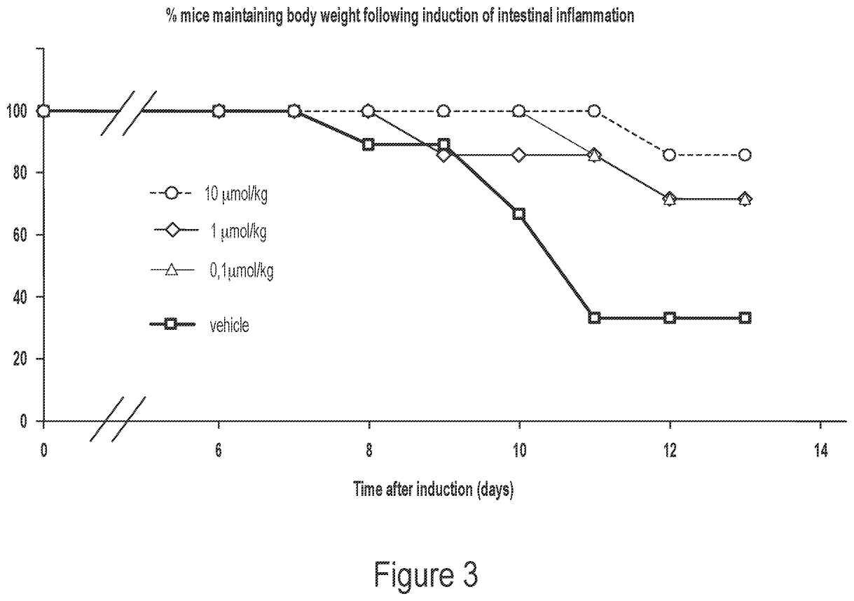

The potential efficacy of a Compound for rheumatoid arthritis may be modeled as follows. DBA1 mice are induced by a subcutaneous injection of bovine collagen in 0.05M acetic acid, emulsified in Freund's adjuvant. 21 days later, a second injection of this material is made without inclusion of mycobacterial material in the adjuvant. Animals are weighed and observed for signs of inflammation daily. Signs include weight loss, swelling of paws, redness and reduced mobility. Compound is formulated by mixing with a solution of 1% citric acid. Compound is provided by oral gavage daily. Data for the efficacy of compounds cited here is provided in FIG. 2.

### Example 38

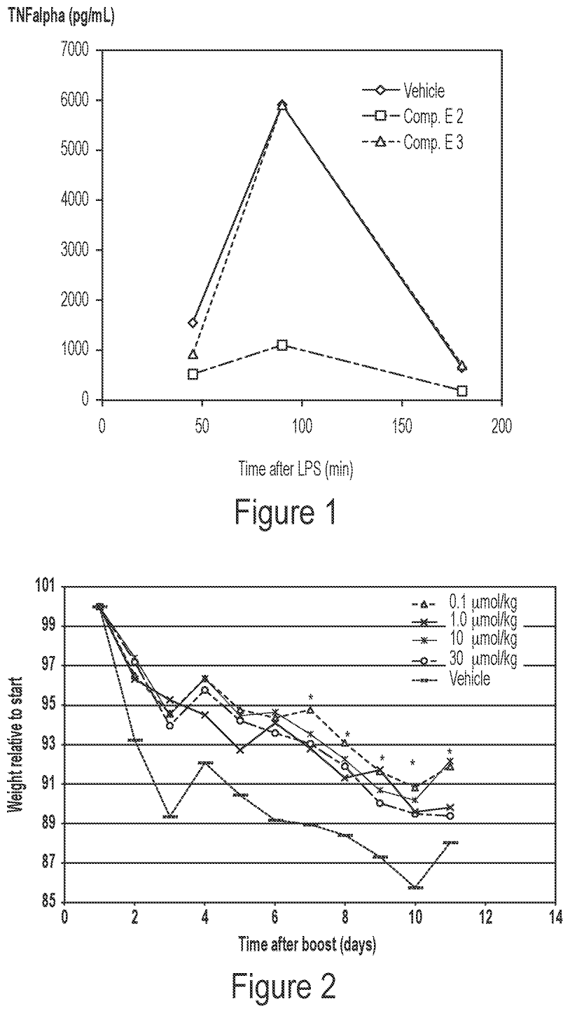

The potential efficacy of a Compound in modulating immune reactions may be determined as follows. Swiss or C57 Blk6 mice are induced to produce cytokines by a subcutaneous injection of lipopolysaccharide. Typically, compound is provided at time 0. Compound is formulated by mixing with a solution of 1% citric acid for oral treatment or, dissolved in PEG 300 and diluted in water for intra-peritoneal treatment. Compound is provided by oral gavage. 30 minutes after providing compound, animals are treated with an intra-peritoneal injection of a solution of lipopolysaccharide in the concentration range that will provide 0.01 mg/kg lipopolysaccharide. Data for the efficacy of compounds cited here is provided in FIG. 1.

### Example 39

The potential efficacy of a Compound in treating a malignant disease may be determined as follows. Tumours are known to be deficient in nitric oxide and this is considered to be a cause of local tolerance. Providing a nitric oxide donor that is accumulated in macrophages in the tumour environment provides a means to artificially modify the local NO status. C57 Blk6 mice are injected subcutaneously with an murine ovarian cancer cell line expressing ovalbumin. Mice bearing tumours are selected after 14 days. Typically, compound is provided at this time. Compound is formulated by mixing with a solution of 1% citric acid for oral treatment. Compound is provided by oral gavage. Animals are monitored daily for tumour size, body weight and activity score. The activity of the compound may be determined in combination with other therapies including anti-bodies or vaccines based on a tumour antigen. In this case ovalbumin, can serve as a model antigen.

### Example 40

### Synthesis of 2′-O-(2-Ferrocenyl) acetyl-11-O-nitro-azithromycin

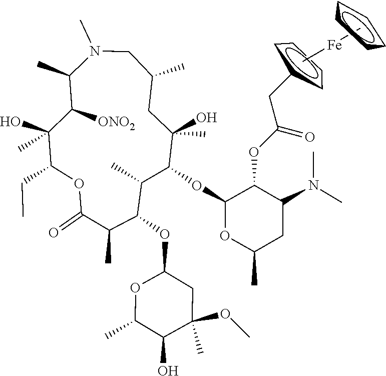

11-O-Nitro-azithromycin (0.25 mmol) was dissolved in dry dichloromethane (5 mL). To this was added EDCI (2 eq., 0.5 mmol) and 2-ferrocenyl acetic acid (1.1 eq., 0.28 mmol). The reaction was stirred overnight at room temperature. The solvent was removed in vacuo and the resulting white amorphous foam. The resulting crude product was purified by column chromatography with a gradient starting at 10% of acetone in cyclohexane (0.2% Et₃N).

Similarly, the following compound may be obtained using the procedure above starting from 2′-O-Nitro-azithromycin:

### 2′-O-Nitro-11-O-(2-ferrocenyl) acetyl-azithromycin

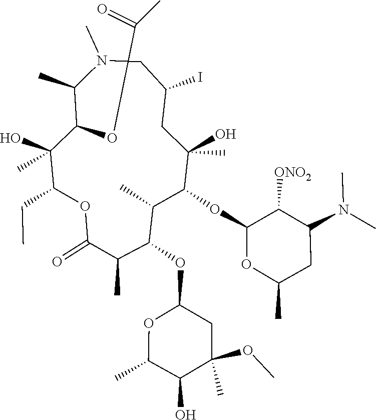

### Example 41. rac. 2′-O-Propionyl Propranolol

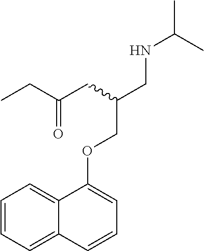 [SMILES: CCC(=O)CC(CNC(C)C)COc1cccc2ccccc12]

Diethyl azodicarboxylat (DEAD, 10 mmol) is added to a stirred (magnetic stirrer, 300 rpm) solution of triphenylphosphin (10 mmol) in dry THF (25 mL) at 0° C. and treatment is continued for 30 min. Propranolol (5 mmol) and propionic acid (10 mmol) both dissolved in THF (10 mL) are added dropwise and stirring is continued for 1 h at 0° C. and further 2 hours at ambient temperature. Any precipitates are filtered off, the remaining solution is concentrated in vacuo and desired product is isolated by column chromatography (silica gel, cyclohexane—ethyl acetate).

### Example 44. rac. 2′-O-Acetoxypropionyl Propranolol

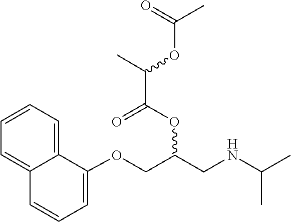 [SMILES: CC(=O)OC(C)C(=O)OC(CNC(C)C)COc1cccc2ccccc12]

Diethyl azodicarboxylate (DEAD, 10 mmol) is added to a stirred (magnetic stirrer, 300 rpm) solution of triphenylphosphin (10 mmol) in dry THF (25 mL) at 0° C. and treatment is continued for 30 min. Propranolol (5 mmol) and 2-acetoxypropionic acid (10 mmol) both dissolved in THF (10 mL) are added dropwise and stirring is continued for 1 h at 0° C. and further 2 hours at ambient temperature. Any precipitates are filtered off, the remaining solution is concentrated in vacuo and desired product is isolated by column chromatography (silica gel, cyclohexane—ethyl acetate).

### Example 43 Synthesis of azithromycin 11,2′-dilipoate

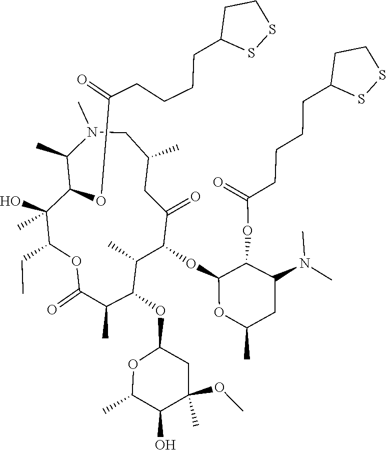

380 mg of azithromycin-2′-lipoate (E-77) are dissolved in 25 ml of dichloromethane and cooled in an ice bath. 125 mg of lipoyl are added, then 50 μl of pyridine. The mixture is allowed to reach room temperature and stirred for 16 h. The reaction mixture is extracted with water 3 times, then once with 5% aqueous citric acid. The citric acid phase is extracted with dichloromethane, then combined with ethyl acetate and carefully made basic with sodium hydrogen carbonate and vigorous stirring. When gas evolution ceases, the organic phase is separated, washed with water and brine, and dried with sodium sulfate. After evaporation of all volatiles, the residue is chromatographed with a gradient starting at cyclohexane-acetone 5-1, containing 0.5% of triethylamine. Yield: 140 mg

### Example 44 Synthesis of Azithromycin 11-lipoate

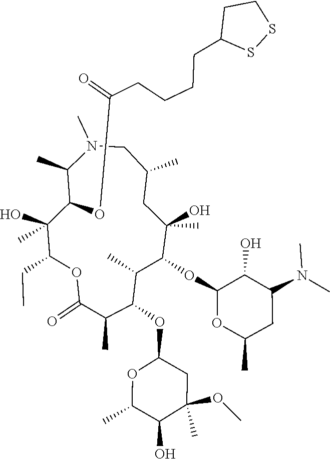

350 mg of Azithromycin 11,2′-dilipoate are stirred with 5 ml methanol at room temperature. When mass spectrometry indicates completion of the reaction (m/z=1125.5->937.5), the mixture is partitioned between water and ethyl acetate. The organic phase is washed once with water, then extracted with 5% aqueous citric acid. The citric acid phase is extracted with dichloromethane, then combined with ethyl acetate and carefully made basic with sodium hydrogen carbonate and vigorous stirring. When gas evolution ceases, the organic phase is separated, washed with water and brine, and dried with sodium sulfate. After evaporation of all volatiles, the residue is chromatographed with a gradient starting at cyclohexane-acetone 5-1, containing 0.5% of triethylamine. Yield: 225 mg

### Example 45. Formation of Acetic Esters of ALCs

Method 1: ALC (1.0 mmol) was taken up in 15 mL dichloromethane. Pyridine (1.2 eq.) was added and the resulting solution was cooled in an ice bath for approximately 10 minutes. At this point, a solution of acetic anhydride (1.2 eq) was added dropwise. The reaction was stirred continually at this temperature and then progressively warmed to room temperature where it was stirred overnight. Reaction progress was monitored either by TLC and/or MS. The reaction was washed with a saturated solution of ammonium chloride (3×), water (3×) and dried over anhydrous Na₂SO₄. The solvent was evaporated in vacuo. Co-evaporation with toluene is necessary to remove residual pyridine from the system. This was followed by re-dissolving the residue in DCM and solvent evaporation twice to produce a white foam, which was dried under high-vacuum to produce acetylated product.

This acetylation conditions can be extended for other ALCs. In the case where the acetylation proceeds sluggish, alternative reaction conditions were undertaken as described below:

Method 2. Compound A-12 (0.85 mmol) was taken up in DCM (10 mL). To this was added triethylamine (3.5 eq) and acetyl chloride (3.5 eq). Reaction was monitored by TLC and MS until disappearance of starting ALC. Reaction was filtered. The filtrate was either evaporated in vacuo and directly purified by column chromatography or the filtrate was washed with 10% aq. Na₂CO₃ solution, brine, dried over Na₂SO₄ and evaporated in vacuo to get the crude product.

Method 3. Acetic acid (4 eq) was taken up in 5 mL dichloromethane (DCM). Compound A-16 (0.5 mmol) and 4-dimethylaminopyridine (DMAP) (4.4 eq) were added and the resulting solution was cooled in an ice bath for approximately 10 minutes. At this point, dicyclohexylcarbodiimide (DCC) (4.4 eq) was added slowly. The reaction was stirred continually at this temperature for 5 minutes and then progressively warmed to room temperature where it was stirred overnight. Dicyclohexylurea (DCU) that was formed during the reaction is filtered off and discarded. The filtrate was collected and then washed with a saturated solution of sodium hydrogencarbonate (3×), water (1×) and dried over anhydrous Na₂SO₄. The solvent was evaporated in vacuo. This was followed by re-dissolving the residue in a small volume of methanol. The solution was transported dropwise into ice-cold water (2× volume of methanol) and stored in the freezer overnight. The precipitated product was filtered off and dried under high-vacuum to produce a product.

TABLE 14
Acetylation Examples

| | | | | | | | MS |
|---|---|---|---|---|---|---|---|
| Compound | Synthesis | | Substituent | Reaction Condition | Degree of | | m/z |
| Entry | Method | ALC | equivalent | (i.e. Workup) | Substitution | Yield | ([M + H]⁺) |
| E-2 | 1 | A-1 | 1.2 | as described above | 1 | 76% | 837 |
| E-4 | 1 | A-1 | 1.5 | as described above | 1 | 41% | |
| E-8 | 1 | A-1 | 1.2 | | | 39% | |
| E-23 | 1 | A-1 | 2.0 | as described above | 2 | 54% | |
| E-25 | 1 | A-1 | 1.1 | as described above | 1 | 67% | |
| E-418 | 3 | A-16 | 4 | as described above | 3 | 20% | CHMA02063 |
| E-228 | 1 | A-10 | 4 | as described above | 2 | 56% | 673 |
| E-453 | A-17 | Overall | | 1 and 2 | 49% | 819.7 | A-17 |
| | | 12 equiv. | | | (referring to | 861.5 | |
| | | Ac₂O | | | the di-ester) | | |
| E-266/E-268 | 2 | A-12 | 3 | as described above, | 2 > 4 | 77% | 675, 759 |
| | | | | filtered through a silica | | | |
| | | | | gel plug using | | | |
| | | | | CHCl₃:iPrOH:7M NH₃ in | | | |
| | | | | MeOH (30:1:1) as mobile | | | |
| | | | | phase | | | |
| E-23/E-50 | | A-1 | | | 2 and 3 | 16% | 833, 875 |
| E-564 | 1 | A-2 (X = O) | | | 1 | 89% | 776 |
| E-29 | 1 | E-19 | 1.2 | direct chromatography | 1x | 85% | 861 |
| | | | | for purification | | | |

### Example 46. Formation of Butyric and Isobutyric Esters of ALCs

Method 1: Compound A-1 was taken up in dichloromethane and stirred for 10 min. At this point, a solution of carboxylic anhydride and triethylamine in dichloromethane was added dropwise. The reaction was stirred continually at room temperature. The reaction solution was washed with 5% citric acid three times to extract the product. Acidic solution was then washed with ethyl acetate (2×) and afterwards neutralized with Na₂CO₃. Product was extracted with ethyl acetate (3×). The solution was washed with a saturated solution of sodium chloride (2×), water (2×) and dried over anhydrous Na₂SO₄. The solvent was evaporated in vacuo to produce a white foam containing product.

Method 2: Compound A-1 was taken up in dichloromethane and was cooled in an ice bath for approximately 10 minutes. At this point, a solution of carboxylic chloride in dichloromethane was added dropwise. The reaction was stirred continually at this temperature for 15 min and then progressively warmed to room temperature where it was stirred for 2.5 h.

The reaction was washed with a 10% solution of Na₂CO₃ (3×), water (3×) and dried over anhydrous Na₂SO₄. The solvent was evaporated in vacuo. Co-evaporation with toluene is necessary three times. This was followed by re-dissolving the residue in dichloromethane to produce a white foam, which was dried under high-vacuum to produce product.

Method 3: Starting material was taken up in dichloromethane and stirred for 10 min. At this point, a solution of carboxylic chloride and triethylamine in dichloromethane was added dropwise. The reaction was stirred continually at room temperature for two days. The reaction solution was washed with 5% citric acid three times to extract the product. Acidic solution was then washed with ethyl acetate (2×) and afterwards neutralized with Na₂CO₃. Product was extracted with ethyl acetate (3×). The solution was washed with a saturated solution of sodium chloride (2×), water (2×) and dried over anhydrous Na₂SO₄. The solvent was evaporated in vacuo to produce a white foam containing product.

Method 4: Compound E-48 or E-39 was solved in methanol to hydrolyze butyric esters. The reaction was stirred continually at room temperature for two days. The reaction solution was washed with ethyl acetate three times to extract the product. The ethyl acetate phase was washed with 5% citric acid (3×). Acidic solution was then washed with ethyl acetate (2×) and afterwards neutralized with Na₂CO₃. Product was extracted with ethyl acetate (3×). The solution was washed with a saturated solution of sodium chloride (2×), water (2×) and dried over anhydrous Na₂SO₄. The solvent was evaporated in vacuo to produce a white foam containing product.

Method 5: Carboxylic acid (180 mg, 2.04 mmol) was solved in 3 mL dichloromethane. Under stirring conditions 4-Dimethylaminopyridine (274 mg, 2.24 mmol) and A-16 were added. The reaction solution was cooled to 0° C. and N,N′-Dicyclohexylcarbodiimide (463 mg, 2.24 mmol) was added. The reaction was stirred continually at this temperature for 5 min and then progressively warmed to room temperature where it was stirred for 12 h. Precipitation was removed via filtration. The reaction was washed with a saturated solution of NaHCO₃ (3×) and dried over anhydrous Na₂SO₄. The solvent was evaporated in vacuo. Product was solved in methanol and water was added. Solution was cooled to −20° C., a precipitation occurred which was extracted and dried in vacuo.

Workup 1: Column chromatography over silica gel was carried out to separate different products. As eluent a mixture of chloroform, 2-propanol and ammonia in methanol (60:1:1) was used. The solvent was evaporated in vacuo.

Workup 2: Preparative chromatography over RP-C18-silica gel was carried out to separate different products. As eluent a mixture of water (with trifluoroacetic acid 0.05%) and methanol (with trifluoroacetic acid 0.05%) was used. The solvent was evaporated in vacuo.

Workup 3: Column chromatography over silica gel was carried out to separate different products. As eluent a mixture of cyclohexane, acetone (7:1) with 0.5% triethylamine was used. The solvent was evaporated in vacuo.

TABLE 15
Butyrylation/Isobutyrylation Examples

| Compound | Synthesis | | | Reaction Condition | Degree of | | |
|---|---|---|---|---|---|---|---|
| Entry | Method | ALC | Substituent | (i.e. Workup) | Substitution | Yield | MS |
| E-35 | 1 | A-1 | Butyric | Workup 1 | 1x | 76% | 819 |
| E-39 | 3 | A-1 | Butyric | Workup 2 | 2x | 10% | 889 |
| E-48 | 3 | A-1 | Butyric | Workup 2 | 3x | 12% | 959 |
| E-47 | 3 | A-1 | Butyric | Workup 2 | 4x | 10% | 1029 |
| E-424 | 5 | A-16 | Butyric | none | 3x | 97% | 944 |
| E-458 | 2 | A-17 | Butyric | none | 1x | 89% | 847 |
| E-19 | 4 | E-39 | Butyric | Workup 2 | 1x | 85% | 819 |
| E-82 | 4 | E-48 | Butyric | Workup 3 | 1x | 35% | 819 |
| E-553 | 4 | E-48 | Butyric | Workup 3 | 2x | 30% | 889 |
| E-44 | 1 | A-1 | Isobutyric | Workup 1 | 1x | 67% | 819 |
| E-458, E-459, E-460 | 3 | A-17 | Butyric | Workup 1 | 1x, 2x, 3x | 86% | 847, 917, 987 |
| E-238 | 1 | A-10 | Butyric | none | 1x | 96% | 659 |
| E-241, E-249, E-250 | 3 | A-10 | Butyric | Workup 1 | 2x, 3x, 4x | 85% | 730, 800, 870 |
| E-111 | 3 | E-1 | Butyric | Workup 2 | 1x | 38% | 864 |
| E-255* | 1 | A-11 | Butyric | Workup 1 | 2x | 3% | 892 |
| E-256* | 1 | A-11 | Butyric | Workup 1 | 3x | 5.6% | 964 |
| E-85* | 1 | A-11 | Butyric | Workup 1 | 3x | | 964 |
| E-257* | 1 | A-11 | Butyric | Workup 1 | 4x | 3% | 1036 |
| E-24 | 3 | E-1 | Butyric | Workup 1 | 1x | 75 | 864 |
| E-89 | 1 | A-3 | Butyric | n/a | 1x | 9% | 406 |

*isolated from one reaction

### Example 47. Formation of Valeric Esters of ALCs

Method 1. The ALC was taken up in DCM. To this were added pyridine and valeric acid anhydride (1 equiv. pyridine/1 equiv. valeric acid anhydride). The mixture was stirred at room overnight or over the weekend and was then poured on an aqueous citric acid solution (5% or 10%) at RT and was stirred for 15 min. The aqueous phase was extracted with EtOAc (2×) and was afterwards brought to pH=8 with solid Na₂CO₃. The alkaline aqueous layer was extracted with EtOAc (2×) and the combined organic phases were washed with water (1×) and saturated aqueous NaCl-solution (1×), dried (Na₂SO₄), concentrated to dryness and dried at the oil pump. Products were obtained as colorless solids or foams.

Method 2. Analogue to 2A but after stirring at RT for 2 h additional pyridine (2 equiv.) and valeric acid anhydride (2 equiv.) were added and stirring was continued overnight. Products were obtained as colorless foams or solids.

Method 3. The ALC was taken up in DCM. To this were added pyridine (4 equiv.) and valeric acid anhydride (4 equiv.). The mixture was stirred at room overnight or over the weekend. Additional pyridine (2 equiv.) and valeric acid anhydride (2 equiv.) were added and the mixture was stirred overnight. Reaction mixture was filled into a separation funnel and washed with saturated aqueous NH₄Cl-solution (3×) and water (3×). The organic phase was dried (Na₂SO₄) and concentrated to dryness. The residue was co-evaporated with toluene

(3×) and with DCM (3×). Afterwards the crude product was purified by column chromatography on silica gel. Eluent:Chloroform/Isopropanol/NH₃ (7 M in Methanol) 30/1/1

The product was dried at the oil pump. Products were obtained as colorless solids or foams.

TABLE 16
Valeric Ester Examples

| | | | Overall | | | | |
|---|---|---|---|---|---|---|---|
| | | | equiv. of | | | | MS |
| Compound | Synthesis | | acid or | | Degree of | | m/z |
| Entry | Method | ALC | anhydride | Re | Substitution | Yield | ([M + H]⁺) |
| E-72 | 3 | A-1 | 6 | Amount DCM: 5 mL | 2 | 65% | 917.5 |
| E-558 | 1 | A2 | 5 | Anhydro erythromycin | 1 | 7% | 800.5 |
| | | (Anhydro) | | products are formed | | | |
| | | | | Amount DCM: 25 mL | | | |
| E-569 | 3 | A-10 | 6 | Amount DCM: 5 mL | 2 | 45% | 757.5 |
| E-582 | 2 | A-12 | 6 | Amount DCM: 10 mL | 1 | 89% | 675.5 |
| | | | | Citric acid: 10% | | | |
| E-411 | 1 | A-15 | 4 | Amount DCM; 25 mL | 1 and 2 | 81% | 878.3 |
| E-412 | | | | Citric acid:5% | | | 962.3 |
| E 428 | 1 | A-16 | 3 | Amount DCM: 25 mL | 1 and 2 | 83% | 819.0 |
| E-429 | | | | Citric acid: 5% | | | 902.8 |
| E-464 | 1 | A17 | 5 | Amount DCM: 25 mL | 1 | 94% | 861.7 |
| | | | | Citric acid: 5% | | | |

### Example 48. Formation of Isovaleric esters of ALCs

Method 1: Isovaleric acid (4.4 equiv./equiv. ALC) and HOBt 85% (4.4 equiv./equiv. ALC) were dissolved in DMF (12.5 mml/mmol ALC). The solution was cooled down to 0-5° C. in an ice-bath. At this temperature a solution of Dicyclohexylcarbodiimide (4.5 equiv./equiv. ALC) in DCM (5 ml/mmol ALC) was added dropwise within 30 min. the solution was kept at this temperature for another 10 min. Then Azithromycin (1 equiv.) was added in one portion. While stirring, the solution was allowed to come to room temperature within 2 h. Stirring was continued for another 2 h at 50° C. The reaction mixture was allowed to stand at RT for 12 h. A white precipitate was removed by suction. The solvent was evaporated completely at 12 mbar and 50° C. The residue was dissolved in DCM (12.5 mL/mmol ALC) and washed with water (7.5/mmol ALC). A small amount of a white precipitate was removed. Then the solution was treated with citric acid (25 mL/mmol ALC, 5%). The aqueous phase was washed with DCM (5 ml/mmol ALC). NaOH 10% was added until the aqueous phase was basic (pH 12) and was washed with DCM (2×10 ml/mmol ALC). After phase separation the organic phase was evaporated to dryness, products were obtained as white solids.

Method 2A. The ALC was taken up in DCM. To this were added pyridine and isovaleric acid anhydride (1 equiv. pyridine/1 equiv. isovaleric acid anhydride). The mixture was stirred at room overnight or over the weekend and was then poured on an aqueous citric acid solution (5% or 10%) at RT and was stirred for 15 min. The aqueous phase was extracted with EtOAc (2×) and was afterwards brought to pH=8 with solid Na₂CO₃. The alkaline aqueous layer was extracted with EtOAc (2×) and the combined organic phases were washed with water (1×) and saturated aqueous NaCl-solution (1×), dried (Na₂SO₄), concentrated to dryness and dried at the oil pump. Products were obtained as colorless solids or foams.

Method 2B. Analogue to 2A but after stirring at room temperature for 2 h additional pyridine (2 equiv.) and isovaleric acid anhydride (2 equiv.) were added and stirring was continued overnight.

Products were obtained as colorless foams or solids.

Method 2C. The ALC was taken up in DCM. To this were added pyridine (4 equiv.) and isovaleric acid anhydride (4 equiv.). The mixture was stirred at room for approximately 2 h, then a catalytic amount of DMAP was added, followed by another catalytic amount approximately another 2 h later. The mixture was stirred at room temperature for approximately 2 h before additional pyridine (2 equiv.) and isovaleric acid anhydride (2 equiv.) were added. The mixture was stirred at room overnight and then poured on an aqueous citric acid solution (5%,) and stirred at room temperature for 30 min. The aqueous phase was extracted with EtOAc and afterwards brought to pH=8 with solid Na₂CO₃. The alkaline aqueous layer was extracted with EtOAc (2×) and the combined organic phases were washed with water (1×) and saturated aqueous NaCl-solution (1×), dried (Na₂SO₄), concentrated to dryness and dried at the oil pump. Products were obtained as colorless solids or foams.

TABLE 17
Isovaleric Ester Examples

| | | | Overall | | | | |
|---|---|---|---|---|---|---|---|
| | | | equiv. of | | | | MS |
| Compound | Synthesis | | acid or | | Degree of | | m/z |
| Entry | Method | ALC | anhydride | Annotations | Substitution | Yield | ([M + H]⁺) |
| E-45 | 1 | A-1 | | | 1 | 91% | 833.5 |
| E-557 | 2A | A2 | 5 | Anhydro erythromycin | 1 | 17% | 800.5 |
| | | (Anhydro) | | products are formed | | | |
| | | | | Amount DCM: 25 mL | | | |
| | | | | Citric acid: 5% | | | |
| E-573 | 2C | A-10 | 6 | Amount DCM: 7.5 mL | 2 | 19% | 757.5 |
| | | | | Citric acid: 5% | | | |
| E-583 | 2B | A12 | 6 | Amount DCM: 10 mL | 1 | 83% | 675.5 |
| | | | | Citric acid: 10% | | | |
| E-413 | 2A | A-15 | 4 | Amount DCM: 25 mL | 1 | 95% | 878.3 |
| | | | | Citric acid: 5% | | | |
| E 431 | 2A | A-16 | 3 | Amount DCM: 25 mL | 2 | 87% | 818.8 |
| E-432 | | | | Citric acid: 5% | | | 902.7 |
| E-467 | 2A | A-17 | 5 | Amount DCM: 25 mL | 1 | 90% | 861.6 |
| | | | | Citric acid: 5% | | | |

### Example 49. Long Chain (>C5) Fatty Acid Substation of Tildipirosin

Hexanoic acid (290 mg, 2.5 mmol) was taken up in 5 mL dichloromethane (DCM). Compound A-16 (367 mg, 0.5 mmol) and 4-Dimethylaminopyridine (DMAP) (336 mg, 2.75 mmol) were added and the resulting solution was cooled in an ice bath for approximately 10 minutes. At this point, dicyclohexylcarbodiimide (DCC) (567 mg, 2.75 mmol) was added slowly. The reaction was stirred continually at this temperature for 5 minutes and then progressively warmed to room temperature where it was stirred overnight. Dicyclohexylurea (DCU) that was formed during the reaction is filtered off and discarded. The filtrate was collected and then washed with a saturated solution of ammonium chloride (3×), sodium hydrogencarbonate (3×), water (lx) and dried over anhydrous Na₂SO₄. The solvent was evaporated in vacuo. This was followed by re-dissolving the residue in a small volume of methanol. The solution was transported dropwise into ice-cold water (2× volume of methanol) and stored in the freezer overnight. The precipitated product was filtered off and dried under high-vacuum to produce a mixture of E-437, E-438 and E-439 (56%).

This esterification conditions can be extended for other acids.

TABLE 18
Long chain fatty acid substitution of ALC

| | | | Degree of | | |
|---|---|---|---|---|---|
| Compound | | Substituent | Substitu- | | |
| Entry | Acid | equivalent | tion | Yield | MS |
| E-437, | Hexanoic acid | 5 | 2 and 3 | 56% | 931.7 |
| E-438, | | | | | 1028.8 |
| E-439 | | | | | |
| E-440, | Heptanoic acid | 5 | 1, 2 and 3 | 46% | 846.9 |
| E-441, | | | | | 959.3 |
| E-442 | | | | | 1071.9 |
| E-445 | Octanoic acid | 5 | 3 | 17% | 1113.5 |
| E-447, | Decanoic acid | 5 | 2 and 3 | 41% | 1043.3 |
| E-448 | | | | | 1197.5 |
| E-450, | Dodecanoic | 5 | 2 and 3 | 60% | 1099.5 |
| E-451 | acid | | | | 1281.4 |

### Example 50. General Procedure for Preparing Cores A-13 and A-14

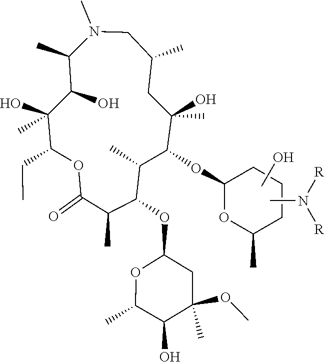

Macrolide (1 mmol) is dissolved in DMF (500 l). Epichlorohydrin (1 mL) is added and the mixture is heated to 80° C. for 12 h. When MS analysis indicates complete conversion, all volatiles are removed in vacuo and the residue is dissolved in ethanol (1 ml). The solution is poured into 25 ml of water. The precipitate is isolated and can be used directly for the next step or is chromatographed to obtain the pure epoxide.

The following macrolides were used to form epoxides as precursor to the desired cores.

TABLE 19
Epoxide Formation

| | | MW | |
|---|---|---|---|
| Entry | Macrolide | epoxide | Yield |
| 1 | Azithromycin | 703 | 25% |
| 2 | Gamithromycin | 731 | 12% |
| 3 | 3-decladinosyl-3-oxoazithromycin | 543 | 5% |
| 4 | Tildipirosin | 689 | 5% |

Epoxide (1 mmol) is dissolved in 2-propanol (500 μl), and an excess of 5 equivalent of an amine is added. The mixture is heated from 12 h to 100 h at 80° C. When MS indicates complete conversion, all volatiles are evaporated and the residue subjected to chromatography to separate the 2 regioisomeric amines.

TABLE 20
Epoxide opening by amines

| | | Epoxide | | | | | |
|---|---|---|---|---|---|---|---|
| Entry | | opening | | | | | |
| (Table 9) | Amine | position | R¹ | R² | [M + H]⁺ | Yield [%] | Comment |
| 1 | dimethylamine | 3′ | Me | Me | 749 | 66 | NMR identical |
| | | | | | | | to azithromycin |
| | | 2′ | | | | 22 | regioisomer of |
| | | | | | | | Azithromycin |
| 1 | morpholine | 3′ | R² = R² = morpholine ring | | 791 | 30 | unpolar product |
| | | (A-13.1) | | | | | |
| | | 2′ | | | | 61 | polar product |
| | | (A-13.2) | | | | | |
| 1 | diethanolamine | 3′ | C₂H₄OH | C2H4OH | 809 | 22 | unpolar product |
| | | (A-14.1) | | | | | |
| | | 2′ | | | | 47 | polar product |
| | | (A-14.2) | | | | | |
| 1 | ammonia | | H | H | 721 | n.d.* | |
| 1 | N-methyl | | Me | OH | 751 | n.d.* | |
| | hydroxylamine | | | | | | |
| 1 | Iminodiacetic | | —CH₂C(O)OEt | —CH₂C(O)OEt | 893 | n.d.* | some cyclized |
| | acid | | | | | | product with |
| | diethylester | | | | | | [M + H]⁺ = 865 is |
| | | | | | | | formed, too |

| | | | |
|---|---|---|---|
| 2 | morpholine | R² = R² = morpholine ring | n.d.* |

*n.d. not determined

### Example 51. Further Acylation Reactions of ALC

Method 1: Compound A-1 (2000 mg, 2.67 mmol) was taken up in 10 mL dichloromethane and stirred. Separately 4.4 eq of a carboxylic acid and 4.4 eq of 1,1′-Carbonyldiimidazole were solved in dichloromethane (10 mL) and stirred over 20 min. Both solutions were unified and stirred continually at room temperature. The dichloromethane phase was washed with saturated NaHCO₃ solution (2×) and dried with Na₂SO₄ (anhydrous). The solvent was evaporated in vacuo to produce a white foam containing products of reaction.

Method 2: Compound E-1 (265 mg, 0.33 mmol) was taken up in 10 mL dichloromethane and stirred. Separately 1.6 eq of methoxyacetic acid and 1.6 eq of 1,1′-Carbonyldiimidazole were solved in dichloromethane (5 mL) and stirred over 20 min. Both solutions were unified and stirred continually at room temperature. The reaction solution was washed with 5% citric acid three times to extract the product. Acidic solution was then washed with ethyl acetate (2×) and afterwards neutralized with Na₂CO₃. Product was extracted with ethyl acetate (3×). The solution was washed with a saturated solution of sodium chloride (2×), water (2×) and dried over anhydrous Na₂SO₄. The solvent was evaporated in vacuo to produce a white foam containing E-12 (238 mg, 90%).

Workup 1: Column chromatography over silica gel was carried out to separate different products. As eluent a mixture of chloroform, 2-propanol and ammonia in methanol (60:1:1) was used. The solvent was evaporated in vacuo.

Workup 2: Column chromatography over silica gel was carried out to separate different products. As eluent a mixture of cyclohexane, acetone (3:1) with 0.5% triethylamine was used. The solvent was evaporated in vacuo.

TABLE 21
Typical Products from the Acylation Procedures (2)

| Cpd | Synthesis | | | Reaction Condition | Degree of | | |
|---|---|---|---|---|---|---|---|
| Entry | Method | Carboxylic acid | ALC | (i.e. Workup) | Substitution | Yield | MS |
| E-545 | 1 | Cyclopropanecarboxylic acid | A-1 | Workup 1 | 1x | 30% | 817 |
| E-546 | 1 | Cyclobutanecarboxylic acid | A-1 | Workup 1 | 1x | 10% | 830 |
| E-546, | 1 | Cyclobutanecarboxylic acid | A-1 | Workup 1 | 1x, 2x, 3x | 22.5% | 830, |
| E-560, | | | | | | | 913, |
| E-561 | | | | | | | 995 |
| E-547 | 1 | Nicotinic acid | A-1 | Workup 2 | 1x | 10.3% | 854 |
| E-547, | 1 | Nicotinic acid | A-1 | Workup 2 | 1x, 2x | 11.8% | 854, |
| E-562 | | | | | | | 959 |
| E-551 | 1 | Methoxyacetic acid | A-1 | Workup 1 | 1x | 4.4 | 820 |
| E-14, | 1 | Methoxyacetic acid | A-1 | | 2x, 3x, 4x | Reaction | 893, |
| E-22, | | | | | | solution | 965, |
| E-559 | | | | | | | 1037 |
| E-12 | 2 | Methoxyacetic acid | E-1 | none | 1x | 90% | 866 |
| E-81 | 1 | 3-Phenylpropionic acid | A-1 | Workup 1 | 1x | 30% | 881 |
| E-544 | 1* | lndole-3-propionic acid | A-1 | Workup 1 | 1x | 45% | 920 |

*instead of 1,1′-carbonyl diimidazole, HATU was used as coupling agent.

### Example 52. Synthesis of E-541 and E-542

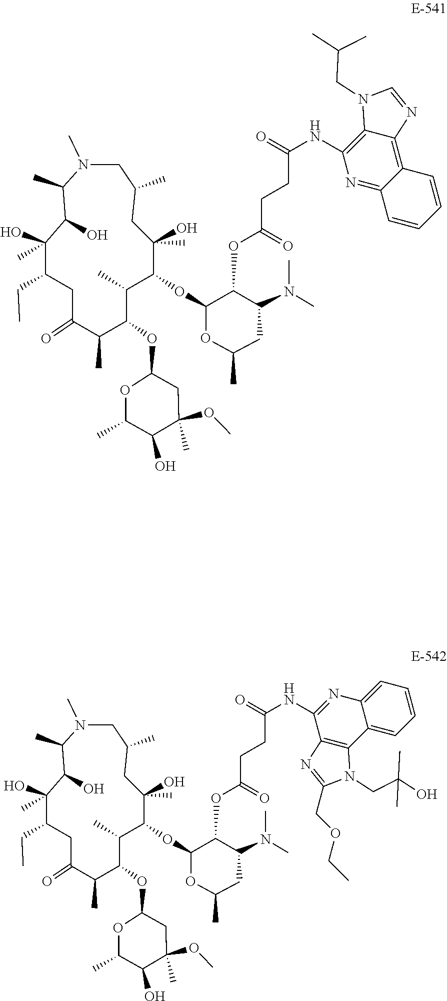

E-27 (1.2 mmol) and Quinoline-amine (1 eq) was taken up in DCM (5 mL). To this was added HATU (1.2 eq) neat. The reaction was stirred overnight at room temperature. Reaction was very sluggish an additional 0.5 eq of HATU was added. Reaction was stirred for 2 days or disappearance of starting material was observed (TLC or MS). Reaction solution was removed in vacuo and the crude material directly purified by chromatography to get the desired product.

TABLE 22
Decoration of ALC (E-27) with potential TLR-agents

| Cpd | | Degree of | | |
|---|---|---|---|---|
| Entry | Quinoline | Substitution | Yield | MS |
| E-541 | Imiquimod | 1 | 35% | 1071 |
| E-542 | Resiquimod | 1 | 25% | 1145 |

### Example 53. Examples of Polyamines as ALC

These ALCs can be prepared by reacting symmetrical or unsymmetrical di- or poly-epoxides with secondary amines. This will provide ALCs that contains common structural element of 2 or more alcohols, vicinally neighbored by a tertiary amine. Some polyamines are also commercially available.

Alternatively, ALCs can be prepared by reacting epoxides with diethanolamine. The reaction products are containing 2-hydroxy tertiary amines.

### General Procedure for Preparation of Some Polyamine ALC Bearing Hydroxy Functionalities:

Polyamine (1 mmol) containing at least 2 NH-functions and the epoxide are mixed and heated without solvent to 80° C. Excess epoxide can be removed by column chromatography selectively. Products are sufficient, when at least 2 tertiary ß-hydroxyamines are present.

TABLE 23
Examples of Polyamine ALCs

| | | | Eq. of | Reaction | [M + H]⁺ | | |
|---|---|---|---|---|---|---|---|
| Entry | Amine | Epoxide | Epoxide | time | Product | Yield | Remark |
| 1 | hexamethylene diamine | 1,2- | 4.2 | 40 h | 966 | n.d. | product contains a |
| | | epoxytetradecane | | | | | small amount of triple |
| | | | | | | | alkylation |
| 2 | bis- | cyclohexene | 12 | 240 h | 608 | n.d. | main product is |
| | (hexamethylene)triamine | oxide | | | | | tetraalkylated |

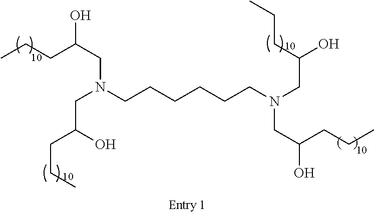

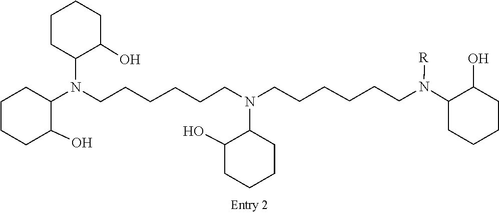

R = H(tetraalkylated)
= 2-hydroxycyclohexal (pentaalkylated)

Reactions of Polyepoxides with Amines:

Corresponding polyepoxide (1 mmol) is mixed with 1.05 mmol secondary amine per epoxide function and heated to 80° C. without solvent for 12 h.

TABLE 24
Further Examples Polyamine ALCs

| | | | [M + H]⁺ | |
|---|---|---|---|---|
| Entry | Epoxide | Amine | Product | Yield |
| 1 | 1,2,7,8-diepoxyoctane | morpholine | 317 | 100% |
| 2 | Diglycidyl ethylenglycol | morpholine | 377 | 90% |

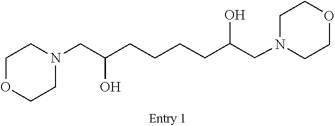 [SMILES: OC(CCCCC(O)CN1CCOCC1)CN1CCOCC1]

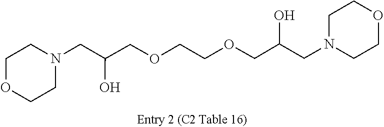 [SMILES: OC(COCCOCC(O)CN1CCOCC1)CN1CCOCC1]

### Synthesis of E-552

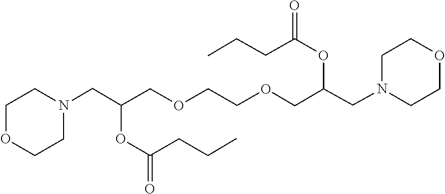 [SMILES: CCCC(=O)OC(COCCOCC(CN1CCOCC1)OC(=O)CCC)CN1CCOCC1]

1.45 g of 1,10-dimorpholino-2,9-dihydoxy-4,7-dioxadecane are combined with 1.5 ml of butyric anhydride in 5 ml of chloroform. After stirring for 1 h, MS indicates complete conversion ([M+H]*=517). The mixture is extracted with 2 N KOH, saturated aqueous sodium bicarbonate solution and brine, dried and chromatographed over silica gel to obtain 1.57 g of the target compound (72%).

### Example 54. Substitution of Polyamine ALC (See Table 1, Entry A-19)

### Substitution ALC A-19.1/A-20.1/A-20.2

Method 1: N-Hydroxyalkyl compound (5 mmol) was suspended in excess carboxyl acid anhydride (>100 mmol, >20 eq.) in a round bottom flask while stirring (magnetic stir bar, 500 rpm). The mixture was cooled in an ice bath and sulfuric acid (>96%, 3 drops) was carefully added as catalyst. Stirring was continued until a clear solution was obtained. When ESI-MS indicated full conversion of starting materials the reaction mixture was poured on ice. The system was stirred for 2 or more hours in order to hydrolyze any anhydride. The mixture was neutralized by addition of sodium bicarbonate and extracted with dichloromethane (3×). Separation of organic phase, drying over sodium sulfate and evaporation of any volatiles in vacuo yielded the product as colorless oil.

Method 2. Carboxylic acid (1.5 eq per hydroxyl group) was placed into a round bottom flask along with a stir bar and carbonyl diimidazole (CDI, 1.6 eq per hydroxyl group). Dichloromethane (DCM, 10 mL per gram carboxylic acid) was added carefully while stirring (500 rpm) at ambient temperature. Immediate formation of carbon dioxide indicated conversion of corresponding acid to the acyl donor (caution: too quick CO₂ formation may result in strong foaming. Do not seal the flask!). After a couple of minutes, a clear solution was obtained and stirring was continued for 15 minutes. N-Hydroxyalkyl compound (5 mmol) was added at ambient temperature and the reaction mixture was stirred overnight. When ESI-MS indicated full conversion of starting materials the reaction was quenched by addition of methanol, converting excess acyl donor to methyl ester. The system was diluted by addition of further DCM and was subject to extraction with saturated sodium bicarbonate solution (3×). Separation of organic phase, drying over sodium sulfate and evaporation of any volatiles in vacuo yielded the product as colorless oil.

TABLE 25
Substitution of ALC A-19.1/A-20.1/A-20.2

| Compound | Synthesis | | Degree of | | MS |
|---|---|---|---|---|---|
| Entry | Method | ALC | Substitution | Yield | [M + H⁺ |
| E-524 | 1 | A-19.1 | Di acyl | 85% | 259, M + H⁺ |
| E-576 | 1 | A-19.1 | Mono acyl | 23% | 217, M + H⁺ |
| E-525 | 1 | A-19.1 | Di acyl | 94% | 287, M + H⁺ |
| E-577 | 1 | A-19.1 | Mono acyl | 12% | 231, M + H⁺ |
| E-526 | 2 | A-19.1 | Di acyl | 87% | 315, M + H⁺ |
| E-578 | 2 | A-19.1 | Mono acyl | 45% | 245, M + H⁺ |
| E-527 | 1 | A-19.1 | Di acyl | 56% | 315, M + H⁺ |
| E-529 | 1 | A-19.1 | Di acyl | 8% | 343, M + H⁺ |
| E-530 | 2 | A-19.1 | Di acyl | 76% | 499, M + H⁺ |
| E-538 | 1 | A-20.1 | Tri acyl | 85% | 299, M + Na⁺ |
| E-537 | 1 | A-20.1 | Tri acyl | 76% | 383, M + Na⁺ |
| E-580 | 1 | A-20.2 | Tri acyl | 61% | 391, M + Na⁺; |
| | | | | | 368, M⁻ |

### Substitution ALC A-19.2

1,1′-Carbonyldiimidazole was dissolved in dichoromethane (dry, 25 mL) and to this was added the carboxylic acid slowly at room temperature. The solution was stirred at room temperature before a suspension of N;N,N′,N′-tetrakis (2-hydroxyethyl)-ethylendiamine (A-19.2) in dichloromethane (dry, 5 mL) was added in one portion at room temperature and the mixture was stirred at RT. The reaction mixture was filled into a separation funnel and washed. The organic phase was dried (Na₂SO₄) and concentrated to dryness in vacuo.

The crude product was purified by column chromatography.

Eluent:CHCl₃:Isopropanol:NH3 (7 M in MeOH)=60:1:1.

TABLE 26
Substitution of ALC A-19.12

| | | | | | Time for | | | |
|---|---|---|---|---|---|---|---|---|
| | | | Amount | | stirring | | | |
| Cpd | | Amount | Carboxylic acid | Amount | carboxylic acid | | Washing | |
| Entry | ALC | (A-19.2) | (mg) | CDI | and CDI | Reaction Time | steps | Yield |
| E-531 | A-19.2 | 521 mg | Glacial acetic | 2.49 g | 35 min | 22 h 30 min | NaHCO₃ | 297 mg |
| | | (73.4%) | acid | 15.35 mmol | | | (2 × 20 mL) | (45%) |
| | | 1.64 mmol | 800 μL | | | | | |
| | | | 13.99 mmol | | | | | |
| E-532 | A-19.2 | 498 mg | Propionic acid | 2.46 g | 20 min | 1.5 h with | Water | 144 mg |
| | | (73.4%) | 943 μL | 15.17 mmol | with argon | argon stream | (1 × 20 mL) | (20%) |
| | | 1.55 mmol | 12.6 mmol | | stream | 69 h 45 min | sat. | |
| | | | | | | under argon | NaHCO₃ | |
| | | | | | | atmosphere | (2 × 20 mL) | |
| E-533 | A-19.2 | 495 mg | Butyric acid | 2.51 g | 35 min | 46 h 45 min | NaHCO₃ | 20 mg |
| | | (73.4%) | 1.2 mL | 15.45 mmol | | | (2 × 20 mL) | (pure) |
| | | 1.54 mmol | 13.55 mmol | | | | | 224 mg |
| | | | | | | | | (with |
| | | | | | | | | impuri- |
| | | | | | | | | Ties |
| | | | | | | | | Overall |
| | | | | | | | | yield: (19%) |

### Analytical Data:

### E-531:

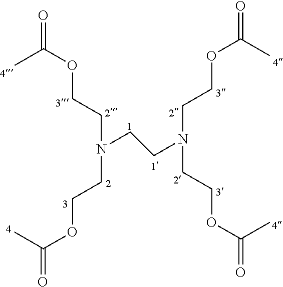 [SMILES: CC(=O)OCCN(CCOC(C)=O)CCN(CCOC(C)=O)CCOC(C)=O]

ESI-MS (positive): m/z=405.2 [M+H]⁺, 427.1 [M+Na]⁺

Purity according to HPLC (ELSD): >99.9%

¹H-NMR (300 MHz, CDCl₃): 1.98 (s, 12H, 4-H, 4′-H, 4″-H, 4′″-H), 2.57 (s, 4H, 1-H, 1¹H), 2.72 (t, J_{2,3} and J_{2′,3′}, J_{2″,3″}, J_{2′″,3′″}=6.04, 8 H, 2-H, 2′-H, 2″-H, 2′″-H), 2.72 (t, J_{3,2} and J_{3′,2′}, J_{3″,2″}, J_{3′″,2′″}=4.04, 8 H, 2-H, 2′-H, 2″-H, 2′″-H).

¹³C-NMR (75 MHz, CDCl₃): 20.77 (q, C-4, C-4′, C-4″, C-4′″), 53.15 (t, C-2, C-2′, C-2″, C-2′″), 53.44 (t, C-1, C-1′), 62.43 (t, C-3, C-3′, C-3″, C-3′″), 170.74 (s, 4×C═O).

### E-532:

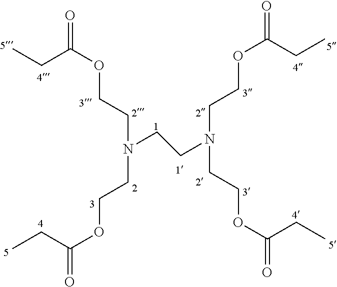 [SMILES: CCC(=O)OCCN(CCOC(=O)CC)CCN(CCOC(=O)CC)CCOC(=O)CC]

ESI-MS (positive): m/z=461.2 [M+H]+, 483.3 [M+Na]+

Purity according to HPLC (ELSD): >99.9%

¹H-NMR (300 MHz, CDCl₃): 1.08 (t, J_{5,4}, J_{5′,4′}, J_{5″,4″}, J_{5′″,4′″}=7.6 Hz, 12H, 5-H, 5′-H, 5″-H, 5′″-H), 2.27 (q, J_{4,5}, J_{4′,5′}, J_{4″,5″}, J_{4′″,5′″}=7.6 Hz), 2.57, 4-H, 4′-H, 4″-H, 4′″2.60 (s, 4H, 1-H, 1′H), 2.74 (t, J_{2,3} and J_{2′,3′}, J_{2″,3″}, J_{2′″,3′″}=6.04, 8 H, 2-H, 2′-H, 2″-H, 2′″-H), 4.07 (t, J_{3,2} and J_{3′,2′}, J_{3″,2″}, J_{3′″,2′″}=6.04, 8 H, 2-H, 2′H, 2″-H, 2′″-H).

¹³C-NMR (75 MHz, CDCl₃): 8.96 (q, C-5, C-5′, C-5″, C-5′″), 27.44 (t, C-4, C-4′, C-4″, C-4′″), 53.22 (t, C-2, C-2′, C-2″, C-2′″), 53.54 (t, C-1, C-1′), 62.36 (t, C-3, C-3′, C-3″, C-3′″), 172.20 (s, 4× C═O).

### E-533:

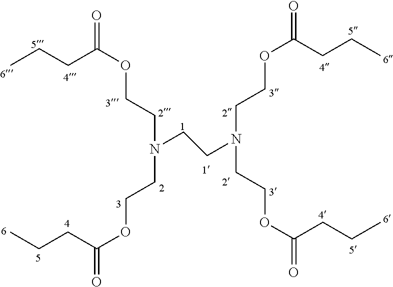 [SMILES: CCCC(=O)OCCN(CCOC(=O)CCC)CCN(CCOC(=O)CCC)CCOC(=O)CCC]

ESI-MS (positive): m/z=617.3 [M+H]⁺, 539.3 [M+Na]⁺

Purity according to HPLC (ELSD): >99.9%

¹³C-NMR (75 MHz, CDCl₃): 13.53 (q, C-6, C-6′, C-6″, C-6′″) 18.26 (t, C-5, C-5′, C-5″, C-5′″), 36.00 (t, C-4, C-4′, C-4″, C-4′″), 53.21 (t, C-2, C-2′, C-2″, C-2′″), 53.45 (t, C-1, C-1′), 62.24 (t, C-3, C-3′, C-3″, C-3′″), 173.36 (s, 4× C═O):

### Substitution ALC A-19.3

H-L-orn(Boc)2CT Resin (0.68 mmol/g, 100-200 mesh, 2.99 g, 2.07 mmol) was filled into a 20 mL syringe with frit. Dichloromethane (dry, 10 mL), MeOH (2 mL) and diisopropylethylamine (2 mL) are added to the resin for endcapping. The mixture was shaken at room temperature for 30 min, then the liquid was sucked off and the resin was washed (3× dimethylformamide 15 mL, 1× diethylether 15 mL).

The resin was filled into a 100 mL round bottom flask. DMF (25 mL) was added and the resin was swollen for 5 min. Then diisopropylethylamine (3.8 mL, 22.3 mmol) and 2-bromoethanol (1.434 mL, 20.3 mmol) were added subsequently at room temperature. The reaction mixture was stirred at 60° C. (bath temperature) for 24 h.

The resin was filled into a 20 mL syringe with frit and was washed: 4× dimethylformamide (20 mL), 3× methanol (20 mL), 3× dichloromethane (20 mL), 3× diethyl ether (20 mL).

Half of the resin (1.035 mmol) was filled into a 20 mL syringe with frit.

Valeric acid (568 μL, 5.69 mmol) was added to a mixture of dimethylformamide/dichloro-methane 1:1 (10 mL). HOBt*H₂0 (870 mg, 5.69 mmol) was added and mixture was stirred at room temperature for 5 min before diisipropylcarbodiimide (881 μL, 5.69 mmol) was added. Stirring at room temperature was continued for 10 min, then the whole mixture was added to the resin and the resin was shaken at room temperature for 5 h.

The liquid was sucked off and the resin was washed:

4× dimethylformamide (10 mL), 3× methanol (10 mL), 3× dichloromethane (10 mL), 3×diethyl ether (10 mL).

A test cleavage showed the product by mass spectrometry

ESI-MS (positive): m/z=261.1 [M+H]⁺

### Example 55. Synthesis of 2′-O-(2-Ferrocenyl) acetyl-azithromycin E-549

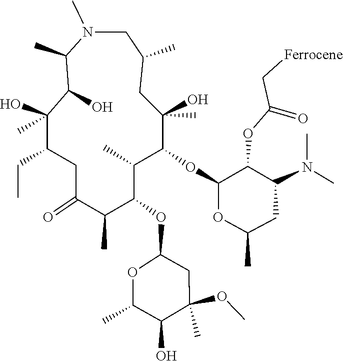

ALC A-1 (0.25 mmol) was dissolved in dry dichloromethane (5 mL). To this was added EDCI (2 eq., 0.5 mmol) and 2-ferrocenyl acetic acid (1.1 eq., 0.28 mmol). The reaction was stirred overnight at room temperature. The solvent was removed in vacuo and the resulting white amorphous foam. The resulting crude product was purified by column chromatography with a gradient starting at 10% of acetone in cyclohexane (0.2% Et₃N).

### Example 56. Synthesis of E-258

Compound A-11/E-16 was dissolved in dry dichloromethane (DCM) in a round bottom flask equipped with magnetic stir bar. Penta-O-acetyl-α-D-mannopyranoside (1.2 eq) was added and the system was cooled in an ice bath while stirring (300 rpm). Catalytic amount of boron trifluoride diethyl ether complex was carefully added dropwise and the system was allowed to warm up while stirring overnight. Upon dilution with further DCM the mixture was subject to extraction with saturated sodium bicarbonate solution (3×). Separation of organic phase, drying over sodium sulfate and evaporation of any volatiles in vacuo yielded the product as colorless oil or beige to off-white foam. [M+H]⁺ m/z 907 The reaction conditions also produced the des-cladinosyl product E-600.

### Example 57. Synthesis of E-550

350 mg of compound E 77 are dissolved with 15 ml of carbon disulfide. 250 mg of sulfur are added and the mixture is stirred for 7 days. The mixture is extracted with 5% aqueous citric acid solution. The aqueous extract is combined with 10 ml of ethyl acetate and made alkaline by addition of sodium carbonate with intense stirring. The organic phase is separated, washed with brine, dried over sodium sulfate and concentrated in vacuo to yield 280 mg of a product, that contains various higher sulfides along with some starting material, as indicated by mass spectrometry ([M+H]⁺=969, 1001, 1033, 1065).

### Example 58. Pharmacokinetics

The distribution of compounds to target organs is of specific importance to the efficacy of anti-infective compounds. To determine distribution the compounds are formulated and administered to a suitable animal model. Compounds were administered p.o. 10 mg/kg in 2% citric acid in BALBc and organs were recovered at 6 h. Organs were extracted in Acetonitrile (6× volume of the sample), centrifuged at 14000 g for 5 minutes. Samples were analysed by LCMSMS (SCIEX 4500). Data are the mean of 3 animals.

### Example 59. Selection of ALC Via Concentration into Immune Cells

The distribution of compounds to target cells is of specific importance to the efficacy of anti-infective compounds. To determine uptake the compounds are dissolved in DMSO or citric acid and mixed with whole blood, plasma or cell medium. To these solutions are added cultured macrophages, cultured immune cells, bone marrow derived macrophages, peritoneal macrophages or buffy coat cells. The mixture is incubated at 37° C. for 1, 2, or 3 hours. After incubation, the immune cells are separated from the medium and the concentration of the compounds is determined by extraction in Acetonitrile (6× volume of the sample), followed by centrifugation at 14000 g for 5 minutes. The resulting extracts are analyzed by LCMSMS (SCIEX 4500 in positive mode). Data are the mean of 3 animals.

### Example 60. Synthesis of CSY1019

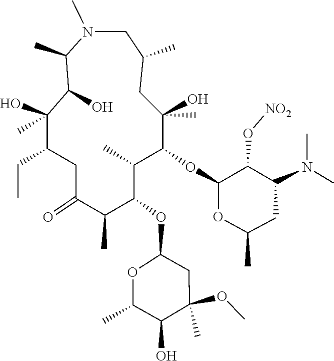

All glassware were initially oven-dried and cooled under an argon atmosphere.

Dry acetonitrile (10 mL) was added into a round bottom flask under an argon atmosphere. The reaction was cooled to −10° C. in a NaCl-ice-bath. AgNO₃ (284 mg; 1.67 mmol) and Pivaloyl chloride (174 μL; 1.42 mmol) were added respectively. After 30 minutes, azithromycin was added and the reaction was allowed to stir at 0° C. for about 3 hours (Reaction progress was monitored via MS). The reaction solution was filtered through Celite and the residue was washed with additional acetonitrile. The ACN solution were poured into an ice-water mixture with one spoon of NaHCO₃ under constant stirring (pH 8). The mixture was allowed to warm to room temperature until the ice has melted. The aqueous solution was extracted with ethyl acetate (3×). Then the combined organic layers were washed with saturated aqueous NaCl solution, dried with anhydrous Na₂SO₄ and evaporated in vacuo to get a light-yellow foam. Crystallization with MeOH/Water provided white crystals (657 mg; 61% yield).

### Example 61. Formulation with an ALC Compound

The action of a compound can be improved by the addition of an unconjugated ALC compound, for example azithromycin. Compounds such as E4 or E5 may have useful oral doses in human subjects in the range of 0.1 to 10 mg. These may be conveniently included in mixtures of azithromycin at final doses between 250 to 500 mg. In one formulation, 1 mg of E4 or E5 is combined with 250 or 500 mg of azithromycin in pill or capsule form. Similarly, compounds E87, 88 and 89 can be mixed with either azithromycin or hydroxychloroquine. 1 to 100 mg of compounds E87, 88 and 89 are mixed with 250 or 500 mg of azithromycin in pill or capsule form. Alternatively, 1 to 100 mg of compounds E87, 88 and 89 are mixed with 200 or 300 mg of hydroxychloroquine in pill or capsule form. Similarly, compounds E87, 88, 89 or 300 can be mixed with either azithromycin or Camostat. 1 to 100 mg of compounds E87, 88, 89 or 300 are mixed with 250 or 500 mg of azithromycin in pill or capsule form. Alternatively, 1 to 100 mg of compounds E87, 88 and 89 are mixed with 200 or 300 mg of camostat in pill or capsule form.

### Example 62. Formulation with an ALC Compound with Zinc

Zinc orotate is a form of zinc that is easily absorbed by the oral route. Zinc orotate is formulated with ALC compounds to improve anti-viral effects. 300 mg of hydroxychloroquine is mixed with 1 to 10 mg of compounds E87, 88 or 89. To this mixture is added 5 to 60 mg of zinc orotate. Alternatively, 1 to 10 mg of compounds E87, 88, 89 or 300 is added to 5 to 60 mg of zinc orotate.

To this mixture can be added or given simultaneously camostat mesylate 200 to 300 mg or nafamostat mesylate 30 to 50 mg.

Alternatively, 1 mg of E4 or E5 is combined with 250 or 500 mg of azithromycin and/or 5 to 60 mg of zinc orotate in pill or capsule form. To this mixture can be added or given simultaneously camostat mesylate 200 to 300 mg or nafamostat mesylate 30 to 50 mg.

In both cases, the ALC compound improves the action of camostat or nafamostat by reducing the efficiency of endosomal cathepsin reactions which are not inhibited by camostat or nafamostat, while also inhibiting the action of viral proteases and increasing viral killing through the induction of iNOS.

### Example 63. Formulation with an ALC Compound with Camostat and Zinc

Zinc orotate is a form of zinc that is easily absorbed by the oral route. Zinc orotate is formulated with ALC compounds (e.g., E5 or E300) to improve anti-viral effects. 300 mg of hydroxychloroquine is mixed with 5 to 60 mg of zinc orotate and Camostat mesylate 200 to 300 mg. Alternatively, 250 mg of azithromycin and 5 to 60 mg of zinc orotate is mixed with camostat mesylate 300 mg in pill or capsule form.

In both cases, the ALC compound improves the action of camostat or nafamostat by reducing the efficiency of endosomal cathepsin reactions which are not inhibited by camostat.

Zinc orotate is formulated with ALC compounds (e.g., E5 or E300) to improve anti-viral effects.

### Example 64. Formulation with an ALC Compound

The action of a compound can be improved by the addition of an unconjugated ALC compound, for example azithromycin. 5 mg of E542 is combined with 250 or 500 mg of azithromycin in pill or capsule form. Similarly, 5 mg of E542 is combined with 200 or 300 mg of hydroxychloroquine in pill or capsule form.

### Example 65. Testing of mixed therapy

The mixture of example 63 may be tested for efficacy in a model of pneumonia in mice. Infections of the human pneumovirus respiratory syncytial virus (RSV) can be modeled using the mouse pneumonia virus of mice (PVM). On day 0, animals are infected with 2×10⁴ copies of PVM diluted in 20 μL RPMI-1640 intra-nasally under 2% isoflurane anesthesia. The animals are treated p.o. with the equivalent of 10 mg/kg azithromycin, 10 mg/kg camostat, 2 mg/kg Zinc orotate and 10 mg/kg hydoxychloroquin in a mixture for 3 days vs. Vehicle or the substances alone. At termination, the left lung was removed and flash-frozen in liquid nitrogen for homogenization for quantitation of expression.

Lavage fluid (NaCl 0.9%/EDTA 0.6 mmol/L 0.5 mL) was obtained from the right lung lobe for viral plaque count and for estimation of cells and cytokines. After the lavage, the lung was removed and fixed in 10% formalin for histological studies. Mice subject to the treatment with the mixture have higher survival and body weight, but fewer infiltrating cells and cytokines vs. the animals receiving substance alone or vehicle.

Infections of Staphylococcus aureus can serve as models of human pneumonia or ARDS. On day 0, animals are infected with 1×10⁷ S. aureus CFU in 20 μL saline solution intra-nasally under 2% isoflurane anesthesia. The animals are treated p.o. with the equivalent of 10 mg/kg azithromycin, 10 mg/kg camostat, 2 mg/kg Zinc orotate and 10 mg/kg hydoxychloroquin in a mixture once two hours after infection vs. Vehicle or the substances alone. At 24 h the animals are euthanized and the left lung recovered for quantification of remaining bacteria. Lavage fluid (NaCl 0.9%/EDTA 0.6 mmol/L 0.5 mL) was obtained from the right lung lobe for estimation of cells, notably neutrophils and cytokines. After the lavage, the lung was removed and fixed in 10% formalin for histological studies. Mice subject to the treatment with the mixture have higher survival and body weight, but fewer infiltrating cells and cytokines vs. the animals receiving substance alone or vehicle.

### Example 66. Testing of Mixed Therapy in a Clinical Trial

The mixture of example 63 may be tested for efficacy in a clinical trial in mild to severe Covid-19 patients.

Initial indications of efficacy can be obtained from an open label observational trial of a mixture of 1 mg E4, camostat 300 mg, zinc orotate 60 mg, 300 mg hydroxychloroquine and 250 mg azithromycin vs. standard of care (e.g. 300 mg hydroxychloroquine and 250 mg azithromycin). Patients diagnosed with SARS-CoV-2 pneumonia according to WHO interim guidance, and who were classified as mild to severe are included in the trial. Treatment is for up to 14 days. Endpoints include viral counts by nasal swab, admission to the intensive care unit (ICU) and the proportions of patients with detectably viral genomes. Alternatively, endpoints include viral counts by nasal swab, admission to the intensive care unit (ICU) and antibody production.

### Example 67. Zinc Complexes of ALC Compounds

ALC compounds can be conveniently prepared as zinc complexes.

940 mg of Zinc acetate dihydrate (Zn(OAc)₂*2H₂O) were suspended in 30 ml of a 3+1 mixture of THF and methanol. 7.2 g of azithromycin were added, and the solution was allowed to concentrate by evaporation.

880 mg of Zinc acetate dihydrate (Zn(OAc)₂*2H₂O) were suspended in 40 ml of THF. 3.0 g of azithromycin were added, the mixture was concentrated by distillation to approx. 10 ml and then allowed to cool and concentrate further at RT by evaporation. 560 mg of ZnCl₂ are dissolved with 20 ml of THF. 1.49 g of hydroxychloroquin are dissolved with 20 ml of THF and added slowly to the ZnCl₂-solution. The precipitate is stirred for 24 h and isolated by filtration.

### Example 68. Interaction of ALC Compounds and Protease Inhibitors

ALC compounds can be used alone or in combination with other compounds. These other compounds may include protease inhibitors or which one example is camostat. Camostat inhibits the TMPRSS2 protease that can activate the spike protein for cell entry. In addition, compounds that stimulate anti-viral defense such as Zn ions, glutathione, citrulline and arginine may be considered as interaction partners.

Interactions may be tested by providing the substances to cells that have been infected with a virus strain of appropriate virulence. For example, CaCo2 cells (human colon carcinoma) may be infected with SARS-CoV-2 expressing marker proteins such as GFP or luciferase and the amount of virus production quantified in terms of fluorescence or luminescence respectively. 10,000 cells in 100 μL are seeded to a microtitre plate well. One day later, compounds are added in 50 μL medium and cells are infected with 50 μL virus suspension titrated for a final concentration of 1 virus particle per cell. After 2 days, virus production is quantified in that cells are treated with 4% PFA in saline containing Hoechst nuclear stain. The level of Hoechst staining is an estimate of cell viability and number. Fluorescence or luminescence is an estimate of virus production. A typical reading for fluorescence in such an assay is normalized to Hoechst staining of the nuclei to provide an estimate of virions/cell. For untreated cells, the ratio is in the range of 0.2 which indicates that viral fluorescence is ⅕ of nuclear fluorescence.

Comparing compounds in this way results in effects as follows:

TABLE 1
Example 68, dose response to inhibitors alone or with addition of
other substances as indicated. Fluorescence ratio (untreated = 0.2)

| Conc. | | E5 + GSH + Citrulline | | Camostat + | Camostat + | |
|---|---|---|---|---|---|---|
| (μM) | E5 | (2 mM)* | Camostat | E5 (0.6 μM)* | E298 (5 μM)* | E298 |
| 10 | 0.01 | 0.00 | 0.02 | 0.00 | 0.01 | 0.13 |
| 5 | 0.00 | 0.00 | 0.07 | 0.00 | 0.01 | 0.24 |
| 2.5 | 0.01 | 0.02 | 0.03 | 0.01 | 0.02 | 0.18 |
| 1.25 | 0.09 | 0.04 | 0.01 | 0.02 | 0.03 | 0.19 |
| 0.625 | 0.14 | 0.20 | 0.08 | 0.02 | 0.02 | 0.17 |
| 0.312 | 0.22 | 0.23 | 0.09 | 0.03 | 0.04 | 0.17 |

*Dose response to first named compound with addition of the second or third compound at the fixed concentration (indicated).

TABLE 2
Example 68, Production of viral protein according to the
method in this example, in response to inhibitors in medium
at a final concentration of 10 μM. Low values are indicative
of inhibition of viral protein production.

| | SARS-CoV-2 viral protein production in CaCo2 cells - |
|---|---|
| Compound | effect of each substance at 10 μM (% of untreated) |
| A-23 | 44 |
| A-24 | 35 |
| A-25 | 34 |
| Camostat | 2 |
| E5 | 18 |
| E11 | 32 |
| E-18 | 49 |
| E-19 | 34 |
| E-39 | 20 |
| E47 | 1 |
| E50 | 45 |
| E-86-i | 49 |
| E-86-d | 60 |
| E-86-e | 46 |
| E-86-f | 51 |
| E-86-g | 18 |
| E300 | 1 |

### Example 69. Testing of Mixed Therapy in a Clinical Trial

The mixture of example 68 may be tested for efficacy in a clinical trial in mild to severe Covid-19 patients.

Initial indications of efficacy can be obtained from an open label observational trial of a mixture of 1 mg E5 and camostat 300 mg orally, vs. E5 1 mg alone or standard of care. Patients newly diagnosed with SARS-CoV-2 that are PCR positive and symptomatic are included in the trial. They are allocated to groups, blood samples taken for viremia and cytokines, and treated with oral formulations of the above substances. Endpoints include duration of signs, viral counts by nasal swab and blood quantitative PCR, admission to the intensive care unit (ICU) and survival.

### Example 70. Testing of Mixed Therapy

The efficacy of E5 or mixture of E5, E300 and Camostat may be tested for efficacy in a model of pneumonia in mice.

Infections of the human pneumovirus, respiratory syncytial virus (RSV) can be modeled using the mouse pneumonia virus of mice (PVM). On day 0, animals are infected with 2×10⁴ copies of PVM diluted in 20 μL RPMI-1640 intra-nasally under 2% isoflurane anesthesia. The animals are treated p.o. with the equivalent of 0.01, 0.1 or 1 mg/kg E5 alone or with 10 mg/kg camostat or 2 mg/kg Zinc for 3 days vs. Vehicle or the substances alone. At termination, the left lung is removed and flash-frozen in liquid nitrogen for homogenization for quantitation of expression.

Lavage fluid (NaCl 0.9%/EDTA 0.6 mmol/L 0.5 mL) is obtained from the right lung lobe for viral plaque count and for estimation of cells and cytokines. After the lavage, the lung was removed and fixed in 10% formalin for histological studies. Mice subject to the treatment with the mixture have higher survival and body weight, but fewer infiltrating cells and cytokines vs. the animals receiving substance alone or vehicle.

Infections of Staphylococcus aureus can serve as models of human pneumonia or ARDS. On day 0, animals are infected with 1×10⁷ S. aureus CFU in 20 μL saline solution intra-nasally under 2% isoflurane anesthesia. The animals are treated p.o. with the equivalent of 0.01, 0.1 or 1 mg/kg E5 alone or with 10 mg/kg camostat or 2 mg/kg Zinc in a mixture once two hours after infection vs. Vehicle or the substances alone. At 24 h the animals are euthanized and the left lung recovered for quantification of remaining bacteria. Lavage fluid (NaCl 0.9%/EDTA 0.6 mmol/L 0.5 mL) was obtained from the right lung lobe for estimation of cells, notably neutrophils and cytokines. After the lavage, the lung was removed and fixed in 10% formalin for histological studies. Mice subject to the treatment with the mixture have higher survival and body weight, but fewer infiltrating cells and cytokines vs. the animals receiving substance alone or vehicle.

### Example 71

### Synthesis of E-328 (Tripropionate of Compound 14.2)

1.13 g of 14.2 were dissolved with 12 ml of dichloromethane. 400 μl of propionic anhydride were added and the mixture was stirred for 4 days at room temperature. 200 μl more of propionic anhydride were added and stirring was continued for 7 days. The reaction mixture was extracted (2×) with 5% aq. citric acid solution (20 ml each). The aqueous phases were combined, mixed with 30 ml of ethyl acetate and neutralized by portion-wise addition of solid sodium carbonate. When gas evolution ceases, the phases were separated. The organic phase was washed with water and brine, dried over sodium sulfate, concentrated and purified by flash chromatography over silica gel (cyclohexane—acetone, both containing 0.25% of triethylamine). The yield is 803 mg of the target compound E-328 ((m+H)/z=977, 1st fragment=819=loss of cladinose).

### Example 72

Nitration of E-328

Method 1 of Example 1 was applied to E-328 instead of azithromycin. initial weight: 256 mg

yield: 59 mg of compound E-330-a ((m+H)/z=1022).

### Example 73: Propionylation of A-14.2

Chemicals:

| | | | |
|---|---|---|---|
| A-14.2 | mw = 790.5 g/mol | m = 0.6 g | n = 0.75 mmol |
| Propionic | mw = 130.1 g/mol | V = 72.5 μL | n = 0.90 mmol |
| anhydride | | | |
| Pyridine | mw = 79.1 g/mol | V = 105.5 μL | n = 0.83 mmol |
| Dichloromethane | V = 80 ml | | |

TLC: Cyclohexane, Acetone (3:1) with 0.25% Triethylamine

Procedure:

A-14.2 (0.6 g, 0.75 mmol) was dissolved in dichloromethane. Afterwards the solution was cooled with ice and 1.2 eq of pyridine was added, followed by 1.1 eq of propionic anhydride. Solution was stirred at room temperature overnight. Reaction was monitored via TLC and MS. Work up was done after three days, when sufficient product was detected. To extract product liquid-liquid extraction was performed. DCM solution was worked up with a saturated solution of ammonium chloride three times, followed by water three times. Organic phase was then dried with sodium sulphate and solvent evaporated to carry out white solid powder.

compound E-383 Yield 72% 95.7% purity

### Example 74: Synthesis of propionylated A-14.1

Chemicals:

| | | | |
|---|---|---|---|
| A-14.1 | mw = 790.5 g/mol | m = 1.2 g | n = 1.5 mmol |
| Propionic anhydride | mw = 130.1 g/mol | V = 145 μL | n = 1.80 mmol |
| Pyridine | mw = 79.1 g/mol | V = 211 μL | n = 1.65 mmol |
| Dichloromethane | V = 100 ml | | |

TLC: Cyclohexane, Acetone (3:1) with 0.25% Triethylamine

Procedure:

A-14.1 (1.2 g, 1.5 mmol) was dissolved in dichloromethane. Afterwards the solution was cooled with ice and 1.2 eq of pyridine was added, followed by 1.1 eq of propionic anhydride. Solution was stirred at room temperature overnight. Reaction was monitored via TLC and MS. Work up was done after two days, when sufficient product was detected. To extract product liquid-liquid extraction was performed. DCM solution was worked up with a saturated solution of ammonium chloride three times, followed by water three times. Organic phase was then dried with sodium sulphate and solvent evaporated to carry out white solid powder.

compound E-358 Yield 64% 97.5% purity

### Example 75. Synthesis of E-379-d

Reagents:

| | | | | |
|---|---|---|---|---|
| Reagent | Mol. Wt. | Properties | Amount | moles |

For the Reaction

| | | | | | | | |
|---|---|---|---|---|---|---|---|
| A-14.1 | 791 | g/mol | | 5 | g | 6.32 | mmol |
| Acetic Anhydride | 102.1 | g/mol | d = 1.1 g/mL | 20 | mL | 0.215 | mol |
| Nitric acid | 63.0 | g/mol | c = 65%; | 1.9 | mL | 27.2 | mmol |
| | | | d = 1.39 g/mL | | | | |

| | | |
|---|---|---|
| Glacial acetic | 40 | mL |
| acid | | |

| Thin Layer Chromatography | | Ratio |
|---|---|---|
| Chloroform | | 30 |
| Isopropanol | | 1 |
| Ammonia in | 7N solution | 1 |
| MeOH | | |
| Cyclohexane | | 10 |

Workup

| | |
|---|---|
| NaOH | 1M aqueous |
| Na₂CO₃ | |
| Na₂SO₄ (anhyd) | |

Procedure:
1. A-14.1 was taken up in glacial acetic acid in a 200-mL round bottom flask and magnetically stirred until dissolution (Note: initially clumps formed but eventually dissipated over time).
2. In another 100-mL round bottom flask, acetic anhydride was transferred and the reaction vessel cooled to 4° C. in an ice bath. Nitric acid was transferred to an addition funnel and slowly added dropwise to the cooled acetic anhydride.
3. When the A-14.1 is completely dissolved, the reaction vessel was also cooled to 4° C. in an ice bath.
4. To the cooled A-14.1 solution was added slowly the cooled acetic anhydride/nitric acid solution via an addition funnel keeping the reaction temperature <15° C.
5. Once the addition was complete, the ice bath was removed and the reaction mixture was continuously stirred and allowed to slowly warm to room temperature. (Note: some precipitation/cloudiness were observed but eventually disappeared at room temperature.)
6. In Process Control (IPC)—Reaction was progressively monitored via MS ([M+H] observed M+ without cladinose ring).
7. Reaction is complete upon disappearance of the A-14.1 peak in MS.

Workup:
8. Workup—The reaction solution was slowly poured onto 200 mL of ice/water mixture. To this was added under vigorous stirring 1 M aq. NaOH solution or with sodium carbonate (s) until basic pH.
9. The aqueous solution was extracted with DCM (3×75 mL). The combined organic phases were dried with anhydrous sodium sulfate, filtered and evaporated in vacuo.
10. Once completely evaporated, a solid powder (foam) was produced, which was dried under pressure.

Recrystallization/Purification:
11. The dried and weighed product was taken up in ethyl ether (10× the weight) and heated under reflux for 15-20 min.
12. The reaction mixture was gravity filtered while hot. The filtrate was cooled initially to room temperature and then stored in the refrigerator (4° C.) overnight.
13. The precipitated solids were filtered and the filtrate was evaporated in vacuo to afford the desired compound (3.7 g; 74% yield). Precipitate contains desired product and 2′-acetyled product. The acetyl group is easily removed through stirring in MeOH at room temperature
14. In Process Control (IPC)—Reaction was progressively monitored via MS ([M+H]).
15. Reaction is complete upon disappearance of the 2′-acetyled product peak in MS. 16. MeOH evaporated in vacuo.

Compound E-379-d Yield 74% m=3.7 g 97.0% purity

### Example 76. Synthesis of E-360-a

Chemicals:

| | | | |
|---|---|---|---|
| E-379-d | mw = 835.5 g/mol | m = 0.5 g | n = 0.60 mmol |
| Propionic anhydride | mw = 130.1 g/mol | V = 84.2 μL | n = 0.66 mmol |
| Pyridine | mw = 79.1 g/mol | V = 58.1 μL | n = 0.72 mmol |
| Dichloromethane | V = 30 ml | | |

TLC: Cyclohexane, Acetone (3:1) with 0.25% Triethylamine

Procedure:

E-379-d (0.5 g, 0.60 mmol) was dissolved in dichloromethane. Afterwards the solution was cooled with ice and 1.2 eq of pyridine was added, followed by 1.1 eq of propionic anhydride. Solution was stirred at room temperature overnight. Reaction was monitored via TLC and MS. Work up was done after three days, when sufficient product was detected. To extract product liquid-liquid extraction was performed. DCM solution was worked up with a saturated solution of ammonium chloride three times, followed by water three times. Organic phase was then dried with sodium sulphate and solvent evaporated to carry out white solid powder.

Compound E-360-a: Yield 76% 95.5% purity

### Embodiments

The invention described herein includes the following embodiments:
1. A preparation of comprising two compounds selected from: an Amphiphilic Lysosomally trapped Compound (ALC), and one of: compound A-1 to A-24, a zinc salt, and an anti-viral compound.
2. A preparation as in embodiment 1, wherein the Amphiphilic Lysosomally trapped Compound (ALC) is selected from Formulas 1, 2, 3 or 5.
3. A preparation as in embodiments 1-2, wherein the anti-viral compound is a serine protease inhibitor (e.g., camostat) or a cathepsin inhibitor.
4. A preparation as in embodiments 1-3, wherein the anti-viral compound is a serine protease inhibitor (e.g., camostat) or a cathepsin inhibitor.
5. A preparation as in embodiments 1-4, wherein the zinc salt is zinc orotate.
6. A preparation of comprising three compounds selected from: a compound selected from Formulas 1, 2, 3 or 5, a zinc salt, and an anti-viral compound.
7. A preparation of comprising an Amphiphilic Lysosomally trapped Compound (ALC), a compound selected from Formulas 1, 2, 3 or 5, and at least one of: glutathione, citrulline, arginine, a zinc salt, and an anti-viral compound.
8. A method of treating infection in a subject comprising administering to the subject two compounds selected from: an unconjugated Amphiphilic Lysosomally trapped Compound (ALC), a compound selected from Formulas 1, 2, 3 or 5, a zinc salt, and an anti-viral compound.
9. A method of treating infection in a subject comprising administering to the subject three compounds selected from: an unconjugated Amphiphilic Lysosomally trapped Compound (ALC), a compound selected from Formulas 1, 2, 3 or 5, a zinc salt, and an anti-viral compound.
10. A method of treating infection in a subject comprising administering to the subject an unconjugated Amphiphilic Lysosomally trapped Compound (ALC), a compound selected from Formulas 1, 2, 3 or 5, a zinc salt, and an anti-viral compound
11. A method of treating infection in a subject comprising administering to the subject a preparation of any one of embodiments 1-7.
12. A method of making a preparation of embodiments 1-7, comprising combining a first compound and a second compound, wherein each of the first compound and second compound is independently selected from: an unconjugated Amphiphilic Lysosomally trapped Compound (ALC), a compound selected from Formulas 1, 2, 3 or 5, a zinc salt, and an anti-viral compound.
13. A preparation of comprising two or more of: an unconjugated Amphiphilic Lysosomally trapped Compound (ALC), a compound selected from Formulas 1, 2 or 3, a zinc salt, and an anti-viral compound.
14. A preparation as in embodiment 13, wherein the unconjugated Amphiphilic Lysosomally trapped Compound (ALC) is selected from azithromycin or hydroxychloroquine.
15. A preparation as in embodiments 13-14, wherein the antiviral compound is a serine protease inhibitor (e.g., camostat) or a cathepsin inhibitor.
16. A preparation as in embodiments 13-15, wherein the antiviral compound is a serine protease inhibitor (e.g., camostat) or a cathepsin inhibitor.
17. A preparation as in embodiments 13-16, wherein the zinc salt is zinc orotate.
18. A preparation of comprising three or more of: an unconjugated Amphiphilic Lysosomally trapped Compound (ALC), a compound selected from Formulas 1, 2 or 3, a zinc salt, and an anti-viral compound.
19. A preparation as in embodiment 18, wherein the unconjugated Amphiphilic Lysosomally trapped Compound (ALC) is selected from azithromycin or hydroxychloroquine.
20. A preparation as in embodiments 18-19, wherein the antiviral compound is a serine protease inhibitor (e.g., camostat) or a cathepsin inhibitor.
21. A preparation as in embodiments 18-20, wherein the antiviral compound is a serine protease inhibitor (e.g., camostat) or a cathepsin inhibitor.
22. A preparation as in embodiments 18-21, wherein the zinc salt is zinc orotate.
23. A preparation of comprising an unconjugated Amphiphilic Lysosomally trapped Compound (ALC), a compound selected from Formulas 1, 2 or 3, a zinc salt, and an anti-viral compound.
24. A preparation as in embodiment 23, wherein the unconjugated Amphiphilic Lysosomally trapped Compound (ALC) is selected from azithromycin or hydroxychloroquine.
25. A preparation as in embodiments 23-24, wherein the antiviral compound is a serine protease inhibitor (e.g., camostat) or a cathepsin inhibitor.
26. A preparation as in embodiments 23-25, wherein the antiviral compound is a serine protease inhibitor (e.g., camostat) or a cathepsin inhibitor.
27. A preparation as in embodiments 23-26, wherein the zinc salt is zinc orotate.
28. A method of treating infection in a subject comprising administering to the subject two compounds selected from: an unconjugated Amphiphilic Lysosomally trapped Compound (ALC), a compound selected from Formulas 1, 2 or 3, a zinc salt, and an anti-viral compound.
29. A method of treating infection in a subject comprising administering to the subject three compounds selected from: an unconjugated Amphiphilic Lysosomally trapped Compound (ALC), a compound selected from Formulas 1, 2 or 3, a zinc salt, and an anti-viral compound.
30. A method of treating infection in a subject comprising administering to the subject an unconjugated Amphiphilic Lysosomally trapped Compound (ALC), a compound selected from Formulas 1, 2 or 3, a zinc salt, and an anti-viral compound
31. A method of treating infection in a subject comprising administering to the subject a preparation of any one of embodiments 13-27.
32. A method of making a preparation of embodiment 13, comprising combining a first compound and a second compound, wherein each of the first compound and second compound is independently selected from: an unconjugated Amphiphilic Lysosomally trapped Compound (ALC), a compound selected from Formulas 1, 2 or 3, a zinc salt, and an anti-viral compound.
33. A method of making a preparation of embodiment 13, comprising combining a first compound, a second compound, and a third compound, wherein each of the first compound, second compound, and third compound, is independently selected from: an unconjugated Amphiphilic Lysosomally trapped Compound (ALC), a compound selected from Formulas 1, 2 or 3, a zinc salt, and an anti-viral compound.
34. A method of making a preparation of embodiment 18, comprising providing a composition having a first compound and a second compound, and combining said preparation with a third compound, wherein each of the first compound, second compound, and third compound, is independently selected from: an unconjugated Amphiphilic Lysosomally trapped Compound (ALC), a compound selected from Formulas 1, 2 or 3, a zinc salt, and an anti-viral compound.

### OTHER EMBODIMENTS

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### ABBREVIATIONS

The following abbreviations were used as noted:
MeOH: methanol
NaHCO₃: sodium bicarbonate
K₂CO₃: potassium carbonate
MS: mass spectrometry
DMSO: dimethyl sulfoxide
TLC: thin layer chromatography
Et₃N: triethylamine
EtOAc: ethyl acetate
DCM: dichloromethane
NH₄Cl: ammonium chloride
THF: tetrahydrofuran
Na₂CO₃: sodium carbonate
EDCI: N-Ethyl-N′-(3-dimethylaminopropyl)carbodiimide hydrochloride
DMAP: 4-dimethylamino pyridine
HATU O-(7-Azabenzotriazol-1-yl)-N,N,N′,N′-tetramethyluronium-hexafluorphosphat
DIPEA N,N-Diisopropylethylamine

### CITATION LIST PATENT LITERATURE

US2007238882A1

US2003105066A1

US2008221158A1

US2008027012A1

U.S. Pat. No. 6,455,576B1

U.S. Pat. No. 5,677,287A

EP1748994B1

WO2007025632A2

WO9530641A1

WO0002567A1

US2012232257A1

### CITATIONS—NON PATENT LITERATURE

[1] L. J. Ignarro, Nitric Oxide Biology and Pathobiology, Academic Press, 2000, p. xvii.
[2] H. Al-Sa'doni, A. Ferro, 2000, Clinical Science, 98, pp 507-520.
[3] P. G. Wang et. al., Chem. Rev., 2002, 102 (4), pp 1091-1134.
[4] E. Oberdisse, E. Hackenthal, K. Kuschinsky, Pharmakologie und Toxikologie, Kapitel 16.2.6, Springer—Verlag, Berlin Heidelberg New York, 2001, pp 292-293.
[5] G. H. Hakimelahi, H. Sharghi, H. Zarrinmayeh, A. Khalafi-Nezhad, Helv. Chim. Acta. 1984, 67, 906-915, and literature cited therein.
[6] G. A. Olah et al., J. Org. Chem. 1990, 55 (17), 5179-5180.
[7] H. Burton, P. F. G. Praill, J. Chem. Soc. 1955, 729-731.
[8] E. Santaniello, M. Ravasi, P. Ferraboschi, J. Org. Chem. 1983, 48, 739-740.
[9] J. A. R. Rodrigues, A. P. O. Filho, P. J. S. Moran, Synth. Comm. 1999, 29 (12), 2169-2174.
[10] F. Francis et al., Berichte 1906, 39, 3798-3804.
[11] M. E. Kurz, E. P. Zahora, D. Layman, J. Org. Chem. 1973, 38 (13), 2277-2281.
[12] M. E. Kurz, E. Woodby, J. Org. Chem. 1976, 41 (14).
[13] Ronchetti D, Borghi V, Gaitan G, Herrero J F, Impagnatiello F. Br J Pharmacol. 2009 September; 158(2):569-79
[14] Lemaire S, Van Bambeke F, Tulkens P M. Antimicrob Agents Chemother. 2009 September; 53(9):3734-43
[15] Martinez L R, Han G, Chacko M, Mihu M R, Jacobson M, Gialanella P, Friedman A J, Nosanchuk J D, Friedman J M. J Invest Dermatol. 2009 October; 129(10):2463-9
[16] N. Pietrzik, C. Schips, T. Ziegler, Synthesis 2008, 519-526, and literature cited therein.
[17] R. Shan, C. Velaquez, E. E. Knaus, J. Med Chem. 2004, 47, 244-261.
[18] Volante, R. P., Tetrahed. Lett. 1981, 22, 3119-3122
[19] Silveira et al., Tetrahed. Lett. 2007, 48, 7469-7471

1. A preparation of comprising two compounds selected from: an Amphiphilic Lysosomally trapped Compound (ALC), and one of: compound A-1 to A-24, a zinc salt, and an anti-viral compound.

2. The preparation of claim 1, wherein the Amphiphilic Lysosomally trapped Compound (ALC) is selected from Formulas 1, 2, 3 or 5.

3. The preparation of claim 1, wherein the anti-viral compound is a serine protease inhibitor or a cathepsin inhibitor.

4. The preparation of claim 2, wherein the anti-viral compound is a serine protease inhibitor or a cathepsin inhibitor.

5. The preparation of claim 1, wherein the zinc salt is zinc orotate.

6. (canceled)

7. A preparation comprising an Amphiphilic Lysosomally trapped Compound (ALC), a compound selected from Formulas 1, 2, 3 or 5, and at least one of: glutathione, citrulline, arginine, a zinc salt, and an anti-viral compound.

8. A method of treating infection in a subject comprising administering to the subject two compounds selected from: an unconjugated Amphiphilic Lysosomally trapped Compound (ALC), a compound selected from Formulas 1, 2, 3 or 5, a zinc salt, and an anti-viral compound.

9. The method of claim 8, further comprising administering to the subject a third compound selected from: an unconjugated Amphiphilic Lysosomally trapped Compound (ALC), a compound selected from Formulas 1, 2, 3 or 5, a zinc salt, and an anti-viral compound.

10. (canceled)

11. A method of treating infection in a subject comprising administering to the subject a preparation of claim 1.

12. A method of making a preparation of claim 1, comprising combining a first compound and a second compound, wherein each of the first compound and second compound is independently selected from: an unconjugated Amphiphilic Lysosomally trapped Compound (ALC), a compound selected from Formulas 1, 2, 3 or 5, a zinc salt, and an anti-viral compound.
